(19) United States
(12) Patent Application Publication
(10) Pub. No.: US 2022/0169598 A1
(43) Pub. Date: Jun. 2, 2022

(54) HALOGENATED PHENYLSULFONAMIDE HYDROXAMIC ACID COMPOUNDS, COMPOSITIONS AND USES THEREOF AS SELECTIVE HDAC6 INHIBITORS

(71) Applicant: 2681603 ONTARIO INC., North York (CA)

(72) Inventors: Patrick T. Gunning, Mississauga (CA); Olasunkanmi Olaoye, Mississauga (CA); Krimo Toutah, Mississauga (CA); Andrew E. Shouksmith, Mississauga (CA); Justyna M. Shouksmith, Mississauga (CA); Nabanita Nawar, Toronto (CA); Yasir S. Raouf, Toronto (CA)

(21) Appl. No.: 17/593,877

(22) PCT Filed: Mar. 27, 2020
(86) PCT No.: PCT/IB2020/052965

§ 371 Date: Sep. 27, 2021

Related U.S. Application Data
(60) Provisional application No. 62/824,639, filed on Mar. 27, 2019.
(60) Provisional application No. 62/824,645, filed on Mar. 27, 2019.

(51) Int. Cl. C07C 311/19; C07C 259/10; C07D 213/53; C07D 237/08; C07D 239/26

(52) U.S. Cl. CPC C07C 311/19; C07C 259/10; C07D 239/26; C07D 237/08; C07D 213/53

(57) ABSTRACT

The present application relates to fluorinated benzylsulfonamide hydroxamic acid compounds of Formula I and/or pharmaceutically acceptable salt, solvate and/or prodrug thereof: (I) for use as a inhibitor of HDAC6. The application also relates to methods of treating a disease, disorder or condition using the compounds and compositions of the application.

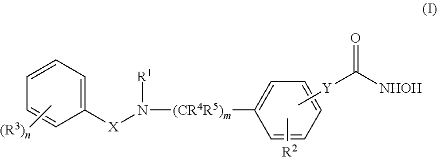

### RELATED APPLICATIONS

The present application claims the benefit of priority of U.S. provisional patent application No. 62/824,645 filed on Mar. 27, 2019, and U.S. provisional patent application No. 62/824,639 filed on Mar. 27, 2019, the contents of both of which are incorporated herein by reference in their entirety.

### FIELD

The present application relates to halogenated phenyl sulfonamide compounds and compositions and their use in selective HDAC6 inhibition in the treatment of various diseases and conditions.

### INTRODUCTION

Histone deacetylases (HDAC) is a family of structurally related aminohydrolases that target the terminal amino group on lysine residues. The majority of the HDAC family enzymes are known for their role in the control of gene transcription in the nucleus through modification of the tertiary structure of the DNA-histone complex. Unlike most of the HDACs, HDAC6 resides in the cytosol and targets non-histone substrates including cytoskeleton components α-tubulin, cortactin, and β-catenin, heat shock protein Hsp90, and redox regulatory protein peroxiredoxin. (Boyault et al., Oncology, 2007, 5468-5476) Therefore, HDAC6 modulates a wide range of cellular functions that are implicated in various stages of cancer. Because HDAC6 is structurally related yet functionally distinct from the other members of the HDAC family, selective inhibition of HDAC6 could be potentially useful in the treatment of various diseases including cancer.

HDAC6 inhibition has been shown to affect certain cancers (reviewed in Simms-Waldrip et al., Mol. Genet. Metabolism 2008, 94(3):283-286 and Rodriguez-Gonzalez et al., Cancer Res. 2008, 68(8):2557-2560), including: breast cancer (Lee et al., Cancer Res. 2008, 68(18):7561-7569); multiple myeloma (Hideshima et al., Proc. Natl. Acad. Sci. USA 2005, 102(24):8567-8572); pancreatic cancer (Nawrocki et al., Cancer Res. 2006, 66(7):3773-3781); lung cancer (Kamemura et al., Biochem. Biophys. Res. Commun. 2008, 374(1):84-89); prostate cancer (Mellado et al., Clin. Trans. Onco. 2009, 11(1):5-10); renal cancer (Cha et al., Clin. Cancer Res. 2009, 15(3):840-850); ovarian cancer (Bazzaro et al., Clin. Cancer Res. 2008, 14(22):7340-7347); and leukemias such as acute myeloid leukemia (AML) (Fiskus et al., Blood 2008, 112(7):2896-2905), and acute lymphoblastic leukemia (ALL) (Rodriguez-Gonzalez et al., Blood 2008, 112(11): Abstract 1923).

Other diseases shown to be influenced by inhibition of HDAC6 include cardiovascular diseases (Tannous et al., Circulation 2008, 117(24):3070-3078); bacterial infection (Dhakal and Mulve, J. Biol. Chem. 2008, 284(1):446-454); neurological diseases (reviewed in Kazantsev et al., Nat. Rev. Drug Disc. 2008, 7(10):854-868; see also Dompierre et al., J. Neurosci. 2007, 27(13):3571-3583; Kozikowski et al., J. Med. Chem. 2007, 50:3054-3061); and inflammation and immunological disorders such as rheumatoid arthritis, psoriasis, multiple sclerosis, lupus and organ transplant rejection (reviewed in Wang et al., Nat. Rev. Drug Disc. 2009, 8(12):969-981).

HDAC6 inhibitors can also be used in combination with other active agents. Some examples include: chemotherapeutics, microtubule destabilizing agents, Hsp90 inhibitors, inhibitors of Hsp90 downstream proteins, tyrosine kinase inhibitors, HER-2 inhibitors, BCR-ABL inhibitors, Akt inhibitors, c-Raf and MEK inhibitors, Aurora A and B inhibitors, EGFR inhibitors, proteasome inhibitors, ubiquitin proteasome system inhibitors, modulators of autophagy and protein homeostasis agents.

WO 2006/017214 A2 discloses para-sulfonamide benzylic hydroxamic acid compounds for use as HDAC inhibitors for treating neurodegenerative diseases and cancer.

U.S. Pat. No. 9,382,197 B2 discloses HDAC6 selective phenylsulfonamide benzylic hydroxamic acid compounds for the treatment of cancer, inflammatory, and neurological.

US 2003/0013757 A1 discloses aromatic dicarboxylic acid derivatives and the method of preparation thereof for the treatment of cancer.

### SUMMARY

The present application describes a novel class of compounds showing selective inhibition of HDAC6 and having strong anti-cancer activity. Strong cancer-killing potency (e.g. IC₅₀<5 μM) of exemplary compounds has been demonstrated in various cell cultures, such as major types of acute myeloid leukemia (AML) and adenocarcinoma, including in patient-derived cells. In addition to strong anti-cancer activity, exemplary compounds of the application were found to meet and/or exceed other clinically desired parameters, including high metabolic stability.

Accordingly, in some embodiments, the present application includes a compound of Formula I or a pharmaceutically acceptable salt, solvate, and/or prodrug thereof:

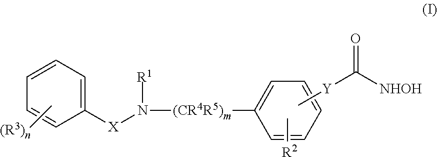

wherein
n is 4 or 5;
m is 0, 1, 2, 3, or 4;
X is selected from C(O) and SO₂;
R¹ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R² is selected from H, halo, C₁₋₄alkyl, and OC₁₋₄alkyl;
each R³ is the same or different and is selected from halo;
R⁴ and R⁵ are independently selected from H and C₁₋₄alkyl;
Y is absent or selected from C₁₋₆alkyene, C₂₋₆alkenylene and C₂₋₆alkynylene;
the —Y—C(O)NHOH group is bonded to a meta or para position of the phenyl ring;
all alkyl and alkylene are optionally fluoro substituted; and
all available hydrogen atoms are optionally replaced with deuterium,
provided when m is 0, and Y is absent, then R¹ is not H or C₁₋₁₀alkyl.

The present application also includes a compound of Formula I wherein the compound of Formula I is a compound of Formula I-A or a pharmaceutically acceptable salt, solvate, and/or prodrug thereof:

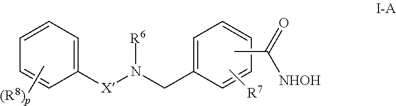

wherein
p is 4 or 5;
X′ is selected from C(O) and SO₂;
R⁶ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 7 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R⁷ is selected from H, halo, C₁₋₄alkyl, and OC₁₋₄alkyl;
each R⁸ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

The present application also includes a compound of Formula I, wherein the compound of Formula I is a compound of Formula I-B, or a pharmaceutically acceptable salt and/or solvate thereof:

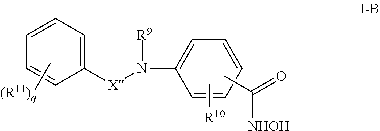

wherein
q is 4 or 5;
X″ is selected from C(O) and SO₂;
R⁹ is selected from C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, each of which is optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R¹⁰ is selected from H, halo, C₁₋₄alkyl, and OC₁₋₄alkyl;
each R¹¹ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

In some embodiments, the present application includes a compound of Formula I wherein the compound of Formula I is a compound of Formula I-C or a pharmaceutically acceptable salt, solvate, and/or prodrug thereof:

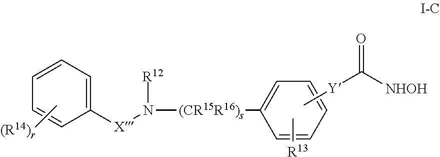

wherein
r is 4 or 5;
s is 0, 1, 2, 3, or 4;
X′″ is selected from C(O) and SO₂;
R¹² is selected from H, C₁₋₁₀alkyl, C₃₋₁₀cycloalkyl, C₁₋₆alkyleneC₃₋₁₀cycloalkyl, C₁₋₆ alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R¹³ is selected from H, halo, C₁₋₄alkyl, and OC₁₋₄alkyl;
each R¹⁴ is the same or different and is selected from halo;
R¹⁵ and R¹⁶ are independently selected from H and C₁₋₄alkyl;
Y′ is selected from C₁₋₆alkyene, C₂₋₆alkenylene and C₂₋₆alkynylene;
the —Y′—C(O)NHOH group is bonded to a meta or para position of the phenyl ring;
all alkyl and alkylene are optionally fluoro substituted; and
all available hydrogen atoms are optionally replaced with deuterium.

In some embodiments, the present application includes a composition comprising one or more compounds of Formula I, I-A, I-B and/or I-C, and/or salts, solvates and/or prodrugs thereof, and one or more carriers. In some embodiments, the composition is a pharmaceutical composition and the one or more carriers are pharmaceutically acceptable.

In some embodiments, the present application includes a use of one or more compounds or compositions of the application as a medicament.

In some embodiments, the present application includes a method of selectively inhibiting histone deacetylase 6 (HDAC6) in a cell comprising administering an effective amount of one or more compounds or compositions of the application to the cell.

In some embodiments, the present application includes a method of treating a disease, disorder or condition that benefits from inhibiting HDAC6 comprising administering an effective amount of one or more compounds or compositions of the application to a subject in need thereof.

Other features and advantages of the present application will become apparent from the following detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The embodiments of the application will now be described in greater detail with reference to the attached drawings in which:

FIG. 1 shows the Western blot analysis of MV-4-11 cells treated with exemplary compound I-4. Panel A shows the band for Ac-α-tubulin; Panel B shows the band for Ac-Histone 3.

FIG. 2 shows the Western blot analysis of MV-4-11 cells treated with exemplary compound I-18, showing the bands for Ac-α-tubulin and Ac-Histone 3.

FIG. 3 shows the Western blot analysis of MV-4-11 cells treated with exemplary compound I-3, showing the bands for Ac-α-tubulin and Ac-Histone 3.

FIG. 4 shows the Western blot analysis of MV-4-11 cells treated with Ricolinostat, showing the bands for Ac-α-tubulin, Ac-Histone 3 and unacetylated Histone 3.

FIG. 5 shows the Western blot analysis of MV-4-11 cells treated with exemplary compound I-13, showing the bands for Ac-α-tubulin, Ac-Histone 3 and unacetylated Histone 3.

FIG. 6 shows the Western blot analysis of MV-4-11 cells treated with exemplary compound I-12, showing the bands for Ac-α-tubulin, Ac-Histone 3 and unacetylated Histone 3.

FIG. 7 shows the FACS analysis of cells treated with exemplary compound I-13 at increasing concentrations with DMSO as control.

FIG. 8 shows the FACS analysis of cells treated with exemplary compound I-18 at increasing concentrations with DMSO as control.

FIG. 9 shows the plasma concentration in mice of exemplary compound I-13.

FIG. 10 shows the plasma concentration in mice of exemplary compound I-50.

FIG. 11 shows the Western blot analysis of MV-4-11 cells treated with exemplary compound I-50 and citarinostat showing the bands for Ac-α-tubulin and Ac-Histone 3.

FIG. 12 shows the Western blot analysis of MM.1 S cells treated with exemplary compound I-50 and citarinostat showing the bands for Ac-α-tubulin and Ac-Histone 3.

FIG. 13 shows the Western blot analysis of MV-4-11 cells treated with exemplary compound I-34 and citarinostat showing the bands for Ac-α-tubulin and Ac-Histone 3.

FIG. 14 shows the Western blot analysis of MV-4-11 cells treated with exemplary compound I-13 and citarinostat showing the bands for Ac-α-tubulin and Ac-Histone 3.

FIG. 15 shows the FACS analysis of cells treated with exemplary compound I-50 at increasing concentrations with DMSO as control.

FIG. 16 shows the FACS analysis of cells treated with exemplary compound I-34 at increasing concentrations with DMSO as control.

FIG. 17 shows the FACS analysis of cells treated with exemplary compound I-13 at increasing concentrations with DMSO as control.

FIG. 18 shows the immunofluorescence analysis of HeLa cells staining for a-tubulin acetylation and histone H3 acetylation following 6h treatment with exemplary compound I-13 and citarinostat.

FIG. 19 shows the Immunofluorescence analysis of HeLa cells staining for a-tubulin acetylation and histone H3 acetylation following 6 h treatment with exemplary compound I-34.

FIG. 20 shows the displacement of fluorescent probe from HDAC6 enzyme by increasing concentrations of exemplary compound I-50 and citarinostat.

FIG. 21 shows the plasma concentration in mice of exemplary compound I-25.

FIG. 22 shows the plasma concentration in mice of exemplary compound I-19.

FIG. 23 shows the plasma concentration in mice of exemplary compound I-18.

FIG. 24 shows the plasma concentration in mice of exemplary compound I-63.

### DESCRIPTION OF VARIOUS EMBODIMENTS

### 1. Definitions

Unless otherwise indicated, the definitions and embodiments described in this and other sections are intended to be applicable to all embodiments and aspects of the present application herein described for which they are suitable as would be understood by a person skilled in the art.

The term “compound of the application” or “compound of the present application” and the like as used herein refers to a compound of Formula I, I-A, I-B and/or I-C, including pharmaceutically acceptable salts, solvates and/or prodrugs thereof.

The term “composition of the application” or “composition of the present application” and the like as used herein refers to a composition, such a pharmaceutical composition, comprising one or more compounds of the application.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a compound” should be understood to present certain aspects with one compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “agent” as used herein indicates a compound or mixture of compounds that, when added to a composition, tend to produce a particular effect on the composition's properties.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown or named in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms that the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The present application refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “haloalkyl” as used herein refers to an alkyl group wherein one or more, including all of the hydrogen atoms are replaced by a halogen atom.

The term “halosubstituted” as used herein refers to a chemical group wherein one or more, including all of the hydrogen atoms, are replaced by a halogen atom.

The term “fluoroosubstituted” as used herein refers to a chemical group wherein one or more, including all of the hydrogen atoms, are replaced by a fluorine atom.

The term “optionally substituted” refers to groups, structures, or molecules that are either unsubstituted or are substituted with one or more substituents.

The term “cycloalkyl,” as used herein, whether it is used alone or as part of another group, means a saturated carbocyclic group containing a number of carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. When a cycloalkyl group contains more than one ring, the rings may be fused, bridged, spirofused or linked by a bond.

The term “aryl” as used herein, whether it is used alone or as part of another group, refers to cyclic groups containing from 6 to 10 carbon atoms and one or more rings, at least one of which is aromatic ring. When an aryl group contains more than one ring, the rings may be fused, bridged, spirofused or linked by a bond. In some embodiments of the application, the aryl group contains from 6, 9 or 10 carbon atoms, such as phenyl, indanyl or naphthyl.

The term “heterocycloalkyl” as used herein, whether it is used alone or as part of another group, refers to cyclic groups containing 3 to 10 atoms, and at least one non-aromatic ring in which one or more of the atoms are a heteroatom selected from O, S, and N. Heterocycloalkyl groups are either saturated or unsaturated (i.e. contain one or more double bonds) and contain one or more than one ring (i.e. are polycyclic). When a heterocycloalkyl group contains more than one ring, the rings may be fused, bridged, spirofused or linked by a bond. When a heterocycloalkyl group contains the prefix Cₙ₁₋ₙ₂ this prefix indicates the number of carbon atoms in the corresponding carbocyclic group in which one or more of the ring atoms is replaced with a heteroatom as defined above. Examples of heterocycloalkyl groups include aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran, tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine, homopiperazine, 1,3-dioxepane, 4,7-dihydro-1,3-dioxepin, and hexamethylene oxide.

The term “heteroaryl” as used herein refers to cyclic groups containing 5, 6, 9 or 10 atoms, one or more rings, at least one of which is aromatic ring, and at least one heteroatom selected from O, S, and N. When a heteroaryl group contains more than one ring, the rings may be fused, bridged, spirofused or linked by a bond. When a heteroaryl group contains the prefix Cₙ₁₋ₙ₂ this prefix indicates the number of carbon atoms in the corresponding carbocyclic group in which one or more of the ring atoms is replaced with a heteroatom as defined above. Examples of heteroaryl groups include monocyclic aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole. Additionally, heteroaryl encompasses polycyclic aromatic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

A first ring group being “fused” with a second ring group means the first ring and the second ring share at least two atoms there between.

A first ring being “bridged” with a second ring means the first ring and the second ring share two non-adjacent atoms there between.

A first ring being “spirofused” with a second ring means the first ring and the second ring share one atom there between.

The term “available”, as in “available hydrogen atoms” or “available atoms” refers to atoms that would be known to a person skilled in the art to be capable of replacement by a substituent.

The terms “halo” or “halogen” as used herein, whether it is used alone or as part of another group, refers to a halogen atom and includes fluoro, chloro, bromo and iodo.

The term “atm” as used herein refers to atmosphere.

The term “MS” as used herein refers to mass spectrometry.

The term “aq.” As used herein refers to aqueous.

The term DCM as used herein refers to dichloromethane.

The term DIPEA as used herein refers to N,N-diisopropyl ethylamine

The term DMF as used herein refers to dimethylformamide.

The term THF as used herein refers to tetrahydrofuran.

The term DMSO as used herein refers to dimethylsulfoxide.

The term EtOAc as used herein refers to ethyl acetate.

The term MeOH as used herein refers to methanol.

The term MeCN as used herein refers to acetonitrile.

The term HCl as used herein refers to hydrochloric acid.

The term TFA as used herein refers to trifluoroacetic acid.

The term CV as used herein refers to column volume.

The term Hex as used herein refers to hexanes.

The term PBS as used herein refers to phosphate-based buffer.

The term Epi as used herein refers to Eppendorf tubes.

The term MW as used herein refers to molecular weight.

The term HPLC as used herein refers to high performance liquid chromatography.

The term LCMS as used herein refers to liquid chromatography-mass spectrometry.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications

The term “pharmaceutically acceptable” means compatible with the treatment of a subject.

The term “pharmaceutically acceptable carrier” means a non-toxic solvent, dispersant, excipient, adjuvant and/or other material, which is mixed with the active ingredient in order to permit the formation of a pharmaceutical composition, i.e., a dosage form capable of administration to a subject.

The term “pharmaceutically acceptable salt” means either an acid addition salt or a base addition salt, which is suitable for, or compatible with, the treatment of a subject.

An acid addition salt suitable for, or compatible with, the treatment of subjects is any non-toxic organic or inorganic acid addition salt of any basic compound of the application.

A base addition salt suitable for, or compatible with, the treatment of subjects is any non-toxic organic or inorganic base addition salt of any acidic compound of the application.

The term “solvate” as used herein means a compound, or a salt of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

As used herein, a “disease, disorder or condition that benefits from selectively inhibiting HDAC6” as used herein refers to a disease, disorder or condition treatable by inhibition of HDAC6 activity and particularly using an HDAC6 inhibitor, such as a compound of the application herein described.

The term “benefits from selectively inhibiting HDAC6” as used herein means that the disease, disorder or condition to be treated is affected by, modulated by and/or has some biological basis, either direct or indirect, that includes aberrant HDAC6 activity, in particular, increased HDAC6 activity. These diseases respond favourably when HDAC6 activity associated with the disease, disorder or condition is inhibited by one or more of the compounds or compositions of the application.

The term “inhibit” or “inhibition” and the like as used herein means any reduction or decrease in activity, detectable directly or indirectly, in the presence of a compound compared to activity under otherwise identical conditions, except in the absence of the compound.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

As used herein, the term “effective amount” or “therapeutically effective amount” means an amount of one or more compounds or compositions of the application that is effective, at dosages and for periods of time necessary to achieve the desired result. In an embodiment, effective amounts vary according to factors such as the disease state, age, sex and/or weight of the subject. In a further embodiment, the amount of a given compound or composition that will correspond to an effective amount will vary depending upon factors, such as the given compound(s), the pharmaceutical formulation, the route of administration, the type of condition, disease or disorder, the identity of the subject being treated, and the like, but can nevertheless be routinely determined by one skilled in the art.

The term “administered” as used herein means administration of a therapeutically effective amount of one or more compounds or compositions of the application to a cell, tissue, organ or subject.

The term “selectively inhibiting Histone deacetylase 6” or “selectively inhibiting HDAC6” as used herein means HDAC6 is inhibited to a greater degree than the other members of the HDAC family.

The term “IC₅₀” as used herein refers to the half-maximal inhibitory concentration of a compound and is the concentration of an inhibitor where the response (or binding) is reduced by half.

The term “cell proliferative disorder” as used herein refers to a disease, disorder or condition characterized by cells that have the capacity for autonomous growth or replication, e.g., an abnormal state or condition characterized by proliferative cell growth.

The term “neoplasm” as used herein refers to a mass of tissue resulting from the abnormal growth and/or division of cells in a subject having a cell proliferative disorder. Neoplasms can be benign (such as uterine fibroids and melanocytic nevi), potentially malignant (such as carcinoma in situ) or malignant (i.e. cancer).

### II. Compounds and Compositions of the Application

In one aspect, the present application includes a compound of Formula I or a pharmaceutically acceptable salt, solvate, and/or prodrug thereof:

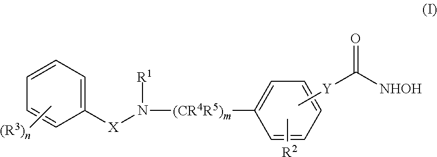

wherein
n is 4 or 5;
m is 0, 1, 2, 3, or 4;
X is selected from C(O) and SO₂;
R¹ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆ heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R² is selected from H, halo, C₁₋₄alkyl, and OC₁₋₄alkyl;
each R³ is the same or different and is selected from halo;
R⁴ and R⁵ are independently selected from H and C₁₋₄alkyl;
Y is absent or selected from C₁₋₆alkyene, C₂₋₆alkenylene and C₂₋₆alkynylene;
the —Y—C(O)NHOH group is bonded to a meta or para position of the phenyl ring;
all alkyl and alkylene are optionally fluoro substituted; and
all available hydrogen atoms are optionally replaced with deuterium,
provided when m is 0, and Y is absent, then R¹ is not H or C₁₋₁₀alkyl.

In some embodiments, R¹ is selected from H, C₁₋₅alkyl, C₁₋₃alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆ heterocycloalkyl, phenyl, and C₅₋₆heteroayl are each unsubstituted or substituted with one or more C₁₋₄alkyl or halo.

In some embodiments, R¹ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, C₁₋₃alkyl, N(C₁₋₄alkyl)(C₁₋₄alkyl), and OC₁₋₄alkyl. In some embodiments, R¹ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, C₁₋₄alkyl, N(C₁₋₄alkyl)(C₁₋₄alkyl), and OC₁₋₄alkyl. In some embodiments, R¹ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups are optionally substituted with one or more groups selected from fluorine, C₁₋₄alkyl, N(C₁₋₂alkyl)(C₁₋₂alkyl), and OC₁₋₂alkyl. In some embodiments, R¹ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups optionally substituted with one or more groups independently selected from F, C₁₋₄alkyl, N(CH₃)₂, and OCH₃.

In some embodiments, R¹ is benzyl optionally substituted with one or more groups selected from fluorine, C₁₋₄alkyl, N(C₁₋₂alkyl)(C₁₋₂alkyl), and OC₁₋₂alkyl. In some embodiments, R¹ is pyridinylmethyl, pyridazinylmethyl, or pyrimidinylemethyl optionally substituted with one or more groups selected from fluorine, C₁₋₄alkyl, N(C₁₋₂alkyl)(C₁₋₂alkyl), and OC₁₋₂alkyl. In some embodiments, R¹ is pyridinylmethyl, pyridazinylmethyl, or pyrimidinylemethyl. In some embodiments, R¹ is pyridinylmethyl optionally substituted with one or more groups selected from fluorine, C₁₋₄alkyl, N(C₁₋₂alkyl)(C₁₋₂alkyl), and OC₁₋₂alkyl. In some embodiments, R¹ is methoxyethyl. In some embodiments, R¹ is C₃₋₆cycloalkyl. In some embodiments, R¹ is isopropyl or cyclopentyl.

In some embodiments, R¹ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, and C₁₋₃alkyl. In some embodiments, R¹ is selected from H, methyl, isopropyl, C₃₋₆cycloalkyl, benzyl, and pyridinylmethyl, the latter 2 groups are optionally substituted with one or more groups selected from fluorine and C₁₋₂alkyl. In some embodiments, R¹ is pyridinylmethyl. In some embodiments, R¹ is C₃₋₆cycloalkyl. In some embodiments, R¹ is isopropyl or cyclopentyl.

In some embodiments, in compounds of Formula I, R² is selected from H, halo and OC₁₋₃alkyl. In some embodiments, in compounds of Formula I, R² is selected from H, halo and OCH₃. In some embodiments, R² is selected from H, fluorine and OCH₃.

In some embodiments, in compounds of Formula I, R² is selected from H, and halo.

In some embodiments, R² is selected from H, and fluorine.

In some embodiments, X is SO₂. In other embodiments, X is C(O).

In some embodiments, each R³ is selected from F and Cl. In some embodiments, each R³ is F. In some embodiments, one R³ is Cl and the remaining R³ are F. In some embodiments, two R³ are Cl and the remaining R³ are F.

In some embodiments, R⁴ and R⁵ are independently selected from H and CH₃. In some embodiments, both R⁴ and R⁵ are H.

In some embodiments, m is 0, 1 or 2. In some embodiments, m is 0 or 1. In some embodiments, m is 1.

In some embodiments, Y is absent.

In some embodiments, Y is selected from C₁₋₄alkyene, C₂₋₄alkenylene and C₂₋₄alkynylene. In some embodiments, Y is selected from C₁₋₄alkyene and C₂₋₄alkenylene. In some embodiments, Y is selected from —CH₂—, —CH₂CH₂— and —CH═CH—. In some embodiments, Y is —CH═CH—. In some embodiments, Y is —CH═CH— and the double bond is in the trans configuration.

In some embodiments, the —Y—C(O)NHOH group is bonded to the para position of the phenyl ring.

In one aspect, the present application includes a compound of Formula I wherein the compound of Formula I is a compound of Formula I-A or a pharmaceutically acceptable salt, solvate, and/or prodrug thereof:

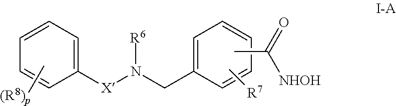

wherein
p is 4 or 5;
X′ is selected from C(O) and SO₂;
R⁶ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 7 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R⁷ is selected from H, halo, C₁₋₄alkyl, and OC₁₋₄alkyl;
each R⁸ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

In some embodiments, R⁶ is selected from H, C₁₋₅alkyl, C₁₋₃alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo.

In some embodiments, R⁶ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, C₁₋₃alkyl, N(C₁₋₄alkyl)(C₁₋₄alkyl), and OC₁₋₄alkyl. In some embodiments, R⁶ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, C₁₋₄alkyl, N(C₁₋₄alkyl)(C₁₋₄alkyl), and OC₁₋₄alkyl. In some embodiments, R⁶ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups are optionally substituted with one or more groups selected from fluorine, C₁₋₄alkyl, N(C₁₋₂alkyl)(C₁₋₂alkyl), and OC₁₋₂alkyl. In some embodiments, R⁶ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups optionally substituted with one or more groups independently selected from F, C₁₋₄alkyl, N(CH₃)₂, and OCH₃.

In some embodiments, R⁶ is benzyl optionally substituted with one or more groups selected from fluorine, C₁₋₄alkyl, N(C₁₋₂alkyl)(C₁₋₂alkyl), and OC₁₋₂alkyl. In some embodiments, R⁶ is pyridinylmethyl, pyridazinylmethyl, or pyrimidinylemethyl optionally substituted with one or more groups selected from fluorine, C₁₋₄alkyl, N(C₁₋₂alkyl)(C₁₋₂alkyl), and OC₁₋₂alkyl. In some embodiments, R⁶ is pyridinylmethyl, pyridazinylmethyl, or pyrimidinylemethyl. In some embodiments, R⁶ is pyridinylmethyl optionally substituted with one or more groups selected from fluorine, C₁₋₄alkyl, N(C₁₋₂alkyl)(C₁₋₂alkyl), and OC₁₋₂alkyl. In some embodiments, R⁶ is methoxyethyl. In some embodiments, R⁶ is C₃₋₆cycloalkyl. In some embodiments, R⁶ is isopropyl or cyclopentyl.

In some embodiments, R⁶ is selected from H, C₁₋₃alkyl, C₃cycloalkyl, C₁₋₂alkyleneheteroaryl, and C₁₋₂alkylenearyl, the latter 4 groups optionally substituted with one or more groups independently selected from halo, and C₁₋₃alkyl.

In some embodiments, R⁶ is selected from H, methyl, isopropyl, cyclopropyl, benzyl, and pyridinylmethyl, the latter 2 groups are optionally substituted with one or more groups selected from fluorine and C₁₋₂alkyl. In some embodiments, R⁶ is pyridinylmethyl. In some embodiments, R⁶ is isopropyl.

In some embodiments, R⁷ is selected from H, halo and OC₁₋₃alkyl. In some embodiments, R⁷ is selected from H, halo and OCH₃. In some embodiments, R⁷ is selected from H, fluorine and OCH₃.

In some embodiments, in compounds of Formula I-A, R⁷ is selected from H, and halo.

In some embodiments, R⁷ is selected from H, and fluorine.

In some embodiments, X′ is SO₂. In other embodiments, X′ is C(O).

In some embodiments, each R⁸ is selected from F and Cl. In some embodiments, each R⁸ is F. In some embodiments, one R⁸ is Cl and the remaining R⁸ are F. In some embodiments, two R⁸ are Cl and the remaining R⁸ are F.

In some embodiments, the C(O)NHOH group in the compound of Formula I-A is bonded to the para position of the phenyl ring.

In some embodiments, the compound of Formula I of the present application is a compound of Formula I-A selected from:

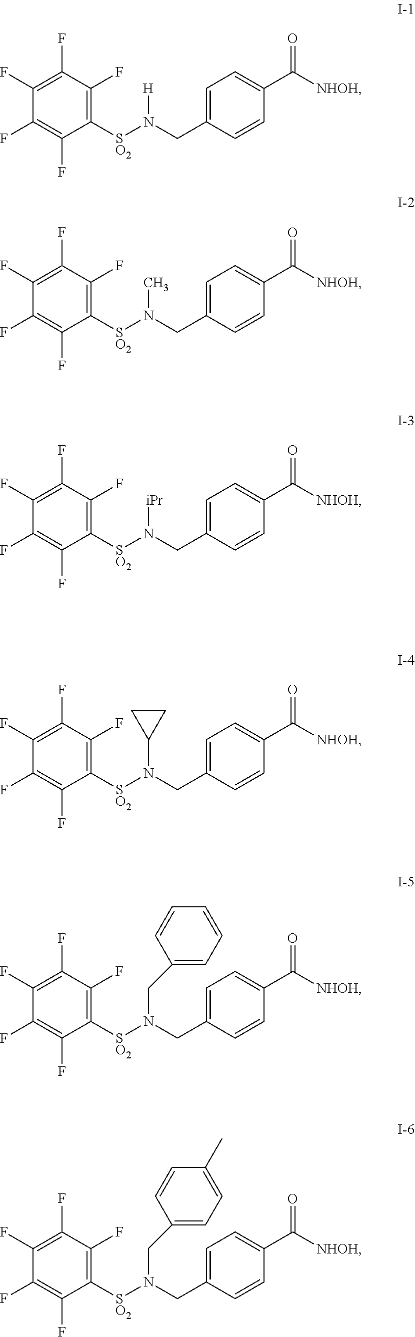

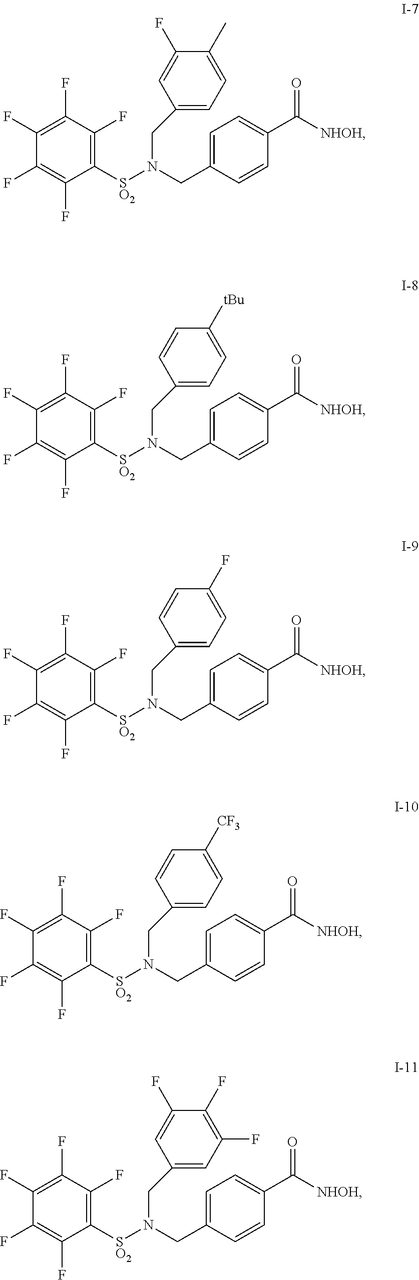

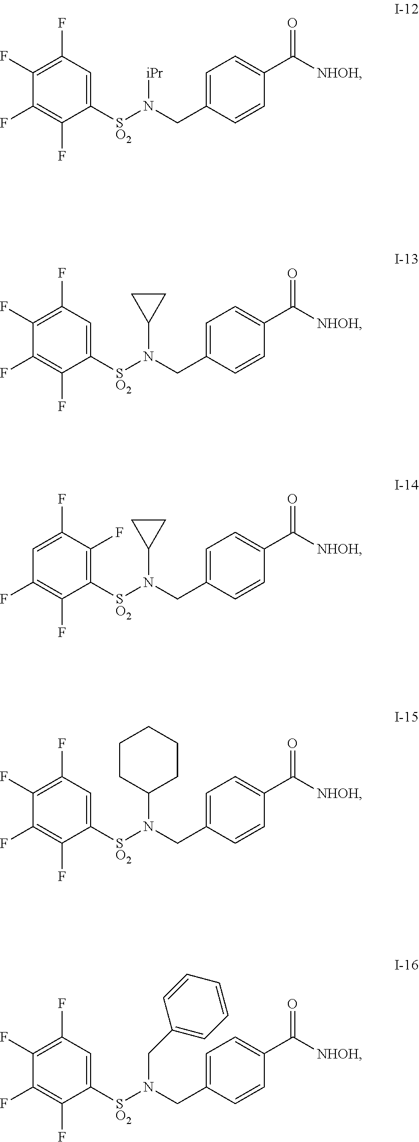

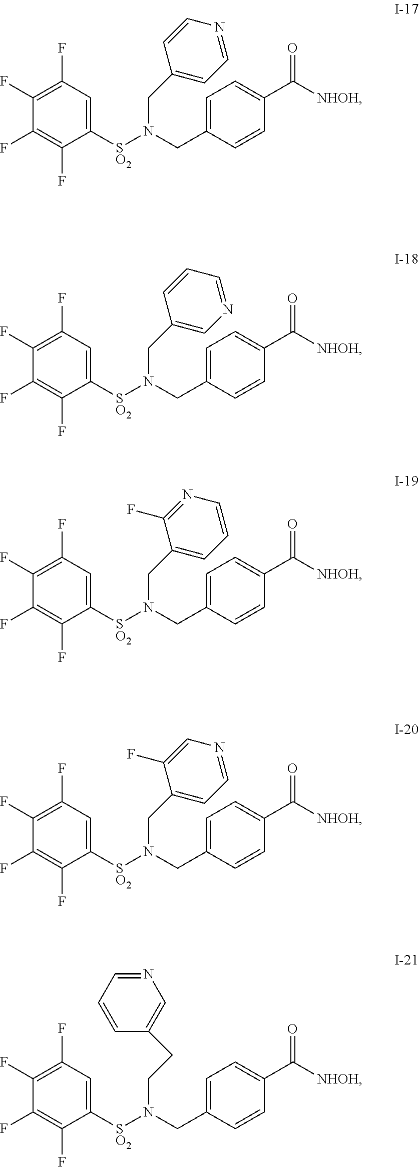

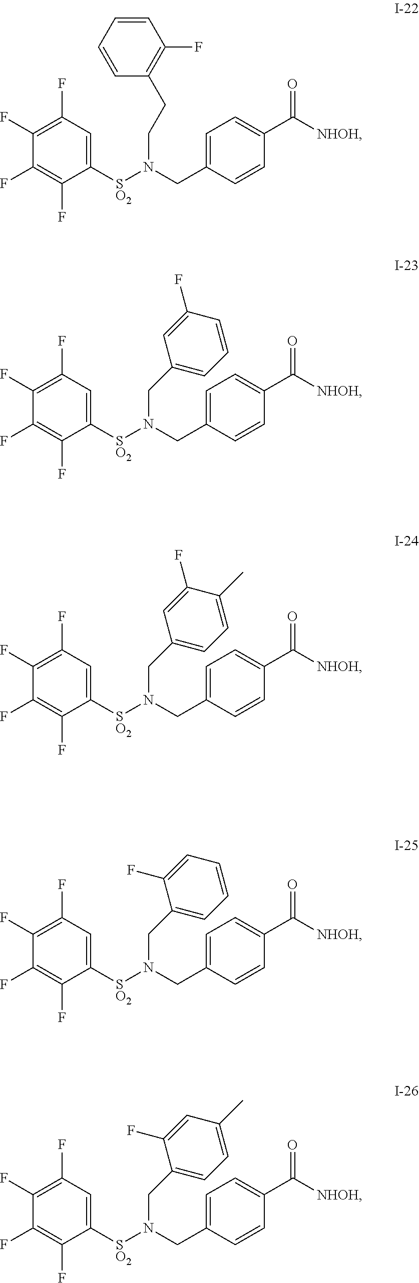

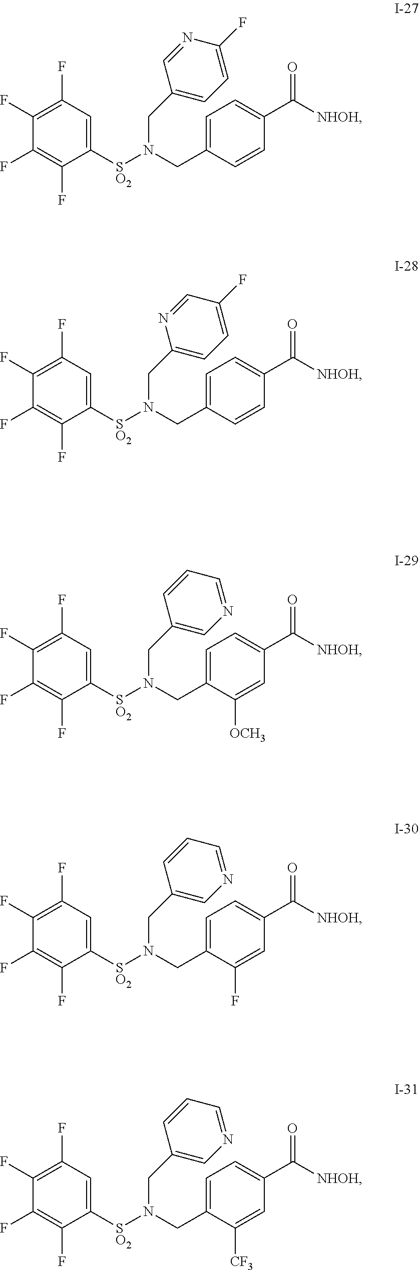

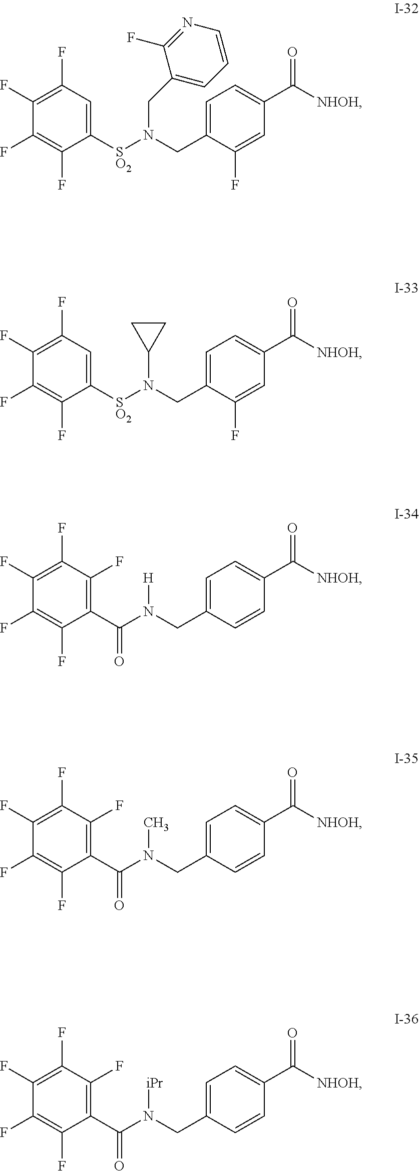

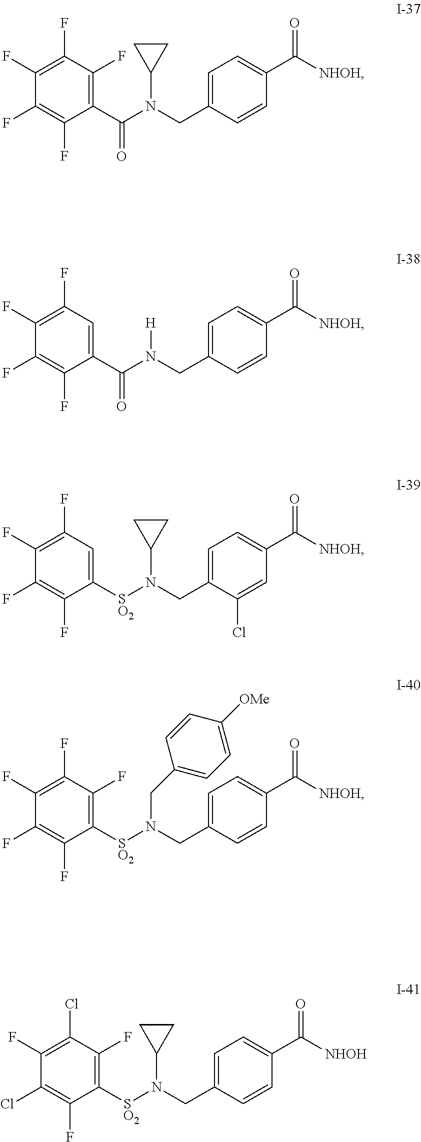

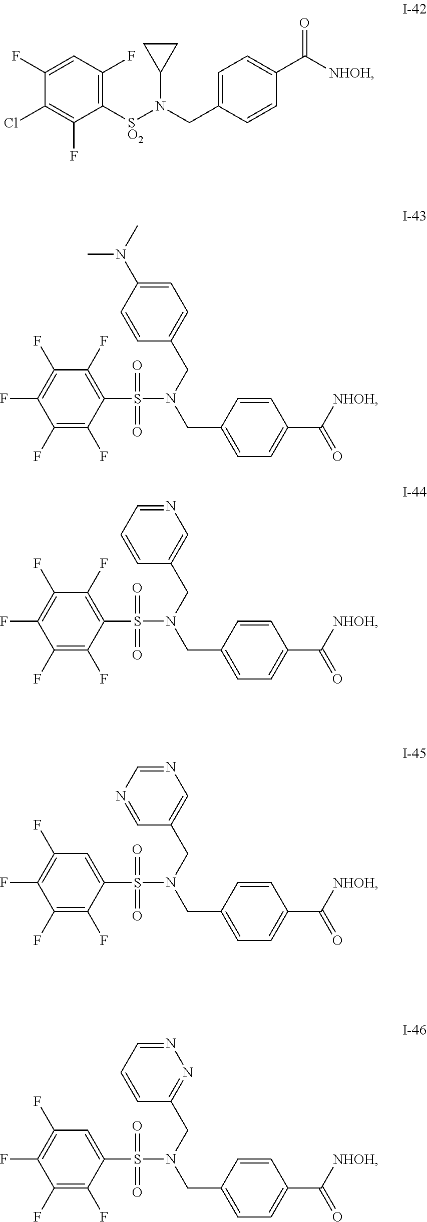

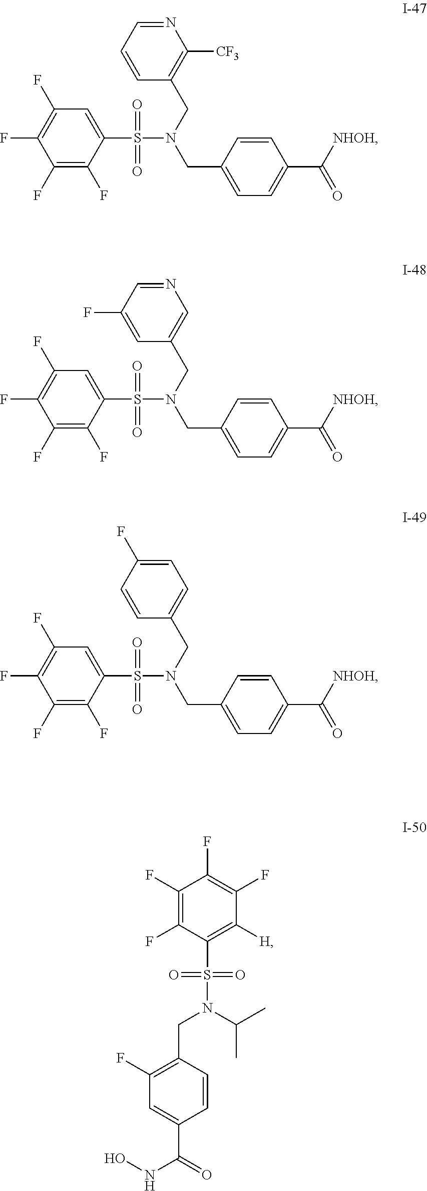

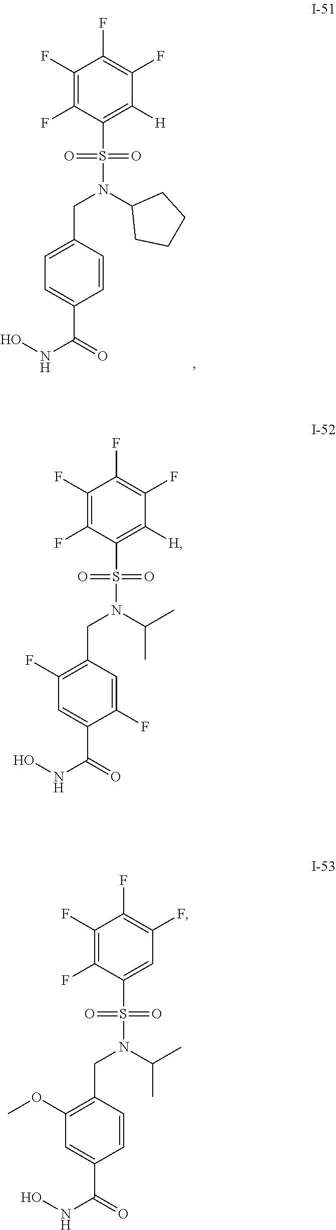

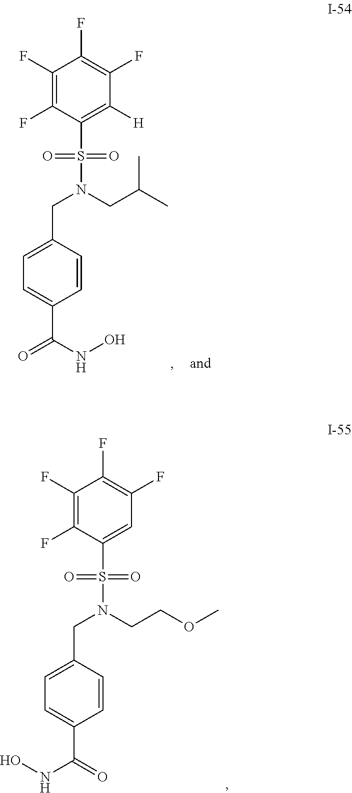 [SMILES: CC(C)CN(Cc1ccc(C(=O)NO)cc1)S(=O)(=O)c1cc(F)c(F)c(F)c1F ; COCCN(Cc1ccc(C(=O)NO)cc1)S(=O)(=O)c1cc(F)c(F)c(F)c1F]

or a pharmaceutically acceptable salt, solvate and/or prodrug thereof.

In some embodiments, the compound of Formula I is a compound of Formula I-A selected from

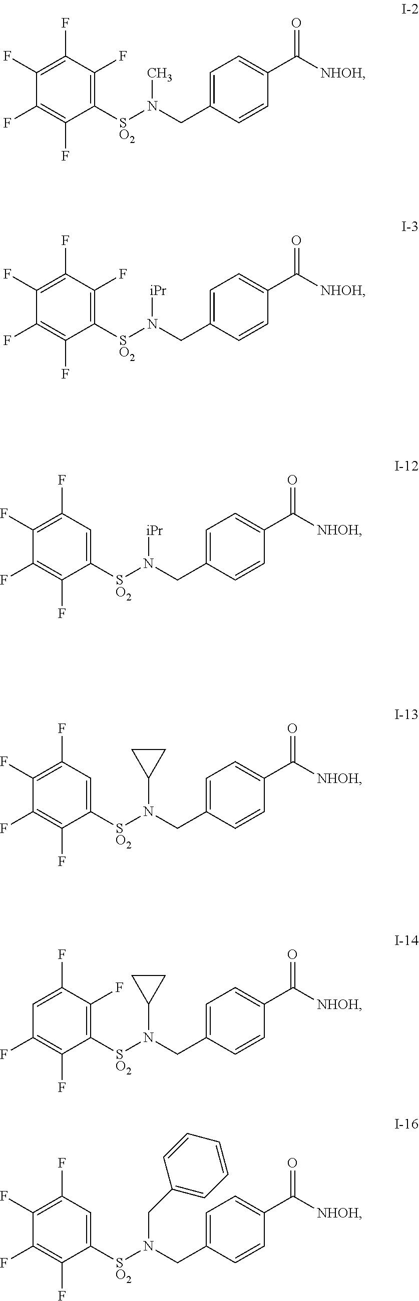

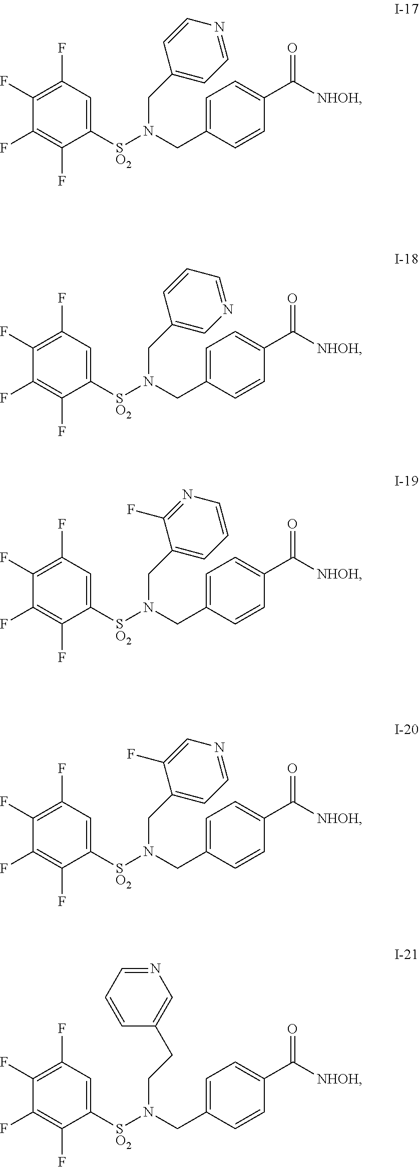

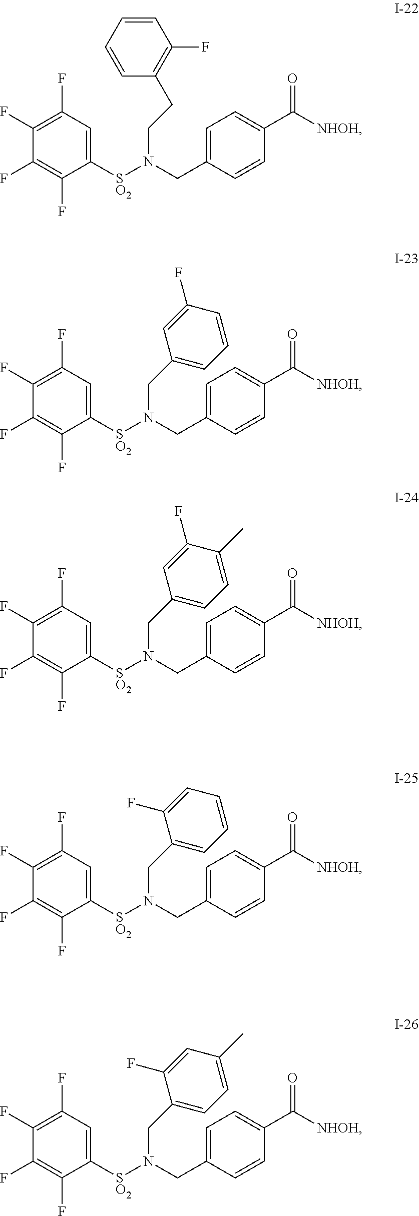

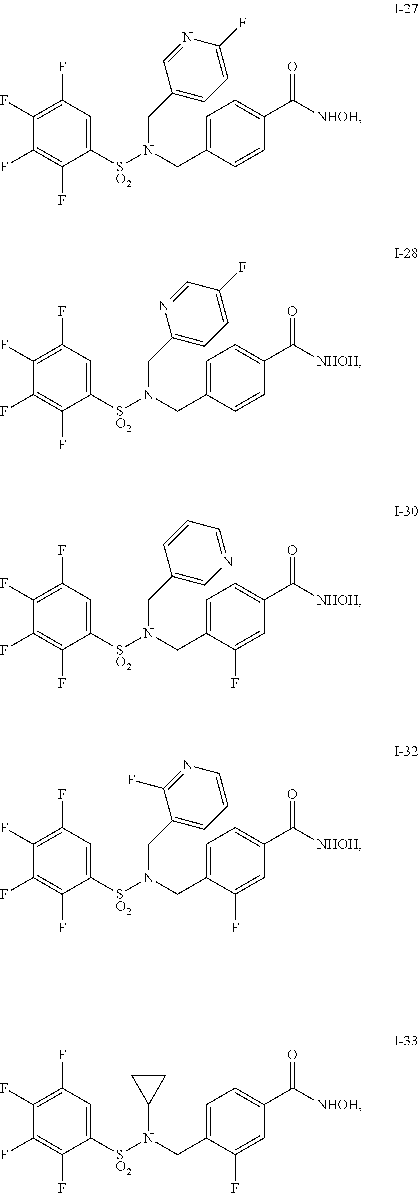

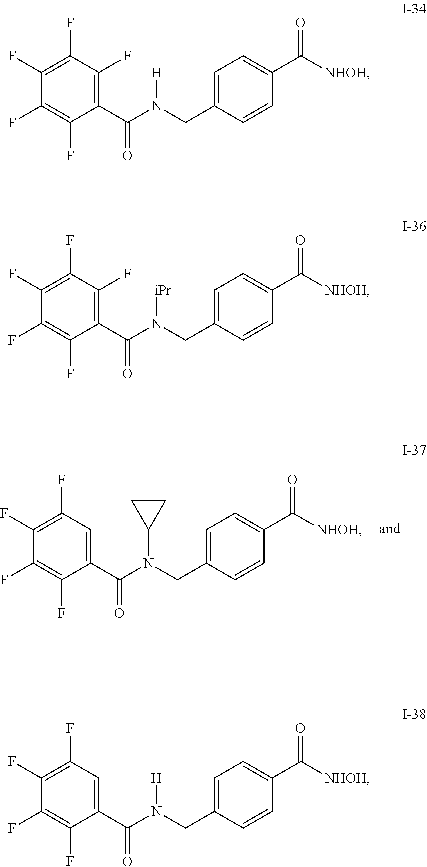

or a pharmaceutically acceptable salt, solvate, and/or prodrug thereof.

In some embodiments, the compound of Formula I is I-3, I-18 or I-19 or pharmaceutically acceptable salt and/or solvate thereof.

The present application also includes a compound of Formula I, wherein the compound of Formula I is a compound of Formula I-B, or a pharmaceutically acceptable salt and/or solvate thereof:

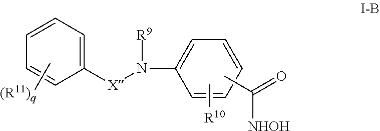

wherein
q is 4 or 5;
X″ is selected from C(O) and SO₂;
R⁹ is selected from C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, each of which is optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₁₋₆cycloalkyl, C₃₋₆heterocycloalkyl, phenyl, and C₅₋₆heteroayl are each unsubstituted or substituted with one or more C₁₋₄alkyl or halo;
R¹⁰ is selected from H, halo, C₁₋₄alkyl, and OC₁₋₄alkyl;
each R¹¹ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

In some embodiments, R⁹ is selected from C₁₋₃alkyleneheteroaryl, C₁₋₆cycloalkyl and C₁₋₃alkylenearyl, each of which is optionally substituted with one or more groups independently selected from halo, C₁₋₄alkyl, N(C₁₋₄alkyl)(C₁₋₄alkyl), OC₁₋₄alkyl, C₁₋₆cycloalkyl, C₃₋₆heterocycloalkyl, phenyl, C₅₋₆heteroayl, in which groups C₃₋₆cycloalkyl, C₁₋₆heterocycloalkyl, phenyl, and C₅₋₆heteroayl are each unsubstituted or substituted with one or more C₁₋₄alkyl or halo.

In some embodiments, R⁹ is selected from C₃cycloalkyl, C₁₋₂alkyleneheteroaryl, and C₁₋₂alkylenearyl, each of which is optionally substituted with one or more groups independently selected from halo, and C₁₋₃alkyl.

In some embodiments, R⁹ is selected from cyclopropyl, benzyl, and pyridinylmethyl, each of which is optionally substituted with one or more groups selected from fluorine and C₁₋₂alkyl. In some embodiments, R⁹ is pyridinylmethyl.

In another aspect, R⁹ is selected from H, and halo.

In some embodiments, R¹⁰ is selected from H, and fluorine.

In some embodiments, X″ is SO₂. In other embodiments, X″ is C(O).

In some embodiments, each R¹¹ is selected from F and Cl. In some embodiments, each R¹¹ is F. In some embodiments, one R¹¹ is Cl and the remaining R¹¹ are F. In some embodiments, two R¹¹ are Cl and the remaining R¹¹ are F.

In some embodiments, the C(O)NHOH group in the compounds of Formula I-B is bonded to the para position of the phenyl ring.

In some embodiments, the present application includes a compound of Formula I wherein the compound of Formula I is a compound of Formula I-C or a pharmaceutically acceptable salt, solvate, and/or prodrug thereof:

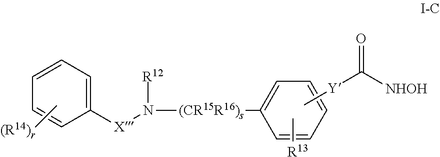

wherein
r is 4 or 5;
s is 0, 1, 2, 3, or 4;
X′″ is selected from C(O) and SO₂;
R¹² is selected from H, C₁₋₁₀alkyl, C₃₋₁₀cycloalkyl, C₁₋₆alkyleneC₃₋₁₀cycloalkyl, C₁₋₆ alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R¹³ is selected from H, halo, C₁₋₄alkyl, and OC₁₋₄alkyl;
each R¹⁴ is the same or different and is selected from halo;
R¹⁵ and R¹⁶ are independently selected from H and C₁₋₄alkyl;
Y′ is selected from C₁₋₆alkyene, C₂₋₆alkenylene and C₂₋₆alkynylene;
the —Y′—C(O)NHOH group is bonded to a meta or para position of the phenyl ring;
all alkyl and alkylene are optionally fluoro substituted; and
all available hydrogen atoms are optionally replaced with deuterium.

In some embodiments, R¹² is selected from H, C₁₋₅alkyl, C₁₋₃alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆ cycloalkyl, C₃₋₆heterocycloalkyl, phenyl, C₅₋₆heteroayl, in which groups C₃₋₆cycloalkyl, C₃₋₆ heterocycloalkyl, phenyl, and C₅₋₆heteroayl are each unsubstituted or substituted with one or more C₁₋₄alkyl or halo.

In some embodiments, R¹² is selected from H, C₁₋₃alkyl, C₃₋₆cycloalkyl, C₁₋₂ alkyleneheteroaryl, and C₁₋₂alkylenearyl, the latter 4 groups optionally substituted with one or more groups independently selected from halo, and C₁₋₃alkyl. In some embodiments, R¹² is selected from H, methyl, isopropyl, C₃₋₆cycloalkyl, benzyl, and pyridinylmethyl, the latter 2 groups are optionally substituted with one or more groups selected from fluorine and C₁₋₂alkyl. In some embodiments, R¹² is pyridinylmethyl. In some embodiments, R¹² is C₃₋₆cycloalkyl. In some embodiments, R¹² is isopropyl or cyclopentyl.

In another aspect, R¹³ is selected from H, and halo.

In some embodiments, R¹³ is selected from H, and fluorine.

In some embodiments, X′″ is SO₂. In other embodiments, X′″ is C(O).

In some embodiments, each R¹⁴ is selected from F and Cl. In some embodiments, each R¹⁴ is F. In some embodiments, one R³ is Cl and the remaining R¹⁴ are F. In some embodiments, two R¹⁴ are Cl and the remaining R¹⁴ are F.

In some embodiments, R¹⁵ and R¹⁶ are independently selected from H and CH₃. In some embodiments, both R¹⁵ and R¹⁶ are H.

In some embodiments, s is 0, 1 or 2. In some embodiments, s is 0 or 1. In some embodiments, s is 1.

In some embodiments, Y′ is selected from C₁₋₄alkyene, C₂₋₄alkenylene and C₂₋₄alkynylene. In some embodiments, Y′ is selected from C₁₋₄alkyene and C₂₋₄alkenylene. In some embodiments, Y′ is selected from —CH₂—, —CH₂CH₂— and —CH═CH—. In some embodiments, Y is —CH═CH—. In some embodiments, Y′ is —CH═CH— and the double bond is in the trans configuration.

In some embodiments, the —Y′—C(O)NHOH group is bonded to the para position of the phenyl ring.

In some embodiments, the compound of Formula I of the present application is a compound of Formula I-C selected from:

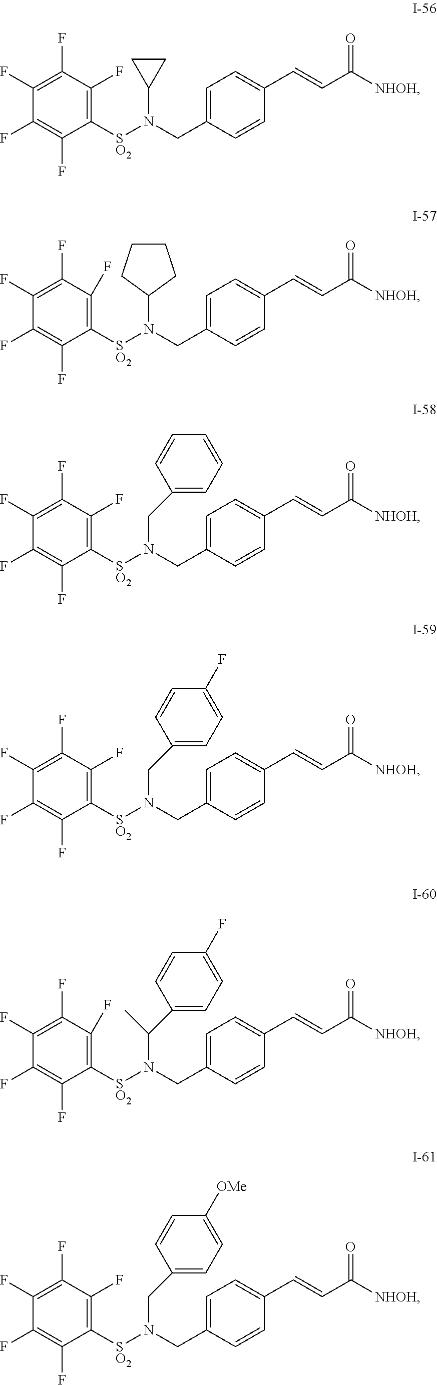

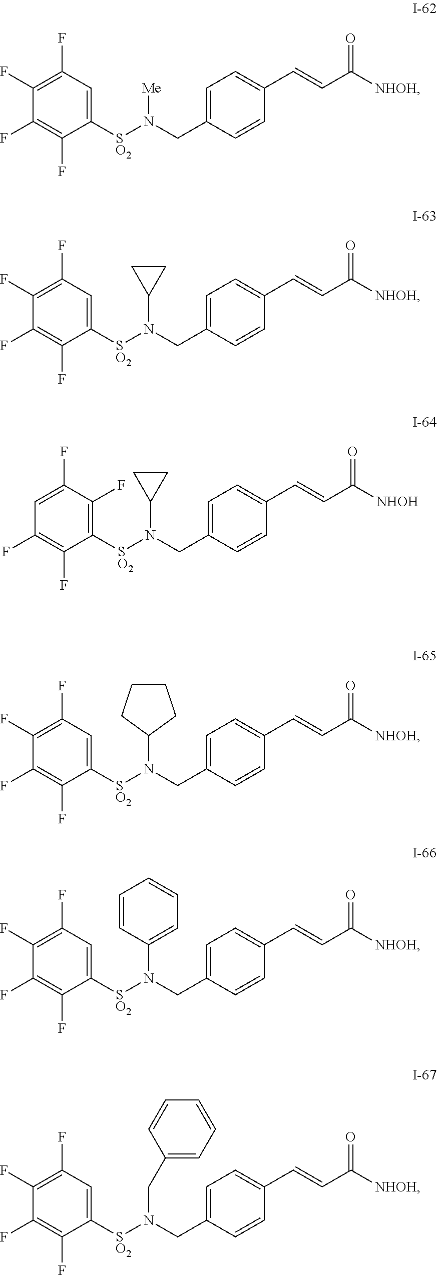

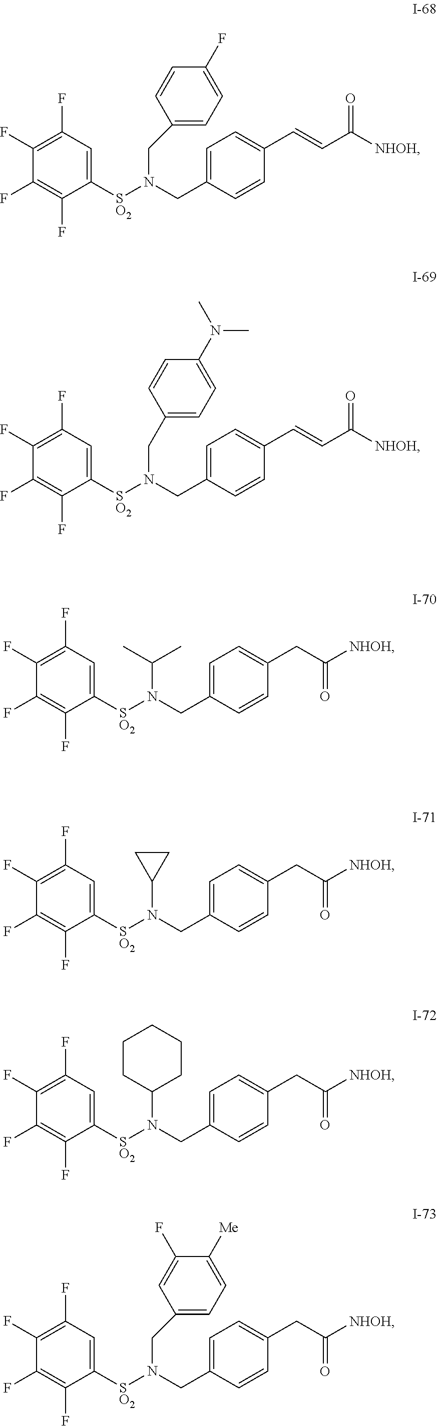

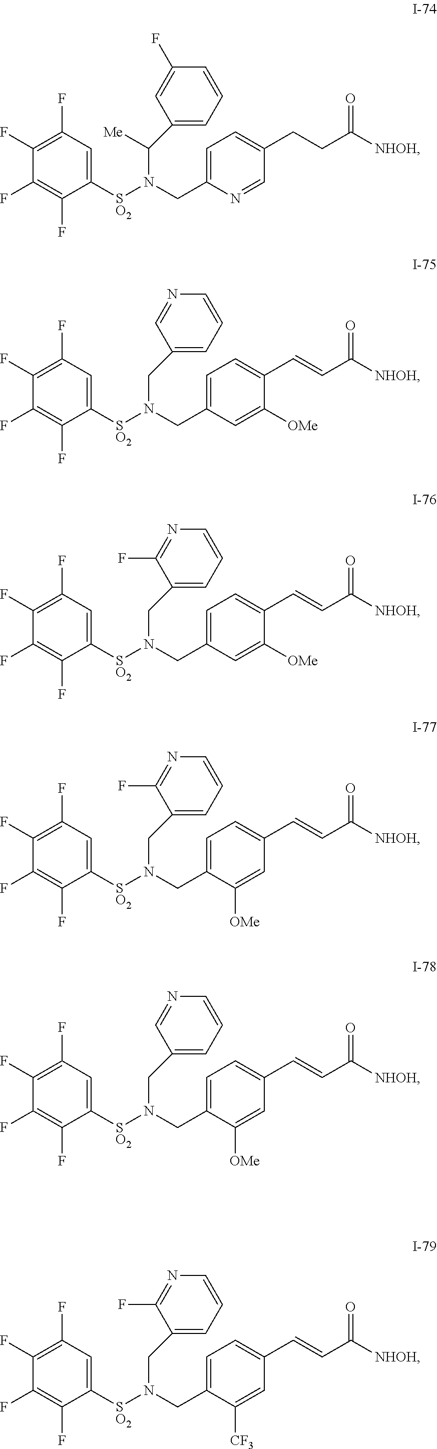

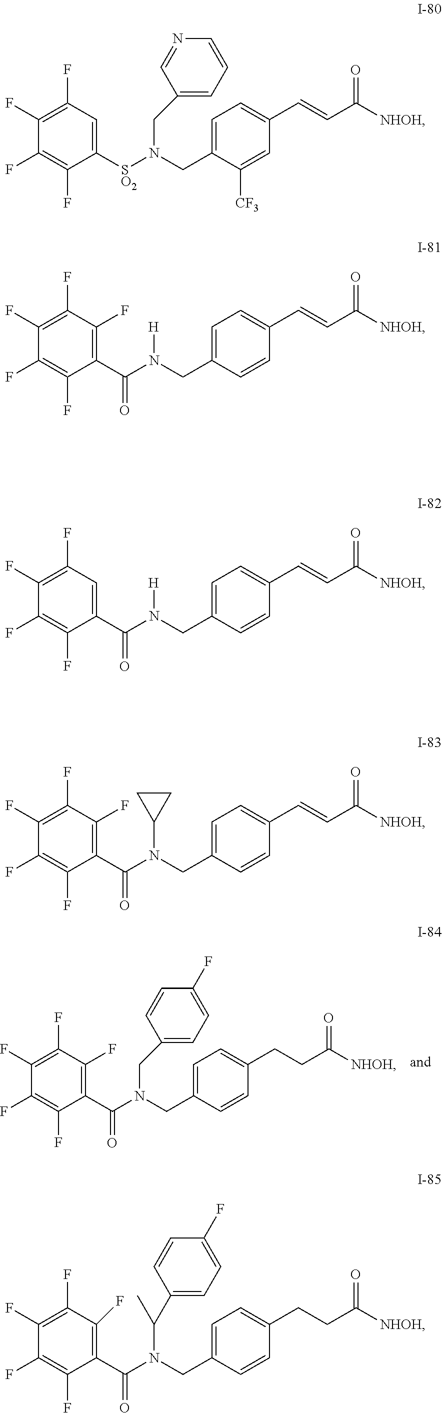

or a pharmaceutically acceptable salt, solvate and/or prodrug thereof.

In some embodiments, the compound of Formula I is a compound of Formula I-C selected from

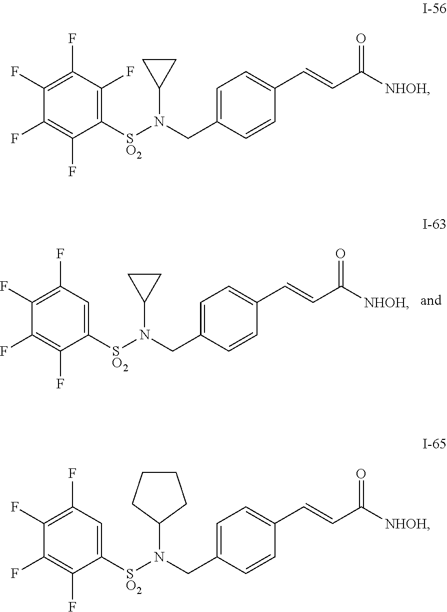

or a pharmaceutically acceptable salt, solvate, and/or prodrug thereof.

In some embodiments, the compound of Formula I-C is pharmaceutically acceptable salt and/or solvate thereof.

In some embodiments, the compounds of the application exist as solvates. Examples of suitable solvents for the solvates, include, but are not limited to ethanol, water and the like. When water is the solvent, the molecule is referred to as a “hydrate”.

In some embodiments, the compounds of the application are acid addition salts. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

In some embodiments, the compounds of the application are basic addition salts. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

Prodrugs of the compounds of the present application may be, for example, conventional esters formed with available hydroxamic acid, hydroxy, thiol, amino and/or carboxylic acid groups. Some common esters which have been utilized as prodrugs are phenyl esters, aliphatic (C₁-C₂₄) esters, acyloxymethyl esters, carbamates and amino acid esters.

In some embodiments, the compounds of the application are selective inhibitors of HDAC6. In some embodiments, the compounds of the application have an IC₅₀ for HDAC6 that is 2 times, 3 times, 5 times, 10 times, 20 times, 30 times, 50 times, or 100 times lower than the IC₅₀ value of that compound for at least one other member of the HDAC family. In some embodiments, the compounds of the application have an IC₅₀ for HDAC6 that is 2 times, 3 times, 5 times, 10 times, 20 times, 30 times, 50 times, and/or 100 times lower than the IC₅₀ value of that compound for all other members of the HDAC family.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

Depending on the mode of administration, in some embodiments, the pharmaceutical composition may comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of a pharmaceutically acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16^{th} Ed., Mack Publishing, Easton, Pa., 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

To be clear, in the above, the term “a compound” also includes embodiments wherein one or more compounds are referenced.

### III. Methods and Uses of the Application

Compounds of the present application show selective inhibition of HDAC6 and strong anticancer activity. Accordingly the present application includes the use of one or more compounds of the application or a composition of the application, as a medicament.

Accordingly, the present application includes a method of selectively inhibiting HDAC6 in a cell comprising administering an effective amount of one or more compounds of the present application or one or more compositions of the present application in the cell.

The application also includes a use of one more compounds of the application, or one or more compositions of the present application, for selectively inhibiting HDAC6 in a cell as well as a use of one or more compounds of the application, or one or more compositions of the present application, for the preparation of a medicament for selectively inhibiting HDAC6 in a cell. The application further includes one or more compounds of the application, or one or more compositions of the present application, for use in selectively inhibiting HDAC6.

As the compounds of the application have been shown to selectively inhibit HDAC6, the compounds of the application are useful for treating a disease, disorder or condition that benefits from inhibiting HDAC6.

Accordingly, in another aspect, the present application includes a method of treating a disease, disorder or condition that benefits from inhibiting HDAC6 comprising administering an effective amount of one or more compounds of the present application, or one or more compositions of the present application, to a subject in need thereof.

The application also includes a use of one more compounds of the application, or one or more compositions of the present application, for treating a disease, disorder or condition that benefits from inhibiting HDAC6 and use of one more compounds of the application, or one or more compositions of the present application, for preparation of a medicament for treating a disease, disorder or condition that benefits from inhibiting HDAC6. Also included is use of one more compounds of the application, or one or more compositions of the present application, for use to treat a disease, disorder or condition that benefits from inhibiting HDAC6.

In some embodiments, the diseases, disorders or conditions that benefit from inhibiting HDAC6 are diseases, disorders or conditions that benefit from selectively inhibiting HDAC6. In some embodiments, the disease, disorder or condition that benefits from selectively inhibiting HDAC6 is a cell proliferative disorder. In some embodiments, the disease, disorder or condition that benefits from inhibiting HDAC6 is cancer.

In some embodiments, the cancer includes, but is not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In some embodiments, the cancer is hematological cancer or brain cancer. In some embodiments, the cancer is leukemia, adenosarcoma, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system or lymphoma. In some embodiments, the leukemia is acute myeloid leukemia, acute lymphoblastic leukemia (ALL), or chronic myeloid leukemia (CML).

In some embodiments, the cancer is breast cancer, multiple myeloma, pancreatic cancer, lung cancer, prostate cancer, renal cancer, ovarian cancer and leukemias such as acute myeloid leukemia (AML) and acute lymphoblastic leukemia (ALL).

In some embodiments, the disease, disorder or condition that benefits from inhibiting HDAC6 is selected from a cardiovascular disease, a bacterial infection, a neurological disease, inflammation and immunological disorders such as rheumatoid arthritis, psoriasis, multiple sclerosis, lupus and organ transplant rejection.

In some embodiments, the compounds or compositions of the application are used in combination with other active agents. In some embodiments, the compounds of the application may be used alone or in combination with other known active agents useful for treating diseases, disorders or conditions that benefit from inhibiting HDAC6, or that are treatable by inhibition of HDAC6. In some embodiments, the other active agents are selected from one or more of chemotherapeutics, microtubule destabilizing agents, Hsp90 inhibitors, inhibitors of Hsp90 downstream proteins, tyrosine kinase inhibitors, HER-2 inhibitors, BCR-ABL inhibitors, Akt inhibitors, c-Raf and MEK inhibitors, Aurora A and B inhibitors, EGFR inhibitors, proteasome inhibitors, ubiquitin proteasome system inhibitors, modulators of autophagy and protein homeostasis agents.

When used in combination with other active agents it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another active agent simultaneously or sequentially in separate unit dosage forms or together in a single unit dosage form. Accordingly, the present application provides a single unit dosage form comprising one or more compounds of the application (e.g. a compound of Formula I), an additional active agent, and a pharmaceutically acceptable carrier.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art. For example, in the context of treating a cell proliferative disorder, an effective amount is an amount that, decreases said cell proliferative disorder compared to the decrease without administration of the one or more compounds or compositions of the application.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of compound per tablet. Compounds of the application may be administered in a single daily, weekly or monthly dose or the total daily dose may be divided into two, three or four daily doses.

In some embodiments, the cell is in vivo or in vitro.

In some embodiments, the subject is a mammal. In some embodiments, the subject is human.

To be clear, in the above, the term “a compound” also includes embodiments wherein one or more compounds are referenced.

### IV. Methods of Making Compounds of the Application

In some embodiments, compounds of Formula I are prepared as shown in Scheme 1. Therefore compounds of Formula B, wherein R^{a} is a suitable protecting group (e.g. benzyl or tetrahydropyran-2-yl) and R¹-R³, n and X are as defined in Formula I, are treated under conditions to remove the protecting group to provide compounds of Formula I. In some embodiments, compounds of Formula B are prepared by activating the carboxylic acid of compounds A, for example using an amino acid coupling reagent or by conversion to the corresponding acid chloride, followed by addition of a compound of the formula R^{a}ONH₂, wherein R^{a} is the suitable protecting group, in the presence of a suitable base.

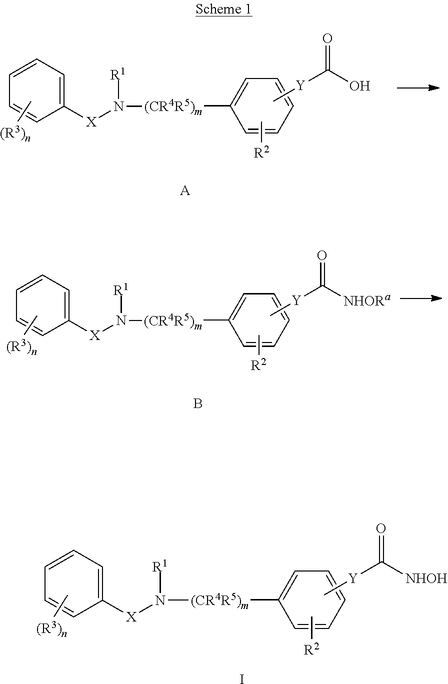

In some embodiments, compounds of Formula A are prepared as shown in Scheme 2 by reacting a compound of Formula C, wherein LG is a suitable leaving group, such as chlorine, with an amine of Formula D, wherein R^{b} is hydrogen or a suitable protecting group, in a solvent such as dichloromethane (DCM) or chloroform in the presence of a base such as N,N-diisopropylethylamine (DIPEA) or triethylamine (TEA) to provide compounds of Formula A wherein R¹ is H. In some embodiments, this reaction is carried out at 0° C., and is slowly warmed to an ambient temperature. The desired product of this reaction (a compound of Formula A wherein R¹ is H) is then reacted with an appropriate reagent of Formula D, in which LG′ is a suitable leaving group such as bromide, in a solvent such as dimethylforamide (DMF) and in the presence of a base such as DIPEA or TEA to yield, after removal of any protecting groups if needed, the compounds with the generic structure A wherein R¹ is other than H.

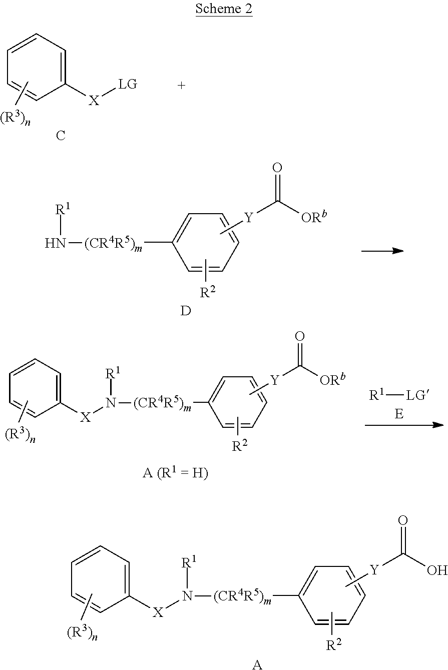

Alternatively, as shown in Scheme 3, compounds with the generic structure A, wherein R¹ is other than H, may also be prepared by reacting appropriate starting amine (F) in a solvent such as 1,2-dichloroethane (DCE) with an appropriate aldehyde (G), wherein R^{b} is hydrogen or a suitable protecting group, and, for example, sodium triacetoxyborohydride. In some embodiments, this reaction is carried out at ambient temperature. The desired secondary amine product (H) is then reacted in a solvent such as dichloromethane (DCM) or chloroform in the presence of a base such as N,N-diisopropylethylamine or triethylamine and phenylsulfonyl chloride (C), to provide, after removal of any protecting groups as needed, compounds of Formula A. In some embodiments, this reaction is carried out at 0° C., and slowly warmed to an ambient temperature.

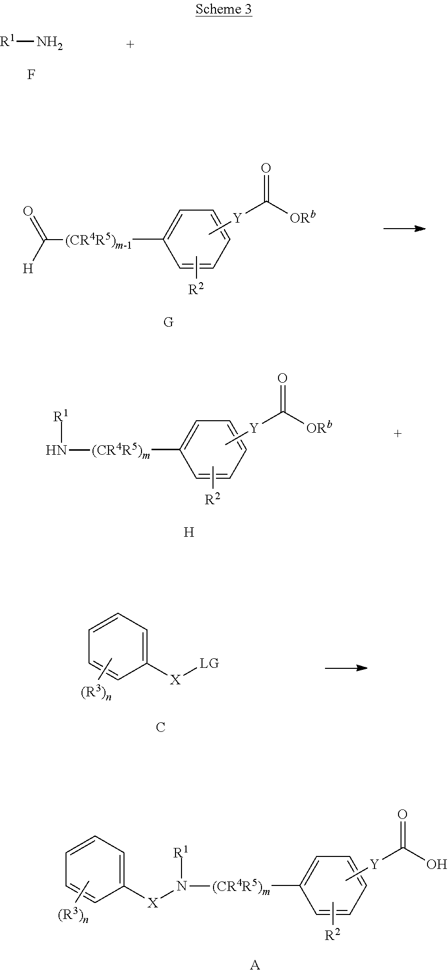

In another alternative, as shown in Scheme 4, compounds with the generic structure A wherein R¹ is other than H are prepared as shown in Scheme 4 by reacting compounds of Formula C in a solvent such as dichloromethane (DCM) or chloroform in the presence of a base such as N,N-diisopropylethylamine (DIPEA) or triethylamine (TEA) and appropriate amine of Formula E. In some embodiments, this reaction is carried out at 0° C., and is slowly warmed to an ambient temperature. The desired product of this reaction (compounds of Formula J) is then reacted with an appropriate reagent of Formula K, in which LG” is a suitable leaving group such as bromide and R^{b} is hydrogen or a suitable protecting group, in a solvent such as dimethylforamide (DMF) and in the presence of a base such as cesium carbonate to yield, after removal of any protecting groups as needed, the compounds of Formula A, wherein R¹ is other than H.

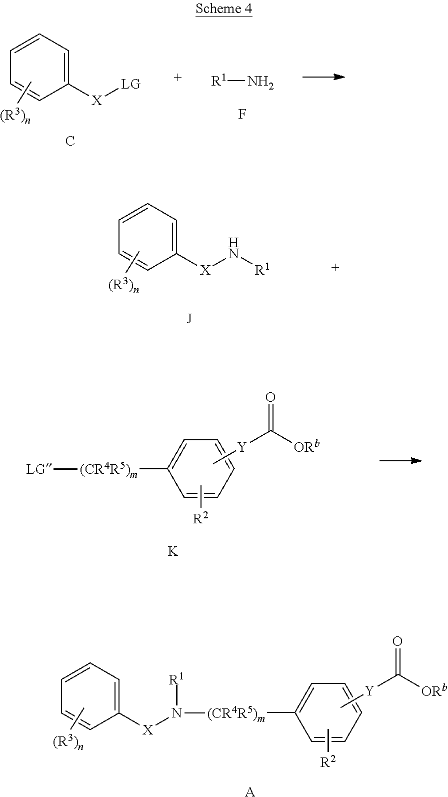

Generally the reactions described above are performed in a suitable inert organic solvent and at temperatures and for times that will optimize the yield of the desired compounds. Examples of suitable inert organic solvents include, but are not limited to, dimethylformamide (DMF), dioxane, methylene chloride, chloroform, tetrahydrofuran (THF), toluene, and the like.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### I. Examples

The following non-limiting examples are illustrative of the present application:

A. General Methods

Exemplary compounds of the application were synthesized using the methods described herein, or other methods, which are known in the art. Unless otherwise noted, reagents and solvents were obtained from commercial suppliers.

Anhydrous solvents methanol, dichloromethane (CH₂Cl₂, DCM), tetrahydrofuran (THF) and dimethylformamide (DMF) were purchased from Sigma Aldrich and used directly from Sure-Seal bottles. All reactions were performed under an atmosphere of dry nitrogen in oven-dried glassware and were monitored for completeness by thin-layer chromatography (TLC) using silica gel (visualized by UV light, or developed by treatment with KMnO₄ stain). NMR spectra were recorded in Bruker Avance III spectrometer at 23° C., operating at 400 MHz for ¹H NMR and 100 MHz ¹³C NMR spectroscopy either in CDCl₃, CD₃OD or d₆-DMSO. Chemical shifts (d) are reported in parts per million (ppm) after calibration to residual isotopic solvent. Coupling constants (J) are reported in Hz. Mass spectrometry was performed with an AB/Sciex QStar mass spectrometer with an ESI source, MS/MS and accurate mass capabilities, associated with an Agilent 1100 capillary LC system. Before biological testing, inhibitor purity was evaluated by reversed-phase HPLC (rpHPLC). Analysis by rpHPLC was performed using a Phenomenex Luna 5u C18 150 mm×4.6 mm column run at 1.2 mL/min, and using gradient mixtures. The linear gradient consisted of a changing solvent composition of either (I) 15% MeCN and 85% H₂O with 0.1% TFA (v/v) to 100% MeCN over 30 minutes and (II) 15% MeCN and 85% H₂O with 0.1% TFA (v/v) to 100% MeCN over 60 minutes, UV detection at 250 nm. For reporting HPLC data, percentage purity is given in parentheses after the retention time for each condition. All biologically evaluated compounds are >95% chemical purity as measured by HPLC. The HPLC traces for all tested compounds are provided in supporting information.

B. Synthesis of Compounds

### Synthesis of 4-((N-(4-(tert-butyl)benzyl)-2,3,4,5,6-pentafluorophenylsulfonamido)methyl)-N-hydroxybenzamide (Compound I-8)

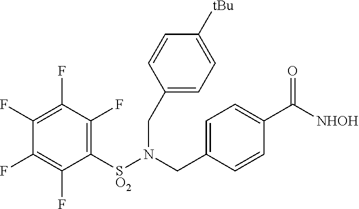

General Procedure 1:

To the vacuum-dried and nitrogen-purged precursor, (benzylhydroxamate ester, 1 eq) in 2:1 THF:MeOH was added 0.1 eq. 10% palladium on carbon and mixture was purged 3 times with nitrogen. Hydrogen gas was then bubbled into mixture for 3 hrs or until TLC confirms complete disappearance of starting ester material. Solution was filtered through celite, and concentrated on the rotary evaporator.

Preparative high performance liquid chromatography (prep-HPLC) was used to purify product using mobile phase gradient of 5%-100% acetonitrile in water containing 0.1% formic acid. Pure fraction was confirmed by low-resolution mass spectrometry (LRMS), and purity was checked on analytical HPLC using similar conditions as above. Fractions with purity over 95% were combined and lyophilized to give the product as a white solid.

NMR was collected on a 400 MHz Bruker NMR using acetonitrile d₃. Chemical shifts in ppm recorded below.

1H NMR (400 MHz, Acetonitrile-d3) δ 9.76 (s, 1H), 7.69 (d, J=8.1 Hz, 1H), 7.64-7.58 (m, 2H), 7.28 (d, J=8.2 Hz, 2H), 7.21 (d, J=8.0 Hz, 1H), 6.93 (td, J=8.3, 6.5 Hz, 2H), 4.57 (d, J=9.6 Hz, 2H), 4.52 (s, 2H), 1.94 (s, 9H). 19F NMR (377 MHz, Acetonitrile-d3) δ −136.33 (ddd, J=19.7, 14.5, 8.5 Hz), −148.39 (dtt, J=33.0, 20.2, 6.6 Hz), −163.76 (qt, J=20.6, 6.7 Hz).

### Synthesis of 4-((N-benzyl-2,3,4,5,6-pentafluorophenylsulfonamido)methyl)-N-hydroxybenzamide (Compound I-5)

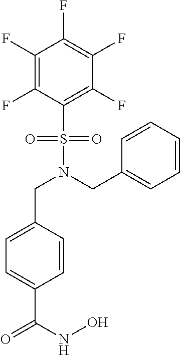 [SMILES: O=C(NO)c1ccc(CN(Cc2ccccc2)S(=O)(=O)c2c(F)c(F)c(F)c(F)c2F)cc1]

Compound I-5 was synthesized using General Procedure I as for I-8.

¹H NMR (400 MHz, Acetonitrile-d₃) δ 9.71 (s, 1H), 7.70 (d, J=7.9 Hz, 1H), 7.64 (d, J=8.2 Hz, 2H), 7.28 (d, J=7.2 Hz, 4H), 7.21 (d, J=8.4 Hz, 2H), 4.64-4.54 (m, 4H). ¹⁹F NMR (376 MHz, Acetonitrile-d₃) δ −137.21 (t, J=24.1 Hz), −149.05 (d, J=56.5 Hz), −161.13-−161.50 (m).

### Synthesis of N-hydroxy-4-((2,3,4,5,6-pentafluoro-N-(4-methylbenzyl)phenylsulfonamido)methyl)benzamide (Compound I-6)

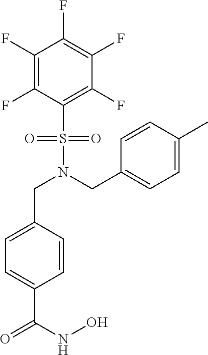 [SMILES: Cc1ccc(CN(Cc2ccc(C(=O)NO)cc2)S(=O)(=O)c2c(F)c(F)c(F)c(F)c2F)cc1]

Compound I-6 was synthesized using General Procedure 1, similar to I-8.

¹H NMR (400 MHz, Acetonitrile-d3) δ 9.72 (s, 1H), 7.69-7.61 (m, 2H), 7.31 (d, J=8.2 Hz, 2H), 7.10 (d, J=6.7 Hz, 4H), 4.60 (d, J=13.5 Hz, 2H), 4.50 (s, 2H), 2.29 (s, 3H). ¹⁹F NMR (377 MHz, Acetonitrile-d3) δ −137.16 (qt, J=15.7, 7.7 Hz), −149.11-−149.43 (m), −161.33-−161.60 (m).

### Synthesis of N-hydroxy-4-((2,3,4,5,6-pentafluoro-N-(4-fluorobenzyl)phenylsulfonamido)methyl)benzamide (Compound I-9)

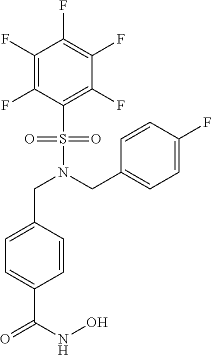 [SMILES: O=C(NO)c1ccc(CN(Cc2ccc(F)cc2)S(=O)(=O)c2c(F)c(F)c(F)c(F)c2F)cc1]

Compound I-9 was synthesized using General Procedure 1, similar to I-8.

¹H NMR (400 MHz, Methanol-d₄) δ 7.64 (d, J=8.1 Hz, 2H), 7.31 (d, J=8.1 Hz, 2H), 7.23 (dd, J=8.4, 5.3 Hz, 2H), 6.95 (t, J=8.7 Hz, 2H), 4.61 (d, J=10.4 Hz, 2H), 4.55 (s, 2H). ¹⁹F NMR (376 MHz, Methanol-d₄) δ −117.68 (d, J=9.4 Hz), −139.21 (d, J=21.3 Hz), −151.24 (d, J=20.0 Hz), −163.60-−163.84 (m).

### Synthesis of N-hydroxy-4-((2,3,4,5,6-pentafluoro-N-(4-(trifluoromethyl)benzyl)phenylsulfonamido)methyl)benzamide Compound I-10

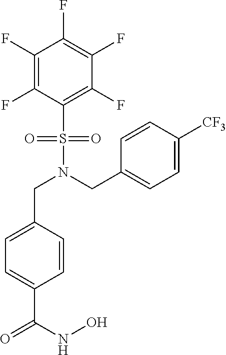 [SMILES: O=C(NO)c1ccc(CN(Cc2ccc(C(F)(F)F)cc2)S(=O)(=O)c2c(F)c(F)c(F)c(F)c2F)cc1]

Compound I-10 was synthesized using General Procedure 1.

¹H NMR (400 MHz, DMSO-d6) δ 11.18 (s, 1H), 9.04 (s, 1H), 7.67-7.50 (m, 4H), 7.42 (d, J=8.0 Hz, 2H), 7.28 (d, J=8.0 Hz, 2H), 4.65 (d, J=3.4 Hz, 4H). ¹⁹F NMR (376 MHz, DMSO-d6) δ −61.09 (d, J=6.6 Hz), −136.26 (d, J=24.8 Hz), −147.06 (s), −159.55-−159.80 (m).

### Synthesis of N-hydroxy-4-((2,3,4,5,6-pentafluoro-N-(3,4,5-trifluorobenzyl)phenylsulfonamido)methyl)benzamide (Compound I-11)

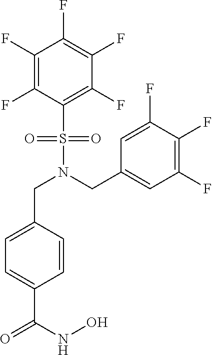 [SMILES: O=C(NO)c1ccc(CN(Cc2cc(F)c(F)c(F)c2)S(=O)(=O)c2c(F)c(F)c(F)c(F)c2F)cc1]

¹H NMR (400 MHz, Acetonitrile-d₃) δ 9.76 (s, 1H), 7.64-7.58 (d, J=8.1, 2H), 7.29-7.20 (d, J=8.2 Hz, 2H), 6.93 (td, J=8.3, 6.5 Hz, 2H), 4.57 (d, J=9.6 Hz, 2H), 4.52 (s, 2H). ¹⁹F NMR (377 MHz, Acetonitrile-d₃) δ −136.33 (ddd, J=19.6, 14.5, 8.4 Hz), −137.06-−137.23 (m), −148.39 (dtt, J=33.1, 20.1, 6.6 Hz), −160.86-−161.10 (m), −163.76 (qt, J=20.6, 6.6 Hz).

### Synthesis of 4-((N-benzyl-2,3,4,5-tetrafluorophenylsulfonamido)methyl)-N-hydroxybenzamide (Compound I-16)

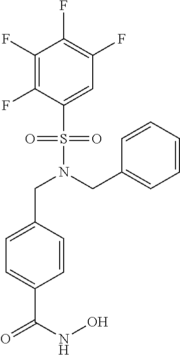 [SMILES: O=C(NO)c1ccc(CN(Cc2ccccc2)S(=O)(=O)c2cc(F)c(F)c(F)c2F)cc1]

Compound I-10 was synthesized using General Procedure 1.

¹H NMR (400 MHz, Acetonitrile-d₃) δ 9.84 (s, 1H), 7.63 (d, J=7.9 Hz, 2H), 7.56 (dddd, J=10.1, 8.2, 5.9, 2.5 Hz, 1H), 7.26 (dd, J=6.8, 2.3 Hz, 5H), 7.15 (dd, J=6.7, 2.9 Hz, 2H), 4.54 (s, 2H), 4.48 (s, 2H). ¹⁹F NMR (376 MHz, Acetonitrile-d₃) δ −135.31 (ddt, J=20.7, 13.5, 7.1 Hz), −138.17-−138.35 (m), −149.24 (ddd, J=27.8, 19.1, 8.5 Hz), −153.77-−153.96 (m). ¹³C NMR (101 MHz, Acetonitrile-d₃) δ 139.86, 135.32, 131.04, 128.61, 128.48, 128.44, 127.90, 127.04, 112.76, 112.72, 112.50, 52.00, 51.98, 51.44, 51.41.

### Synthesis of N-hydroxy-4-((2,3,4,5-tetrafluoro-N-(pyridin-4-ylmethyl)phenylsulfonamido)methyl)benzamide (Compound I-17)

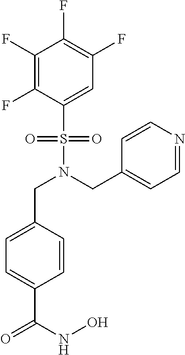 [SMILES: O=C(NO)c1ccc(CN(Cc2ccncc2)S(=O)(=O)c2cc(F)c(F)c(F)c2F)cc1]

Compound I-17 was synthesized using General Procedure 1.

¹H NMR (400 MHz, Acetonitrile-d₃) δ 7.70-7.57 (m, 3H), 7.26 (d, J=7.5 Hz, 2H), 7.17 (dd, J=14.4, 6.5 Hz, 4H), 4.54 (s, 2H), 4.51 (s, 2H). ¹⁹F NMR (376 MHz, Acetonitrile-d₃) δ −134.86-−135.14 (m), −138.00-−138.23 (m), −148.63-−148.96 (m), −153.43-−153.67 (m). ¹³C NMR (101 MHz, Acetonitrile-d₃) δ 166.30, 161.79, 150.13, 146.33, 139.52, 136.97, 133.04, 129.55, 127.82, 126.02, 123.75, 115.18, 53.08, 51.62

### Synthesis of N-hydroxy-4-((2,3,4,5-tetrafluoro-N-(pyridin-3-ylmethyl)phenylsulfonamido)methyl)benzamide (Compound I-18)

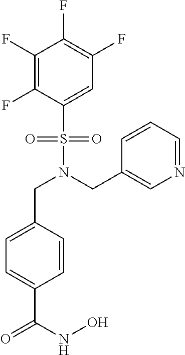 [SMILES: O=C(NO)c1ccc(CN(Cc2cccnc2)S(=O)(=O)c2cc(F)c(F)c(F)c2F)cc1]

Compound I-18 was synthesized using General Procedure 1.

¹H NMR (400 MHz, Acetonitrile-d₃) δ 8.42 (s, 1H), 8.34-8.29 (m, 1H), 7.63 (dq, J=17.5, 7.2, 5.8 Hz, 4H), 7.26 (d, J=8.3 Hz, 2H), 7.21-7.15 (m, 1H), 4.57-4.44 (m, 4H). ¹⁹F NMR (376 MHz, Acetonitrile-d₃) δ −135.00-−135.14 (m), −138.08-−138.24 (m), −148.77-−149.03 (m), −153.59 (t, J=20.1 Hz).

### Synthesis of N-hydroxy-4-((2,3,4,5-tetrafluoro-N-((2-fluoropyridin-3-yl)methyl)phenylsulfonamido)methyl)benzamide (Compound I-19)

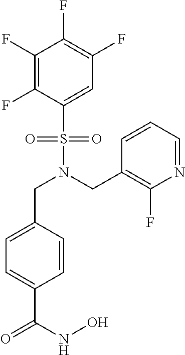 [SMILES: O=C(NO)c1ccc(CN(Cc2cccnc2F)S(=O)(=O)c2cc(F)c(F)c(F)c2F)cc1]

Compound I-19 was synthesized using General Procedure 1.

¹H NMR (400 MHz, Acetonitrile-d₃) δ 9.84 (s, 1H), 8.00 (dt, J=4.8, 1.6 Hz, 1H), 7.75-7.55 (m, 4H), 7.29 (d, J=8.0 Hz, 2H), 7.12 (ddd, J=7.1, 4.8, 1.9 Hz, 1H), 4.57 (s, 2H), 4.53 (s, 2H). ¹⁹F NMR (376 MHz, Acetonitrile-d₃) δ −73.19 (d, J=9.9 Hz), −134.99 (ddt, J=21.0, 13.9, 7.2 Hz), −13C NMR (101 MHz, Acetonitrile-d₃) δ 164.57, 161.18, 149.59, 143.19, 140.40, 132.27, 130.16, 128.21, 123.92, 119.70, 114.51, 54.20, 48.03

### Synthesis of N-hydroxy-4-((2,3,4,5-tetrafluoro-N-(pyridin-2-ylmethyl)phenylsulfonamido)methyl)benzamide (Compound I-20)

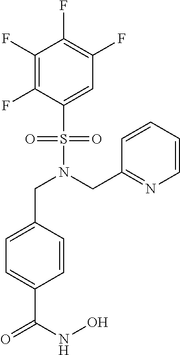 [SMILES: O=C(NO)c1ccc(CN(Cc2ccccn2)S(=O)(=O)c2cc(F)c(F)c(F)c2F)cc1]

Compound I-20 was synthesized using General Procedure 1.

¹H NMR (400 MHz, Acetonitrile-d₃) δ 9.81 (s, 1H), 8.33 (d, J=4.5 Hz, 1H), 7.68 (s, 1H), 7.68-7.54 (m, 4H), 7.33 (d, J=7.7 Hz, 2H), 7.22-7.12 (m, 2H), 4.67 (s, 2H), 4.55 (s, 2H). ¹⁹F NMR (376 MHz, Acetonitrile-d₃) δ −134.56 (dq, J=13.8, 6.7 Hz), −138.82-−139.02 (m), −149.73 (tt, J=19.2, 8.1 Hz), −154.60-−154.79 (m). ¹³C NMR (101 MHz, Acetonitrile-d₃) δ 155.14, 149.12, 139.90, 136.70, 128.48, 127.13, 122.85, 122.73, 112.40, 52.24, 51.58.

### Synthesis of N-hydroxy-4-((2,3,4,5-tetrafluoro-N-(2-fluorophenethyl)phenylsulfonamido)methyl)benzamide (Compound I-21)

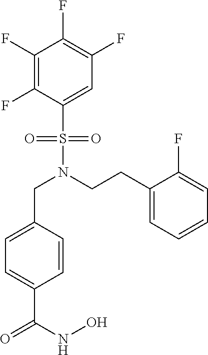 [SMILES: O=C(NO)c1ccc(CN(CCc2ccccc2F)S(=O)(=O)c2cc(F)c(F)c(F)c2F)cc1]

Compound I-21 was synthesized using General Procedure 1.

¹H NMR (400 MHz, Acetonitrile-d₃) δ 9.64 (s, 1H), 7.79 (dd, J=25.2, 8.0 Hz, 2H), 7.58 (dddd, J=9.6, 8.2, 5.9, 2.6 Hz, 1H), 7.47 (d, J=8.0 Hz, 2H), 7.22 (tdd, J=7.5, 5.3, 1.9 Hz, 1H), 7.13 (td, J=7.6, 1.9 Hz, 1H), 7.05 (td, J=7.4, 1.2 Hz, 1H), 7.04-6.94 (m, 1H), 4.63 (s, 2H), 3.50 (t, J=7.4 Hz, 2H), 2.77 (s, 2H). ¹⁹F NMR (376 MHz, Acetonitrile-d₃) δ −119.83-−119.98 (m), −135.15 (ddp, J=22.9, 16.0, 7.1 Hz), −138.37-−138.60 (m), −149.46 (tt, J=19.9, 8.3 Hz), −153.42-−153.66 (m). ¹³C NMR (101 MHz, Acetonitrile-d₃) δ 140.00, 131.43, 131.32, 131.27, 128.75, 128.67, 128.47, 127.31, 125.63, 124.34, 124.30, 115.11, 114.90, 112.53, 50.75, 47.67, 27.58.

### Synthesis of N-hydroxy-4-((2,3,4,5-tetrafluoro-N-(3-fluorobenzyl)phenylsulfonamido)methyl)benzamide (Compound I-23)

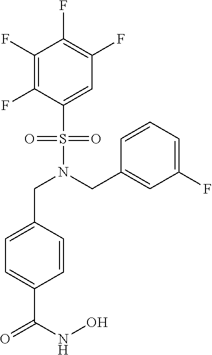 [SMILES: O=C(NO)c1ccc(CN(Cc2cccc(F)c2)S(=O)(=O)c2cc(F)c(F)c(F)c2F)cc1]

Compound I-23 was synthesized using General Procedure 1.

¹H NMR (400 MHz, Acetonitrile-d₃) δ 9.81 (s, 1H), 7.63 (s, 1H), 7.62 (d, J=8.0 Hz, 2H), 7.64-7.54 (m, 1H), 7.32-7.21 (m, 3H), 7.00 (d, J=2.0 Hz, 1H), 7.00-6.87 (m, 2H), 4.54 (s, 2H), 4.49 (s, 2H). ¹⁹F NMR (376 MHz, Acetonitrile-d₃) δ −114.71 (td, J=9.4, 5.9 Hz), −135.08-−135.32 (m), −138.12-−138.29 (m), −148.96-−149.24 (m), −153.68-−153.87 (m). ¹³C NMR (101 MHz, Acetonitrile-d₃) δ 164.12, 167.79, 139.61, 138.43, 131.12, 130.36, 130.28, 128.57, 126.99, 124.40, 124.37, 115.24, 115.03, 114.61, 114.39, 112.82, 112.60, 51.84, 51.81, 51.68.

### Synthesis of N-hydroxy-4-((2,3,4,5-tetrafluoro-N-(2-fluoro-4-methylbenzyl)phenylsulfonamido)methyl)benzamide (Compound I-26)

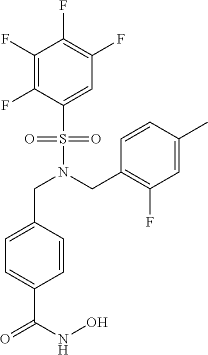 [SMILES: Cc1ccc(CN(Cc2ccc(C(=O)NO)cc2)S(=O)(=O)c2cc(F)c(F)c(F)c2F)c(F)c1]

Compound I-26 was synthesized using General Procedure 1.

¹H NMR (400 MHz, Acetonitrile-d3) δ 9.76 (s, 1H), 7.71 (d, J=7.9 Hz, 1H), 7.63 (d, J=8.2 Hz, 2H), 7.57 (dddd, J=9.7, 8.2, 5.9, 2.5 Hz, 1H), 7.26 (d, J=8.0 Hz, 1H), 7.22-7.07 (m, 2H), 6.92-6.79 (m, 2H), 4.53 (d, J=11.3 Hz, 2H), 4.44 (s, 2H), 2.22 (d, J=6.3 Hz, 3H). ¹⁹F NMR (376 MHz, Acetonitrile-d3) δ −118.90-−119.04 (m), −135.17-−135.43 (m), −138.35 (dtd, J=22.4, 12.7, 11.3, 3.8 Hz), −149.17-−149.47 (m), −153.81-−154.01 (m). ¹³C NMR (101 MHz, Acetonitrile-d3) δ 164.10, 161.98, 159.57, 139.36, 135.93, 132.43, 128.54, 127.33, 125.38, 124.65, 124.08, 115.21, 113.19, 51.78, 14.24.

### Synthesis of N-hydroxy-4-((2,3,4,5-tetrafluoro-N-((6-fluoropyridin-3-yl)methyl)phenylsulfonamido)methyl)benzamide (Compound I-27)

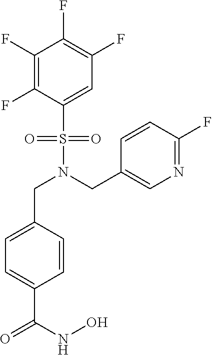 [SMILES: O=C(NO)c1ccc(CN(Cc2ccc(F)nc2)S(=O)(=O)c2cc(F)c(F)c(F)c2F)cc1]

Compound I-27 was synthesized using General Procedure 1.

¹H NMR (400 MHz, Acetonitrile-d₃) δ 9.86 (s, 1H), 7.91 (d, J=2.5 Hz, 1H), 7.68 (dd, J=8.2, 2.6 Hz, 1H), 7.68-7.57 (m, 3H), 7.25 (d, J=8.1 Hz, 2H), 6.84 (dd, J=8.5, 2.8 Hz, 1H), 4.53 (s, 2H), 4.49 (s, 2H). ¹⁹F NMR (376 MHz, Acetonitrile-d₃) δ −71.31 (d, J=7.7 Hz), −135.02 (ddt, J=21.1, 13.9, 7.2 Hz), −137.96-−138.14 (m), −148.74 (ddd, J=27.5, 18.7, 8.1 Hz), −153.46 (td, J=21.2, 19.6, 3.5 Hz). ¹³C NMR (101 MHz, Acetonitrile-d₃) δ 164.21, 161.86, 147.62, 147.47, 142.09, 142.01, 139.59, 131.12, 129.75, 129.71, 128.62, 127.07, 112.91, 112.72, 109.37, 108.99, 52.06, 52.04, 49.14.

### Synthesis of N-hydroxy-4-((2,3,4,5-tetrafluoro-N-((3-fluoropyridin-4-yl)methyl)phenylsulfonamido)methyl)benzamide (Compound I-28)

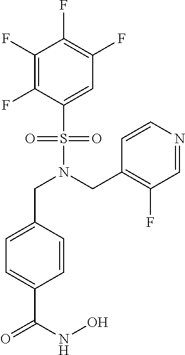 [SMILES: O=C(NO)c1ccc(CN(Cc2ccncc2F)S(=O)(=O)c2cc(F)c(F)c(F)c2F)cc1]

Compound I-28 was synthesized using General Procedure 1.

¹H NMR (400 MHz, Acetonitrile-d₃) δ 9.78 (s, 1H), 8.25 (s, 2H), 7.65 (dd, J=8.5, 2.9 Hz, 2H), 7.60 (t, J=8.3 Hz, 2H), 7.33-7.19 (m, 3H), 4.62-4.52 (m, 4H). ¹⁹F NMR (376 MHz, Acetonitrile-d₃) δ −133.47 (d, J=6.6 Hz), −134.76-−135.05 (m), −138.12 (ddt, J=29.6, 21.1, 10.1 Hz), −148.71 (dtt, J=46.8, 18.3, 8.3 Hz), −153.57 (q, J=19.1, 18.6 Hz).

### Synthesis of 3-fluoro-N-hydroxy-4-((2,3,4,5-tetrafluoro-N-(pyridin-3-ylmethyl)phenylsulfonamido)methyl)benzamide (Compound I-30)

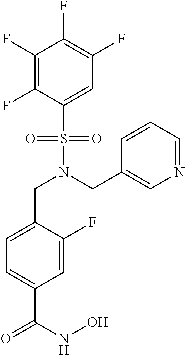 [SMILES: O=C(NO)c1ccc(CN(Cc2cccnc2)S(=O)(=O)c2cc(F)c(F)c(F)c2F)c(F)c1]

Compound I-30 was synthesized using General Procedure 1.

¹H NMR (400 MHz, Acetonitrile-d₃) δ 8.40 (s, 1H), 8.34 (s, 1H), 7.68 (dd, J=22.6, 7.2 Hz, 1H), 7.66-7.56 (m, 1H), 7.52-7.39 (m, 1H), 7.39-7.25 (m, 2H), 7.24 (dd, J=7.6, 4.7 Hz, 1H), 4.58 (s, 2H), 4.55 (s, 2H). ¹⁹F NMR (376 MHz, Acetonitrile-d₃) δ −117.37 (t, J=9.0 Hz), −118.16-−118.29 (m), −134.98 (ddt, J=21.1, 14.0, 7.1 Hz), −138.02-−138.24 (m), −148.64-−148.97 (m), −153.50-−153.73 (m). ¹³C NMR (101 MHz, Acetonitrile-d₃) δ 148.69, 148.32, 148.16, 137.13, 136.70, 132.00, 131.36, 123.85, 123.66, 122.76, 121.24, 117.33, 113.80, 112.93, 112.71, 50.24, 49.92, 46.16.

### Synthesis of N-hydroxy-4-((2,3,4,5-tetrafluoro-N-(pyridin-3-ylmethyl)phenylsulfonamido)methyl)-3-(trifluoromethyl)benzamide (Compound I-31)

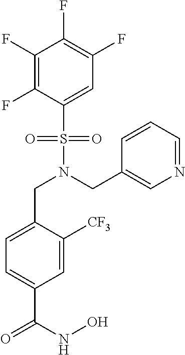 [SMILES: O=C(NO)c1ccc(CN(Cc2cccnc2)S(=O)(=O)c2cc(F)c(F)c(F)c2F)c(C(F)(F)F)c1]

Compound I-31 was synthesized using General Procedure 1.

¹H NMR (400 MHz, Acetonitrile-d₃) δ 8.32-8.28 (m, 1H), 8.25 (s, 1H), 7.93 (s, 1H), 7.86 (d, J=12.6 Hz, 2H), 7.80 (d, J=8.2 Hz, 1H), 7.65 (dd, J=34.3, 8.0 Hz, 2H), 7.52 (d, J=7.8 Hz, 1H), 7.24-7.14 (m, 1H), 4.75 (d, J=6.8 Hz, 2H), 4.54 (s, 2H). ¹⁹F NMR (376 MHz, Acetonitrile-d₃) δ −60.07 (d, J=32.6 Hz), −134.36 (dtt, J=22.1, 15.9, 7.6 Hz), −137.81 (dtd, J=27.3, 17.8, 15.5, 8.1 Hz), −148.38 (dtt, J=34.5, 17.9, 8.1 Hz), −153.11-−153.36 (m).

### Synthesis of 3-fluoro-N-hydroxy-4-((2,3,4,5-tetrafluoro-N-((2-fluoropyridin-3-yl)methyl)phenylsulfonamido)methyl)benzamide (Compound I-32)

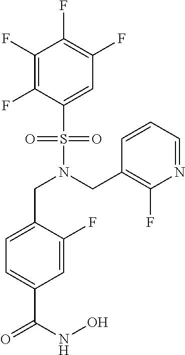 [SMILES: O=C(NO)c1ccc(CN(Cc2cccnc2F)S(=O)(=O)c2cc(F)c(F)c(F)c2F)c(F)c1]

Compound I-32 was synthesized using General Procedure 1.

¹H NMR (400 MHz, Acetonitrile-d₃) δ 9.71 (s, 1H), 8.10-7.98 (m, 1H), 7.77 (ddd, J=9.6, 7.4, 1.7 Hz, 1H), 7.65-7.54 (m, 1H), 7.52-7.36 (m, 2H), 7.34-7.26 (m, 1H), 7.15 (ddd, J=7.1, 5.0, 1.8 Hz, 1H), 4.62 (s, 2H), 4.57 (s, 2H). ¹⁹F NMR (376 MHz, Acetonitrile-d₃) δ −73.46 (dd, J=26.1, 10.0 Hz), −117.66-−117.78 (m), −118.66-−118.79 (m), −134.94 (dtt, J=23.1, 15.9, 7.3 Hz), −138.22 (dddd, J=29.7, 22.8, 11.3, 4.1 Hz), −148.75 (ddd, J=27.5, 18.5, 8.2 Hz), −148.96 (td, J=19.3, 9.3 Hz), −153.56-−153.85 (m).

### Synthesis of N-cyclopropyl-2,3,4,5,6-pentafluoro-N-(4-(hydroxycarbamoyl)benzyl)benzamide (Compound I-37)

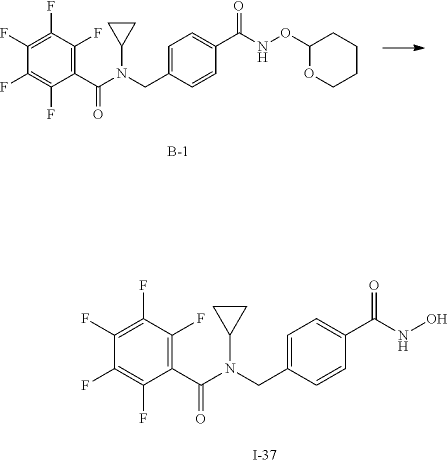

General Procedure 2.

The hydroxamate ester precursor B-1 (170 mg, 0.351 mmol) was dissolved in 4.0 M HCl (in 1,4-dioxan, 4 mL) and reacted for 4 hours in air at RT. After this time period, the reaction was concentrated and purified on preparative-HPLC to isolate the desired product as a white solid. This was further suspended in a 1:3 mixture of HPLC grade acetonitrile and Milli-Q water (4 mL) and lyophilized in vacuo at −50° C. overnight to isolate the final product as a white solid (120 mg, 88%).

¹H δ/ppm (400 MHz, Acetonitrile-d₃) 0.36-0.73 (m, 4H, 2CH₂), 2.63 (dt, J=7.0, 2.9 Hz, 1H, CH), 4.79 (s, 2H, CH₂), 7.40 (d, J=7.8 Hz, 2H, 2CH), 7.83 (d, J=7.9 Hz, 2H, 2CH), 8.09 (s, 1H, NH), ¹³C δ/ppm (101 MHz, Acetonitrile-d₃) 7.7, 29.9, 49.9, 125.7, 127.7, 128.3, 138.4, 141.8, 161.1. ¹⁹F δ/ppm (376 MHz, Acetonitrile-d₃) −163.3-to −161.8 (m, 2F, 2 CF), −155.4 (m, 1F, CF), −144.6-to −142.8 (m, 2F, 2 CF). HR-MS (ESI+) m/z calcd for [C₁₈H₁₄F₅N₂O₃]⁺: 400.30, found: 401.10. HPLC (I) t_{R}=13.85 min (98.0%); HPLC (II) t_{R}=19.39 min (99.6%).

### Synthesis of 4-((N-cyclopropyl-2,3,4,5,6-pentafluorophenylsulfonamido)methyl)-N-hydroxybenzamide (Compound I-4)

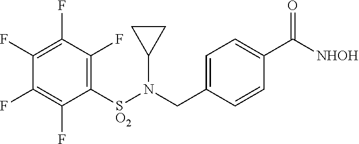

I-4 was synthesized in a similar manner to I-37.

1H NMR (400 MHz, Acetone-d6) δ 10.83 (bs, 1H), 7.87 (d, J=8.3 Hz, 2H), [7.52 (d, J=8.3 Hz, 2H rotamer #1), 7.46 (d, J=8.0 Hz, 2H rotamer #2)], [4.68 (s, 2H rotamer #1), 4.65 (s, 2H rotamer #2)], 2.59-2.49 (m, 1H), 0.86-0.51 (m, 4H). 13C NMR (101 MHz, Acetone-d6) δ 140.64, 131.74, 128.25, 127.17, 125.81, 30.27, 6.31. 19F NMR (376 MHz, Chloroform-d) δ −134.84-−135.26 (m, 2F), −145.36 (t, J=20.5 Hz, 1F), −157.84-−158.96 (m, 2F). HRMS (ESI+) m/z calcd for [C17H15F5N2O4S]+: 437.0589, found: 437.0588

### Synthesis of 4-((N-cyclopropyl-2,3,4,5-tetrafluorophenylsulfonamido)methyl)-N-hydroxybenzamide (Compound I-13)

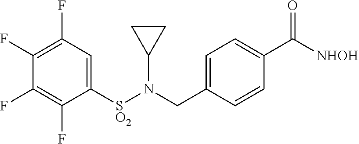

I-13 was synthesized in a similar manner to I-37.

¹H NMR (400 MHz, Acetone-d₆) δ 10.79 (bs, 1H), 8.18 (bs, 1H), [7.86 (d, J=8.3 Hz, 2H rotamer #1), 7.82 (d, J=8.2 Hz, 2H rotamer #2)]), 7.76 (dddd, J=9.6, 8.2, 5.8, 2.6 Hz, 1H), [7.52 (d, J=8.0 Hz, 2H rotamer #1), 7.40 (d, J=8.1 Hz, 2H rotamer #2)], [4.65 (s, 1H rotamer #1), 4.59 (s, 1H rotamer #2)], 2.52-2.43 (m, 4H), 0.71-0.62 (m, 1H). ¹³C NMR (101 MHz, DMSO-d₆) δ 139.19, 131.02, 127.05, 125.75, 111.72 (dd, J=21.9, 3.3 Hz), 52.54 (d, J=3.1 Hz), 28.95, 5.30. ¹⁹F NMR (376 MHz, Acetone-d₆) δ −134.84 (ddt, J=21.3, 14.0, 7.4 Hz, 1F), −138.26-−138.43 (m, 1F), −149.58 (tt, J=20.0, 8.2 Hz, 1F), −153.95 (ddt, J=21.9, 19.2, 3.4 Hz, 1F). HRMS (ESI+) m/z calcd for [C17H15F4N2O4S]+: 419.0683, found: 419.0681

### General Procedure for Compounds I-36, I-39, I-35, I-40, I-33 and I-34

To a nitrogen-purged solution of the hydroxamate ester (1.0 equiv.) in tetrahydrofuran (THF) and methanol (2:1, 0.05-0.1 M) was charged 10% Pd/C (0.04 equiv.) at room temperature (RT). The mixture was purged with hydrogen for 2-24 h before filtration through celite, washing with EtOAc, and concentrated in vacuo. Preparative high-performance liquid chromatography (HPLC), eluting at 20 mL/min, using gradient mixtures of (A) Milli-Q water with 0.1% (v/v) formic acid, and (B) HPLC-grade MeCN with 0.1% (v/v) formic acid, was used to isolate hydroxamic acids. Alternatively, preparative HPLC, eluting at 20 mL/min, using gradient mixtures of (A) Milli-Q water with 0.1% (v/v) trifluoroacetic acid (TFA), and (B) HPLC-grade MeCN, was used to isolate hydroxamic acids. Alternatively, preparative HPLC, eluting at 20 mL/min, using gradient mixtures of (A) Milli-Q water with 0.1% (v/v) formic acid, and (B) HPLC-grade MeCN, was used to isolate hydroxamic acids.

### Synthesis of 2,3,4,5,6-pentafluoro-N-(4-(hydroxycarbamoyl)benzyl)-N-isopropylbenzamide (Compound I-36)

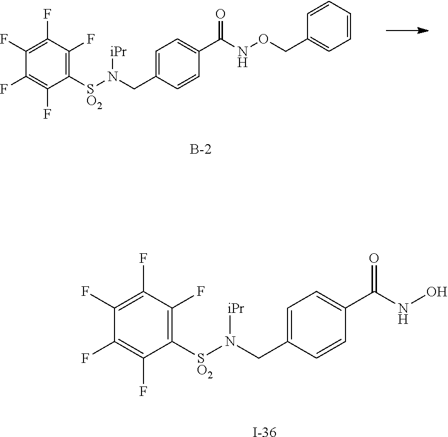

Using o-benzyl hydroxamate ester B-2 (453 mg, 0.920 mmol) and 10% Pd/C (40 mg, 0.037 mmol) in THF (7.0 mL) and methanol (3.5 mL), preparative HPLC eluted I-36 at 26.5-28.5 min, which was lyophilized to give an off-white solid (188 mg, 51%); 1H δ/ppm (400 MHz, DMSO-d6) [1.07 (d, J=6.6 Hz, 4.8H), 1.22 (d, J=6.8 Hz, 1.2H), 6H, 2CH3], [4.00 (p, J=6.4 Hz, 0.8H), 4.44-4.49 (m, 0.2H), 1H, CH], [4.51 (s, 0.4H), 4.74 (s, 1.6H), 2H, CH2), [7.21 (d, J=8.2 Hz, 0.4H), 7.37 (d, J=8.3 Hz, 1.6H), 2H, 2CH), [7.69 (d, J=8.3 Hz, 0.4H), 7.72 (d, J=8.3 Hz, 1.6H), 2H, 2CH], 9.01 (s, 1H, NH), 11.17 (s, 1H, OH); 13C δ/ppm (100 MHz, DMSO-d6) 19.7, 21.0, 43.4, 48.2, 51.0, 126.4, 126.7, 126.9, 131.3, 140.8, 141.6, 143.1, 144.9, 158.1, 164.0; 19F δ/ppm (54 MHz, DMSO-d6) [−160.5 (td, J=23.4, 6.9 Hz, 0.4F), −160.3 (tt, J=22.1, 5.2 Hz, 1.6F), 2F], −153.7 (t, J=22.0 Hz, 1F), [−143.2 to −143.3 (m, 1.6F), −142.5 to −142.6 (m, 0.4F), 2F]; LRMS (ESI−) m/z calcd for [C18H14F5N2O3]-: 401.09, found: 401.19; HRMS (ESI+) m/z calcd for [C18H16F5N2O3]+: 403.1072, found: 403.1076; HPLC (I) tR=19.24 min (96.0%); HPLC (II) tR=25.64 min (95.7%).

### Synthesis of 3-chloro-4-((N-cyclopropyl-2,3,4,5-tetrafluorophenylsulfonamido)methyl)-N-hydroxybenzamide (Compound I-39)

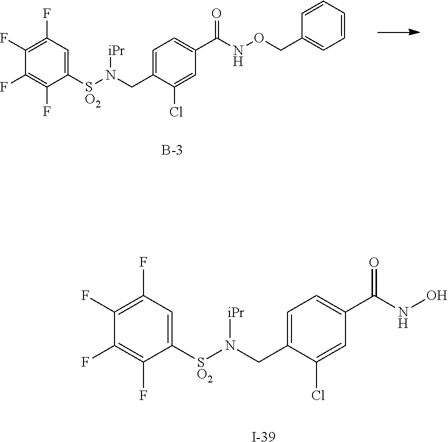

Using o-benzyl hydroxamate B-3 (40.0 mg, 0.074 mmol) and 10% Pd/C (3 mg, 0.003 mmol) in THF (1.0 mL) and methanol (0.5 mL), preparative HPLC eluted I-39 at 36.5-38.5 min, which was lyophilized to give an off-white solid (12.7 mg, 38%); 1H δ/ppm (400 MHz, DMSO-d6) 0.58-0.61 (m, 4H, 2CH2), CH signal obscured by the solvent, 4.61 (s, 2H, CH2), 7.53 (d, J=8.1 Hz, 1H, CH), 7.76 (dd, J=8.0, 1.5 Hz, 1H, CH), 7.81 (d, J=1.5 Hz, 1H, CH), 7.92-7.84 (m, 1H, CH), 9.17 (s, 1H, NH), 11.35 (s, 1H, OH); 13C δ/ppm (100 MHz, DMSO-d6) 6.6, 30.8, 51.4, 112.7, 113.0, 125.9, 127.7, 129.7, 132.2, 133.7, 137.5, 142.2, 143.4, 144.6, 148.7, 162.4; 19F δ/ppm (54 MHz, DMSO-d6) −151.70 (ddt, J=23.6, 20.7, 3.1 Hz, 1F), −147.15 (tt, J=21.6, 8.3 Hz, 1F), −136.69 (dddd, J=22.2, 12.9, 9.6, 3.6 Hz, 1F), −133.51 (ddt, J=21.0, 14.0, 7.7 Hz, 1F); LRMS (ESI−) m/z calcd for [C17H12ClF4N2O4S]—: 451.01, found: 451.17; HPLC (I) tR=16.58 min (99.9%); HPLC (II) tR=22.25 min (99.9%).

### Synthesis of 2,3,4,5,6-pentafluoro-N-(4-(hydroxycarbamoyl)benzyl)-N-methylbenzamide (Compound I-35)

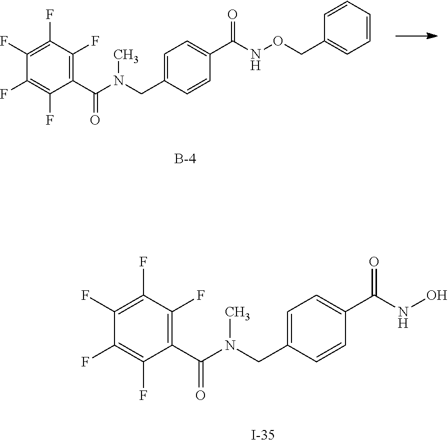

Using o-benzyl hydroxamate ester B-4 (445 mg, 0.958 mmol) and 10% Pd/C (41 mg, 0.038 mmol) in THF (10.0 mL) and methanol (5.0 mL), preparative HPLC eluted I-35 at 29.3-33.6 min, which was lyophilized to give an off-white solid (235 mg, 66%); ¹H δ/ppm (400 MHz, DMSO-d₆) [2.89 (s, 1.9H), 2.98 (s, 1.1H), 3H, CH₃), [4.57 (s, 0.7H), 4.77 (s, 1.3H), 2H, CH₂], [7.22 (d, J=8.2 Hz, 0.7H) 7.37 (d, J=8.2 Hz, 1.3H), 2H, 2CH], [7.74 (d, J=8.3 Hz) 7.77 (d, J=8.3 Hz) 2H, 2CH], 9.03 (s, 1H, NH), 11.20 (s, 1H, OH); ¹³C δ/ppm (100 MHz, DMSO-d₆) 32.9, 35.5, 49.7, 53.2, 127.0, 127.3, 127.3, 127.4, 132.1, 132.2, 136.2, 139.1, 139.4, 140.9, 143.4, 158.0, 158.2, 163.7, 163.8; ¹⁹F δ/ppm (54 MHz, DMSO-d₆) −160.5 to −160.4 (m, 2F), −153.5 to −153.3 (m, 1F), [−142.7 to −142.9 (m, 1.3F), −142.2 to −142.4 (m, 0.7F), 2F]; LRMS (ESI−) m/z calcd for [C₁₆H₁₀F₅N₂O₃]⁻: 373.06, found: 373.20; HPLC (I) t_{R}=14.87 min (97.0%); HPLC (II) t_{R}=18.47 min (97.0%). HRMS for [C₁₆H12F₅N₂O3] 375.0757, found: 375.0763

### Synthesis of N-hydroxy-4-((2,3,4,5,6-pentafluoro-N-(4-methoxybenzyl)phenylsulfonamido)methyl)benzamide (Compound I-40)

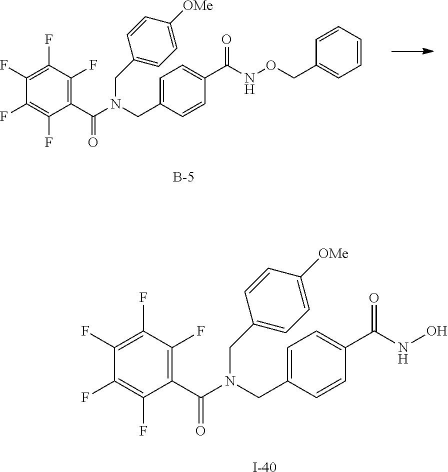

Using o-benzyl hydroxamate ester B-5 (90.0 mg, 0.148 mmol) and 10% Pd/C (6.32 mg, 0.00594 mmol) in THF (2.0 mL) and methanol (1.0 mL), preparative HPLC eluted I-40 at 21.3-23.5 min, which was lyophilized to give a white solid (57.3 mg, 74%); ¹H δ/ppm (400 MHz, CD₃CN) 3.74 (s, 3H, CH₃), 4.47 (s, 2H, CH₂), 4.60 (s, 2H, CH₂), 6.78 (d, J=8.7 Hz, 2H, 2CH), 7.10 (d, J=8.7 Hz, 2H, 2CH), 7.29 (d, J=8.1 Hz, 2H, 2CH), 7.64 (d, J=8.2 Hz, 2H, 2CH), 9.73 (br s, 1H, OH), NH not observed; ¹³C δ/ppm (100 MHz, CD₃CN) 51.8, 51.9, 54.9, 113.8, 126.8, 127.0, 128.3, 130.2, 131.1, 139.4, 139.8, 143.3, 159.5; ¹⁹F δ/ppm (54 MHz, CD₃CN) −161.5 to −161.3 (m, 2F), −149.2 (tt, J=6.6 and 20.2 Hz, 1F), −137.2 to −137.1 (m, 2F); LRMS (ESI+) m/z calcd for [C₂₂H₁₈F₅N₂O₅S]⁺: 517.09, found: 517.23; HRMS (ESI+) m/z calcd for [C₂₂H₁₈F₅N₂O₅S]⁺: 517.0858, found: 517.0851; HPLC (I) t_{R}=13.46 (98.7%); HPLC (II) t_{R}=19.15 (99.4%).

### Synthesis of 4-((N-cyclopropyl-2,3,4,5-tetrafluorophenylsulfonamido)methyl)-3-fluoro-N-hydroxybenzamide (Compound I-33)

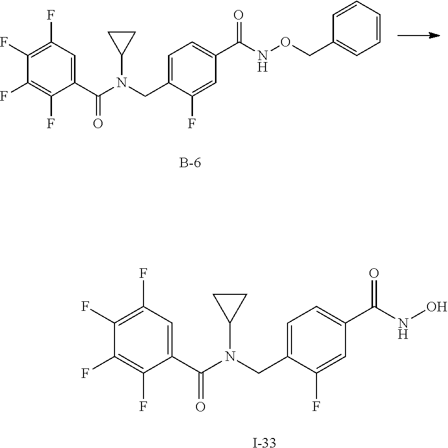

Using o-benzyl hydroxamate ester B-6 (120 mg, 0.194 mmol) and 10% Pd/C (8.25 mg, 0.00775 mmol) in THF (1.3 mL) and methanol (0.6 mL), preparative HPLC eluted I-33 at 35.3-35.9 min, which was lyophilized to give a white solid (52.2 mg, 62%); 1H δ/ppm (400 MHz, DMSO-d6) 0.56-0.65 (m, 4H, 4 CH), 2.40-2.45 (m, 1H, CH), 4.56 (s, 2H, CH2), 7.50 (t, J=7.9 Hz, 1H, CH), 7.54 (dd, J=1.4 and 11.0 Hz, 1H, CH), 7.62 (dd, J=1.4 and 8.0 Hz, 1H, CH), 7.80-7.86 (m, 1H, CH), 9.16 (s, 1H, NH), 11.32 (s, 1H, OH); 13C δ/ppm (100 MHz, DMSO-d6) 7.18, 31.0, 47.8, 113.11, 113.14, 113.3, 113.4, 114.1, 114.4, 123.3, 123.46, 123.48, 127.3, 127.4, 131.1, 131.2, 134.85, 134.92, 137.9, 140.0, 145.1, 147.6, 159.0, 161.5, 163.0; 19F δ/ppm (54 MHz, DMSO-d6) −151.9 to −151.7 (m, 1F), −147.2 (tt, J=8.3 and 22.0 Hz, 1F), −136.8 to −136.7 (m, 1F), −133.8 to −133.6 (m, 1F), −117.3 to −117.2 (m, 1F); LRMS (ESI+) m/z calcd for [C₁₇H₁₄F₅N2O4S]+: 437.06, found: 437.11; HRMS (ESI+) m/z calcd for [C17H14F5N2O4S]+: 437.0579, found: 437.0589; HPLC (I) tR=16.63 min (99.7%); HPLC (II) tR=21.90 min (99.8%).

### Synthesis of 2,3,4,5-tetrafluoro-N-(2-fluoro-4-(hydroxycarbamoyl)benzyl)benzamide (Compound I-34)

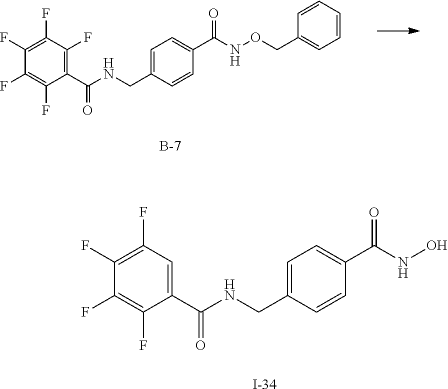

Using o-benzyl hydroxamate ester B-7 (120 mg, 0.194 mmol) and 10% Pd/C (8.25 mg, 0.00775 mmol) in THF (1.3 mL) and methanol (0.6 mL).

¹H δ/ppm (400 MHz, DMSO-d₆) 4.54 (d, J=5.9 Hz, 2H, CH₂), 7.38 (d, J=8.3 Hz, 2H, 2CH), 7.74 (d, J=8.3 Hz, 2H, 2CH), 9.02 (s, 1H, NH), 9.51 (t, J=5.9 Hz, 1H, NH), 11.19 (s, 1H, OH); 130 δ/ppm (100 MHz, DMSO-d₆) 42.5, 127.1, 127.1, 131.7, 135.8, 138.1, 141.4, 141.9, 144.2, 156.8, 164.0; ¹⁹F δ/ppm (54 MHz, DMSO-d₆) −161.1 to −161.3 (m, 2F), −152.9 (t, J=22.1 Hz, 1F), −142.0 to −142.2 (m, 2F); LRMS (ESI+) m/z calcd for [C₁₅H₉F₅N₂O₃Na]⁺: 383.04, found: 383.16; HRMS (ESI+) m/z calcd for [C₁₅H₁₀F₅N₂O₃]⁺: 361.0606, found: 361.0607; HPLC (I) t_{R}=10.48 min (98.7%); HPLC (II) t_{R}=13.92 min (96.5%).

### General Procedure of the Synthesis of Compounds I-12, I-14, I-3, I-2, I-1

The hydroxamate ester precursor (120 mg) dissolved in 4.0 M HCl (in 1,4-dioxan, 4 mL) and reacted for 4 hours in air at RT. After this time period, the reaction was concentrated and purified on preparative-HPLC to isolate the desired product as a white solid. This was lyophilized in vacuo at −50° C. overnight to isolate the final product as a white solid (50 mg, 41.7%).

### Synthesis of N-hydroxy-4-((2,3,4,5-tetrafluoro-N-isopropylphenylsulfonamido)methyl)benzamide (Compound I-12)

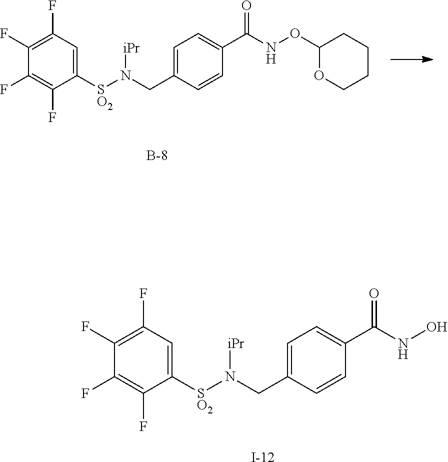

¹H NMR (400 MHz, Acetone-d₆) δ 10.82 (s, 1H), 7.84 (d, J=8.3 Hz, 2H), 7.72-7.59 (m, 1H), 7.54 (d, J=8.0 Hz, 1H), 7.48 (d, J=8.0 Hz, 1H), 4.64 (s, 1H) (rotamer #1), 4.61 (s, 1H) (rotamer #2), 4.28 (p, J=6.9 Hz, 1H), 1.09 (d, J=6.8 Hz, 6H). ¹⁹F NMR (376 MHz, Acetone-d₆) δ −135.93 (ddt, J=20.7, 13.3, 7.0 Hz, 1F), −138.04-−138.64 (m, 1F), −149.97 (tt, J=19.9, 8.2 Hz, 1F), −154.10 (ddd, J=21.6, 18.3, 3.2 Hz, 1F). HRMS (ESI+) m/z calcd for [C₁₇H₁₇F₄N₂O₄S]⁺: 421.08; found 421.0848

### Synthesis of 4-((N-cyclopropyl-2,3,5,6-tetrafluorophenylsulfonamido)methyl)-N-hydroxybenzamide (Compound I-14)

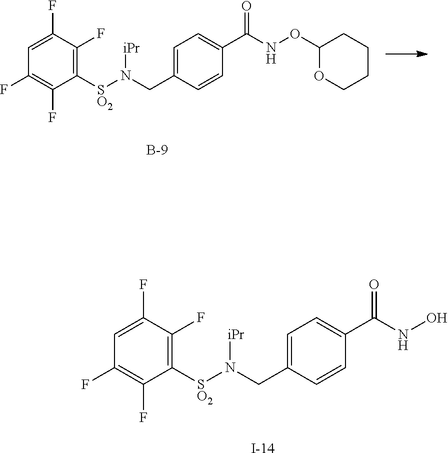

¹H NMR (400 MHz, Acetone-d₆) δ 10.81 (s, 1H), 8.02-7.79 (m, 3H), 7.53 (d, J=7.9 Hz, 1H), 7.46 (d, J=7.7 Hz, 1H), 4.70 (s, 1H) (rotamer #1), 4.67 (s, 1H) (rotamer #2), 2.56 (p, J=6.4, 3.5, 2.8 Hz, 1H), 0.80-0.61 (m, 4H). ¹⁹F NMR (376 MHz, Acetone-d₆) δ −137.15-−137.73 (m, 2F), −137.79-−138.20 (m, 2F).

### Synthesis of N-hydroxy-4-((2,3,4,5,6-pentafluoro-N-isopropylphenylsulfonamido)methyl)benzamide (Compound I-3)

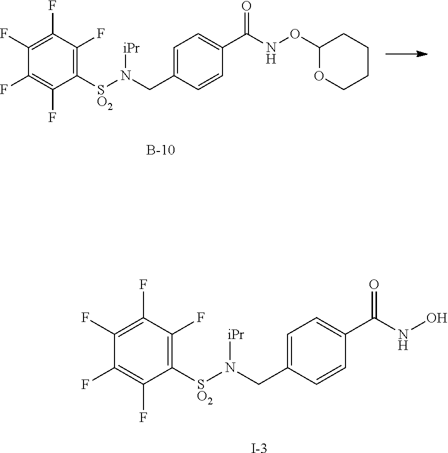

1H NMR (400 MHz, Acetonitrile-d₃) δ 9.76 (s, 1H), 7.71 (d, 2H), 7.51 (d, 2H), 4.60 (s, 2H), 1.11 (d, J=6.8 Hz, 6H). HRMS (ESI+) m/z calcd for [C₁₇H₁₆F₅N₂O4S]⁺: 439.07; found 439.0740.

### Synthesis of N-hydroxy-4-((2,3,4,5,6-pentafluoro-N-methylphenylsulfonamido)methyl)benzamide (Compound I-2)

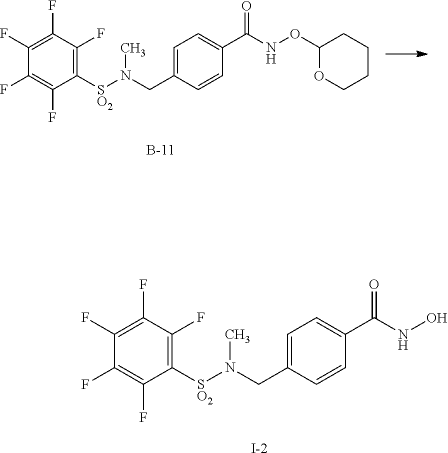

¹H NMR (400 MHz, Acetonitrile-d₃) δ 7.78 (d, J=8.3 Hz, 2H), 7.47 (d, J=8.3 Hz, 2H), 4.48 (s, 2H), 2.90-2.82 (m, 3H). 19F NMR (376 MHz, Chloroform-d) δ−134.19-−136.14 (m, 2F), −146.11 (tt, J=21.1, 6.7 Hz, 1F), −157.50-−159.50 (m, 2F). HRMS (ESI+) m/z calcd for [C₁₅H₁₂F₅N₂O₄S]⁺: 411.04; found 411.0425.

### Synthesis of N-hydroxy-4-((perfluorophenylsulfonamido)methyl)benzamide (Compound I-1)

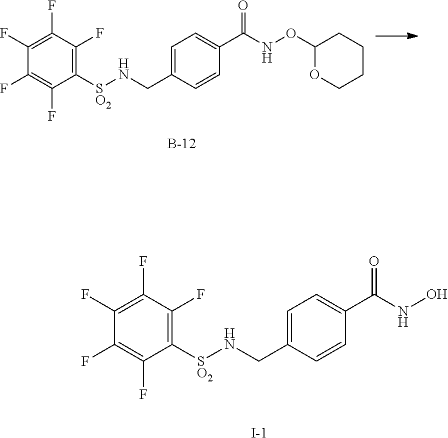

HRMS (ESI+) m/z calcd for [C₁₄H₁₀F₅N₂O₄S]⁺: 397.03; found 397.0269.

### Synthesis of 4-((3,5-dichloro-N-cyclopropyl-2,4,6-trifluorophenylsulfonamido)methyl)-N-hydroxybenzamide (Compound I-41)

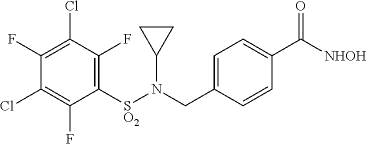

¹H NMR (400 MHz, Acetonitrile-d₃) δ 9.91 (bs, 1H), 7.75 (d, J=7.8 Hz, 2H), 7.48 (d, J=7.7 Hz, 2H), 4.63 (s, 2H), 2.49 (td, J=6.6, 3.5 Hz, 1H), 0.74-0.65 (m, 4H). ¹³C NMR (101 MHz, Acetonitrile-d₃) δ 140.90, 128.27, 127.15, 53.75, 30.40, 6.45. ¹⁹F NMR (376 MHz, Acetonitrile-d₃) δ −105.08 (t, J=6.9 Hz, 1F), −107.87 (d, J=6.9 Hz, 2F). LRMS (ESI+) m/z calcd for [C17H14C12F3N2O4S]+: 469.30, found: 469.00.

### Synthesis of 4-((3-chloro-N-cyclopropyl-2,4,6-trifluorophenylsulfonamido)methyl)-N-hydroxybenzamide (Compound I-42)

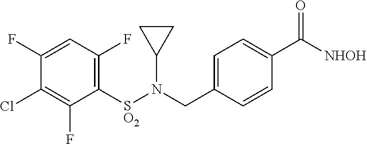

¹H NMR (400 MHz, Acetonitrile-d3) δ 7.75 (d, J=7.8 Hz, 2H), 7.48 (d, J=7.6 Hz, 2H), 7.23 (ddd, J=11.1, 9.2, 2.3 Hz, 1H), 4.61 (s, 2H), 2.59-2.40 (m, 1H), 0.77-0.56 (m, 4H). ¹³C NMR (101 MHz, Acetonitrile-d₃) δ 141.12, 131.23, 128.26, 127.15, 53.64, 30.29, 6.38. ¹⁹F NMR (376 MHz, Acetonitrile-d3) δ −104.04 (q, J=7.5 Hz, 1F), −105.46 (t, J=10.4 Hz, 1F), −105.71 (d, J=7.7 Hz, 1F). HRMS (ESI+) m/z calcd for [C17H15ClF3N2O4S]+: 435.0388, found: 435.0392.

### Synthesis of 3-fluoro-N-hydroxy-4-(((2,3,4,5-tetrafluoro-N-isopropylphenyl)sulfonamido)methyl)benzamide (Compound I-50)

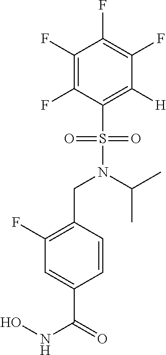 [SMILES: CC(C)N(Cc1ccc(C(=O)NO)cc1F)S(=O)(=O)c1cc(F)c(F)c(F)c1F]

¹H NMR (400 MHz, Acetone) δ 10.85 (s, 1H), 7.88-7.40 (m, 4H), 4.64 (s, 2H), 4.47-4.18 (m, 1H), 1.10 (dd, J=6.8, 3.0 Hz, 6H), ¹⁹F NMR (376 MHz, MeOD) δ −118.98-−119.12 (m, 1F), −136.34 (ddt, J=20.7, 13.7, 7.1 Hz, 1F), −138.56 (dt, J=21.3, 11.2 Hz, 1F), −150.04 (dq, J=19.5, 10.3, 9.2 Hz, 1F), −154.73 (d, J=19.7 Hz, 1F). HRMS (ESI+) m/z calcd for [C₁₇H₁₆F₅N₂O₄S]+: 439.0745, found: 439.0710. HPLC (I) t_{R}=34.42 min; HPLC (II) t_{R}=36.77 min (99%).

### Synthesis of 4-(((N-cyclopentyl-2,3,4,5-tetrafluorophenyl)sulfonamido)methyl)-N-hydroxybenzamide (I-51)

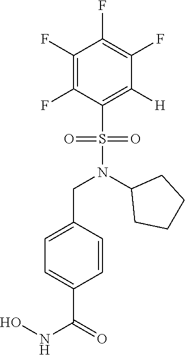 [SMILES: O=C(NO)c1ccc(CN(C2CCCC2)S(=O)(=O)c2cc(F)c(F)c(F)c2F)cc1]

¹H NMR (400 MHz, Acetone) δ 7.82 (d, J=8.3 Hz, 2H), 7.74-7.62 (m, 1H), 7.47 (dd, J=21.2, 8.1 Hz, 2H), 4.64 (d, J=9.6 Hz, 2H), 4.34 (t, J=8.3 Hz, 1H), 1.73-1.34 (m, 8H). ¹⁹F NMR (376 MHz, Acetone) δ −135.59 (ddp, J=20.3, 13.5, 6.9 Hz, 1F), −138.19-138.40 (m, 1F), −149.65-−150.03 (m, 1F), −154.05-−154.29 (m, 1F). HRMS (ESI+) m/z calcd for [C₁₉H₁₉F₄N₂O₄S]+: 447.0996, found: 447.1002. HPLC (I) t_{R}=34.77 min; HPLC (II) t_{R}=36.03 min (99%).

### Synthesis of 2,5-difluoro-N-hydroxy-4-(((2,3,4,5-tetrafluoro-N-isopropylphenyl)sulfonamido)methyl)benzamide (I-52)

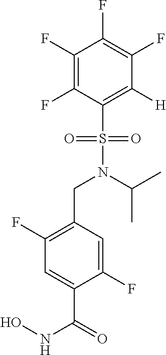 [SMILES: CC(C)N(Cc1cc(F)c(C(=O)NO)cc1F)S(=O)(=O)c1cc(F)c(F)c(F)c1F]

¹H NMR (500 MHz, cd₃cn) δ 9.69 (s, 1H), 7.65-7.55 (m, 1H), 7.49 (ddd, J=28.5, 10.3, 5.6 Hz, 1H), 7.30 (ddd, J=30.8, 10.9, 5.9 Hz, 1H), 4.53 (d, J=5.5 Hz, 2H), 4.37-4.00 (m, 1H), 1.05 (t, J=7.1 Hz, 6H). ¹⁹F NMR (376 MHz, CD₃CN) δ −117.53 (dd, J=19.6, 9.0 Hz, 1F), −123.69-−124.10 (m, 1F), −135.46 (ddt, J=21.4, 13.9, 6.7 Hz, 1F), −138.13-−138.32 (m, 1F), −149.03 (ddd, J=27.5, 18.7, 8.1 Hz, 1F), −153.36-−153.54 (m, 1F). HRMS (ESI+) m/z calcd for [C₁₇H₁₅F₆N₂O₄S]+: 457.0651, found: 457.0649. HPLC (I) t_{R}=38.09 min; HPLC (II) t_{R}=36.87 min (99%).

### Synthesis of N-hydroxy-4-(((2,3,4,5-tetrafluoro-N-isobutylphenyl)sulfonamido)methyl)benzamide (I-54)

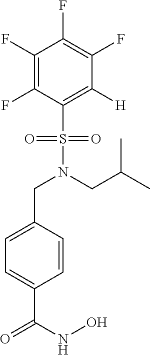 [SMILES: CC(C)CN(Cc1ccc(C(=O)NO)cc1)S(=O)(=O)c1cc(F)c(F)c(F)c1F]

¹H NMR (400 MHz, Acetone) δ 10.81 (s, 1H), 8.42 (s, 1H), 7.80 (dd, J=8.9, 2.4 Hz, 2H), 7.76-7.62 (m, 1H), 7.53-7.28 (m, 2H), 4.56 (d, J=10.1 Hz, 2H), 3.12 (h, J=6.6, 5.9 Hz, 2H), 1.72 (dp, J=13.7, 6.9, 6.4 Hz, 1H), 0.84-0.58 (m, 6H). ¹⁹F NMR (376 MHz, Acetone) δ −135.07 (ddt, J=20.8, 13.3, 6.9 Hz, 1F), −138.16-−138.34 (m, 1F), −149.83 (tt, J=19.4, 8.0 Hz, 1F), −154.05-−154.27 (m, 1F). HRMS (ESI+) m/z calcd for [C₈H₁₉F₄N₂O₄S]⁺: 435.0996, found: 435.0996. HPLC (I) t_{R}=29.04 min; HPLC (II) t_{R}=30.74 min (97.6%).

### Synthesis of N-hydroxy-4-(((2,3,4,5-tetrafluoro-N-(2-methoxyethyl)phenyl)sulfonamido)methyl)benzamide (I-55)

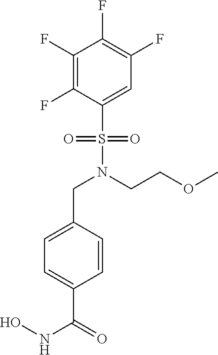 [SMILES: COCCN(Cc1ccc(C(=O)NO)cc1)S(=O)(=O)c1cc(F)c(F)c(F)c1F]

¹H NMR (400 MHz, Acetone) δ 10.70 (s, 1H), 7.82-7.67 (m, 2H), 7.57 (dddd, J=9.7, 8.2, 5.9, 2.5 Hz, 1H), 7.32 (d, J=8.0 Hz, 2H), 4.54 (s, 2H), 3.31 (t, J=5.4 Hz, 2H), 3.21 (t, J=5.2 Hz, 2H), 2.91 (s, 3H). Hydroxamic acid OH proton was not observed. ¹⁹F NMR (376 MHz, Acetone) δ −135.35 (ddt, J=20.6, 13.4, 6.9 Hz, 1F), −138.67-−138.91 (m, 1F), −150.31 (tt, J=20.3, 8.0 Hz, 1F), −154.76-−155.00 (m, 1F). ¹³C NMR (101 MHz, Acetone) δ 206.26, 141.15, 132.58, 129.13, 128.99, 128.09, 127.10, 113.49, 113.46, 70.22, 58.55, 51.93, 51.90, 47.87, 47.77. HRMS (ESI+) m/z calcd for [C₁₇H₁₇F₄N₂O₅S]⁺: 437.0789, found: 437.0842.

### Synthesis of N-hydroxy-3-methoxy-4-(((2,3,4,5-tetrafluoro-N-isopropylphenyl)sulfonamido)methyl)benzamide (I-53)

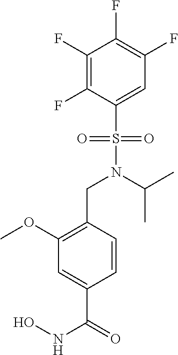 [SMILES: COc1cc(C(=O)NO)ccc1CN(C(C)C)S(=O)(=O)c1cc(F)c(F)c(F)c1F]

¹H NMR (400 MHz, Acetone) δ 10.89 (s, 1H), 7.70-7.22 (m, 4H), 4.56 (d, J=1.8 Hz, 2H), 4.41-4.22 (m, 1H), 3.92 (d, J=1.9 Hz, 3H), 1.11 (dd, J=6.8, 1.8 Hz, 6H). ¹⁹F NMR (376 MHz, Acetone) δ −135.97 (ddp, J=21.1, 14.5, 7.8, 7.3 Hz, 1F), −138.30-−139.27 (m, 1F), −149.93-−150.31 (m, 1F), −154.04-−155.43 (m, 1F). HRMS (ESI+) m/z calcd for [C₈H₁₉F₄N₂O₅S]⁺: 451.0945, found: 451.0971.

### Synthesis of (E)-3-(4-((N-cyclopropyl-2,3,4,5-tetrafluorophenylsulfonamido)methyl)phenyl)-N-hydroxyacrylamide (Compound I-64)

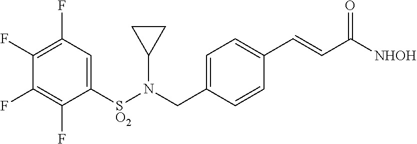

¹H NMR (400 MHz, Acetone-d₆) δ 7.73 (tdd, J=9.4, 6.9, 2.5 Hz, 1H), 7.59 (d, J=8.0 Hz, 2H), 7.41 (d, J=7.7 Hz, 2H), 7.17 (d, J=15.4 Hz, 1H), 6.70 (d, J=16.2 Hz, 1H), 4.59 (s, 2H), 2.67-2.35 (m, 1H), 0.82-0.53 (m, 4H). ¹³C NMR (101 MHz, Acetone-d₆) δ 137.01, 136.18, 132.24, 128.84, 127.79, 118.05, 112.76 (dd, J=22.1, 3.5 Hz), 53.72 (d, J=2.8 Hz), 30.26, 6.59. ¹⁹F NMR (376 MHz, Acetone-d₆) δ −134.83 (tt, J=13.8, 8.2 Hz), −138.33 (t, J=12.1 Hz), −149.42-−149.86 (m), −153.94 (q, J=19.4 Hz). HRMS (ESI+) m/z calcd for [C19H17F4N2O4S]+: 445.0840, found: 445.0841

### Synthesis of 2-(4-((N-cyclopropyl-2,3,4,5-tetrafluorophenylsulfonamido)methyl)phenyl)-N-hydroxyacetamide (Compound I-71)

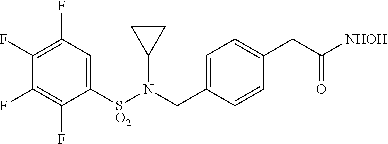

¹H NMR (400 MHz, Acetone-d₆) δ 10.33 (bs, 1H), 7.94-7.58 (m, 1H), 7.33 (d, J=9.4 Hz, 4H), [4.56 (s, 2H rotamer #1), 4.55 (s, 2H rotamer #2)], 3.47 (s, 2H), 2.42 (h, J=4.7, 4.1 Hz, 7H), 0.69-0.57 (m, 4H). ¹³C NMR (101 MHz, Acetone-d₆) δ 129.22, 128.36, 112.67 (d, J=21.7 Hz), 53.64, 33.05, 30.05, 6.55. ¹⁹F NMR (376 MHz, Acetone-d₆) δ −134.84-−135.05 (m), −138.41 (dtt, J=22.3, 9.2, 4.4 Hz), −149.63-−149.91 (m), −153.92-−154.10 (m). HRMS (ESI+) m/z calcd for [C18H17F4N2O4S]+: 433.0840, found: 433.0841

### Synthesis of (E)-N-hydroxy-3-(4-((2,3,4,5-tetrafluoro-N-methylphenylsulfonamido)methyl)phenyl)acrylamide (Compound I-62)

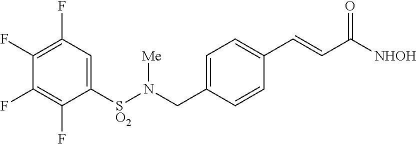

¹H NMR (400 MHz, Acetonitrile-d₃) δ 10.41 (bs, 1H), 7.84-7.71 (m, 1H), 7.60 (d, J=8.1 Hz, 2H), [7.42 (d, J=7.5 Hz, 1H rotamer #1), 7.37 (d, J=7.5 Hz, 1H rotamer #2)], 7.13 (d, J=14.8 Hz, 1H), 6.68 (d, J=16.0 Hz, 1H), [4.44 (s, 2H rotamer #1), 4.42 (s, 2H rotamer #2)], 2.83 (s, 3H). ¹³C NMR (101 MHz, Acetone-d₆) δ 136.67, 128.69, 127.10, 122.95, 116.13, 126.07, 79.97, 53.17, 33.75, 27.38. ¹⁹F NMR (376 MHz, Acetone-d₆) δ −134.95-−135.16 (m, 1F), −138.33-−138.53 (m, 1F), −149.97-−150.20 (m, 1F), −154.01-−154.21 (m, 1F). HRMS (ESI+) m/z calcd for [C17H15F4N2O4S]+: 419.07, found: 419.42

### Synthesis of (E)-2,3,4,5,6-pentafluoro-N-(4-(3-(hydroxyamino)-3-oxoprop-1-en-1-yl)benzyl)benzamide (Compound I-81)

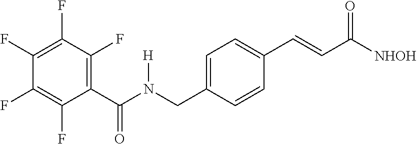

The hydroxamate ester (1.0 equiv) was treated with ice-cooled 4 M HCl in dioxane (final concentration 0.05-0.1 M) and stirred at RT for 18-24 h before concentrating in vacuo, azeotroping with CH₂Cl₂. Preparative high-performance liquid chromatography (HPLC), eluting at 20 mL/min, using gradient mixtures of (A) Milli-Q water with 0.1% (v/v) formic acid, and (B) HPLC-grade MeCN with 0.1% (v/v) formic acid, was used to isolate hydroxamic acids. Using o-THP hydroxamate ester (60.0 mg, 0.128 mmol) and 4M HCl/dioxane (2.6 mL) with the above procedure, preparative HPLC with gradient (A:B, 95:5→1:4, 50 min 410 min) eluted I-26 at 20.2-25.8 min, which was lyophilized to give a white solid (20.6 mg, 42%); ¹H δ/ppm (400 MHz, DMSO-d₆) 4.51 (d, J=5.9 Hz, 2H, CH₂), 6.45 (d, J=15.8 Hz, 1H, CH), 7.36 (d, J=8.0 Hz, 2H, 2CH), 7.44 (d, J=15.8 Hz, 1H, CH), 7.56 (d, J=7.9 Hz, 2H, 2CH), 9.04 (s, 1H, NH), 9.49 (t, J=5.8 Hz, 1H, NH), 10.75 (s, 1H, OH); ¹³C δ/ppm (100 MHz, DMSO-d₆) 42.6, 118.9, 127.6, 127.7, 133.8, 135.8, 137.9, 139.6, 141.8, 144.3, 156.7, 168.8; ¹⁹F δ/ppm (54 MHz, DMSO-d₆) −161.2 to −161.4 (m, 2F), −152.8 to −153.0 (m, 1F), −142.0 to −142.2 (m, 2F); LRMS (ESI−) m/z calcd for [C₁₇H₁₀F₅N₂O₃]—: 385.06, found: 385.18; HRMS (ESI+) m/z calcd for [C₁₇H₁₂F₅N₂O₃]⁺: 387.0756, found: 387.0763; HPLC (I) t_{R}=13.53 min (99.5%); HPLC (II) t_{R}=18.10 min (99.9%).

### Synthesis of (E)-N-hydroxy-3-(4-((2,3,4,5,6-pentafluoro-N-(4-methoxybenzyl)phenylsulfonamido)methyl)phenyl)acrylamide (Compound I-61)

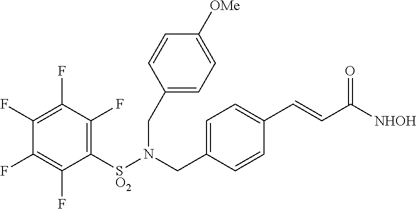

I-61 was synthesized using a similar procedure to I-26. Using o-THP hydroxamate ester (220 mg, 0.351 mmol) and 4 M HCl in dioxane (3.50 mL). Preparative HPLC, eluting at 20 mL/min, using gradient mixtures of (A) Milli-Q water with 0.1% (v/v) formic acid, and (B) HPLC-grade MeCN (A:B, 1:0→0:1, 50 min→10 min) was used to eluted I-6 at 23.8-25.0 min, which was lyophilized to give a white solid (38.5 mg, 20%); 1H δ/ppm (400 MHz, Acetone-d6) 3.75 (s, 3H, CH3), 4.52 (s, 2H, CH2), 4.61 (s, 2H, CH2), 6.56 (d, J=15.7 Hz, 1H, CH), 6.81 (d, J=8.7 Hz, 2H, 2CH), 7.16 (d, J=8.6 Hz, 2H, 2CH), 7.28 (d, J=8.0 Hz, 2H, 2CH), 7.51 (d, J=8.0 Hz, 2H, 2CH), 7.54 (d, J=15.7 Hz, 1H, CH), 8.43 (br s, 1H, NH), 10.35 (br s, 1H, OH); 13C δ/ppm (100 MHz, Acetone-d6) 51.5, 54.7, 113.8, 118.2, 126.9, 127.8, 128.9, 130.1, 131.7, 134.8, 137.3, 139.1, 145.8, 159.7; 19F δ/ppm (54 MHz, Acetone-d6) −161.9 to −161.8 (m, 2F), −149.9 (tt, J=6.5 and 20.7 Hz, 1F), −137.2 to −137.1 (m, 2F); LRMS (ESI+) m/z calcd for [C24H20F5N2O5S]+: 543.10, found: 543.23; HRMS (ESI+) m/z calcd for [C24H20F5N2O5S]+: 543.1009, found: 543.1008; HPLC (1) tR=13.83 and 13.96 min (44.4 and 55.3%); HPLC (II) tR=19.96 and 20.13 min (45.6 and 53.3%).

### Synthesis of (E)-N-hydroxy-3-(4-((2,3,4,5,6-pentafluoro-N-(4-fluorobenzyl)phenylsulfonamido)methyl)phenyl)acrylamide (Compound I-59)

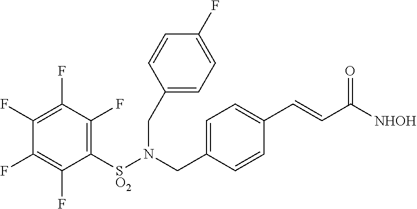

I-59 was synthesized using a similar procedure as I-26. Using o-THP hydroxamate ester (143 mg, 0.233 mmol) and 4 M HCl in dioxane (4.70 mL). Preparative HPLC, eluting at 20 mL/min, using gradient mixtures of (A) Milli-Q water with 0.1% (v/v) formic acid, and (B) HPLC-grade MeCN (A:B, 95:5→1:4, 60 min→10 min) was used to eluted I-4 at 38.5-40.0 min, which was lyophilized to give a white solid (53.1 mg, 42%); ¹H δ/ppm (400 MHz, DMSO-d₆) 4.55 (s, 4H, 2CH₂), 6.43 (d, J=15.8 Hz, 1H, CH), 7.10 (d, J=8.8 Hz, 2H, 2 CH), 7.21 (d, J=8.2 Hz, 2H, 2CH), 7.23-7.27 (m, 2H, 2CH), 7.40 (d, J=15.9 Hz, 1H, CH), 7.44 (d, J=8.6 Hz, 2H, 2CH), 9.05 (s, 1H, NH), 10.77 (s, 1H, OH); ¹³C δ/ppm (100 MHz, DMSO-d₆) 52.1, 52.2, 115.5, 115.7, 119.8, 127.8, 129.1, 129.3, 130.9, 131.0, 131.1, 132.0, 132.1, 134.8, 137.1, 138.1, 160.9, 163.1, 163.3; ¹⁹F δ/ppm (54 MHz, DMSO-d₆) −159.9 to −159.7 (m, 2F), −147.4 (tt, J=6.2 and 22.5 Hz, 1F), −136.4 to −136.3 (m, 2F), −114.6 to −114.5 (m, 1F); LRMS (ESI+) m/z calcd for [C₂₃H₁₆F₆N₂O₄SNa]⁺: 553.06, found: 553.25; HRMS (ESI−) m/z calcd for [C₂₃H₁₅F₆N₂O₄S]—: 529.0663, found: 529.0662; HPLC (I) t_{R}=15.58 min (97.6%); HPLC (II) t_{R}=22.18 min (97.3%).

### Synthesis of N-hydroxy-3-(4-((2,3,4,5,6-pentafluoro-N-(4-fluorobenzyl)phenylsulfonamido)methyl)phenyl)propanamide (Compound I-84)

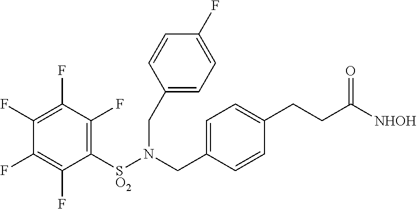

To a nitrogen-purged solution of the hydroxamate ester (1.0 equiv.) in tetrahydrofuran (THF) and methanol (2:1, 0.05-0.1 M) was charged 10% Pd/C (0.04 equiv.) at room temperature (RT). The mixture was purged with hydrogen for 2-24 h before filtration through celite, washing with EtOAc, and concentrated in vacuo. Preparative high-performance liquid chromatography (HPLC), eluting at 20 mL/min, using gradient mixtures of (A) Milli-Q water with 0.1% (v/v) formic acid, and (B) HPLC-grade MeCN with 0.1% (v/v) formic acid, was used to isolate the hydroxamic acid. Using o-benzyl hydroxamate ester (105 mg, 0.169 mmol) and 10% Pd/C (7.20 mg, 0.00677 mmol) in THF (1.2 mL) and methanol (0.6 mL). Preparative HPLC, eluting at 20 mL/min, using gradient mixtures of (A) Milli-Q water with 0.1% (v/v) formic acid, and (B) HPLC-grade MeCN, (A:B, 95:5→1:4, 60 min→10 min), was used to elute I-29 at 38.5-40.0 min, which was lyophilized to give a white solid (38.0 mg, 40%); ¹H δ/ppm (400 MHz, DMSO-d₆) 2.21 (t, J=7.9 Hz, 2H, CH₂), 2.76 (t, J=7.8 Hz, 2H, CH₂), 4.49 (s, 2H, CH₂), 4.52 (s, 2H, CH₂), 7.08-7.13 (m, 6H, 6 CH), 7.21-7.24 (m, 2H, 2CH), 8.72 (d, J=1.3 Hz, NH), 10.39 (s, 1H, OH); ¹³C δ/ppm (100 MHz, DMSO-d₆) 31.0, 34.3, 51.7, 52.2, 115.5, 115.7, 128.6, 128.9, 130.9, 130.95, 131.04, 132.10, 132.13, 133.2, 135.6, 136.5, 140.7, 141.3, 160.9, 163.3, 168.6; ¹⁹F δ/ppm (54 MHz, DMSO-d₆) −159.9 to −159.7 (m, 2F), −147.4 (tt, J=6.2 and 22.5 Hz, 1F), −136.5 to −136.4 (m, 2F), −114.7 to −114.6 (m, 1F); LRMS (ESI+) m/z calcd for [C₂₃H₁₈F₆N₂O₄SNa]⁺: 555.08, found: 555.26; HRMS (ESI+) m/z calcd for [C₂₃H₁₉F₆N₂O₄S]⁺: 533.0970, found: 533.0964; HPLC (I) t_{R}=15.10 min (95.5%); HPLC (II) t_{R}=21.88 min (93.8%).

### Synthesis of (E)-N-cyclopropyl-2,3,4,5,6-pentafluoro-N-(4-(3-(hydroxyamino)-3-oxoprop-1-en-1-yl)benzyl)benzamide (Compound I-83)

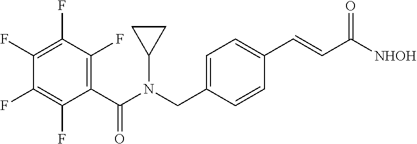

The hydroxamate ester (1.0 equiv) was treated with ice-cooled 4 M HCl in dioxane (final concentration 0.05-0.1 M) and stirred at RT for 18-24 h before concentrating in vacuo, azeotroping with CH2Cl2. Using o-THP hydroxamate ester (250 mg, 0.490 mmol) and 4 M HCl in dioxane (9.80 mL). Preparative HPLC, eluting at 20 mL/min, using gradient mixtures of (A) Milli-Q water with 0.1% (v/v) formic acid, and (B) HPLC-grade MeCN, (A:B, 9:1→1:4, 60 min→10 min) was used to isolate I-28 at 29.0-31.0 min, which was lyophilized to give a white solid (67.4 mg, 32%); ¹H δ/ppm (400 MHz, Acetone-d₆) 0.63-0.71 (m, 4H, 4CH), 2.63-2.68 (m, 1H, CH), 4.82 (s, 2H, CH₂), 6.60 (d, J=15.6 Hz, 1H, CH), 7.43 (d, J=8.0 Hz, 2H, 2CH), 7.59 (d, J=15.6 Hz, 1H, CH), 7.62 (d, J=8.0 Hz, 2H, 2CH), 8.42 (br s, 1H, NH), 10.30 (br s, 1H, OH); ¹³C δ/ppm (100 MHz, Acetone-d₆) 8.23, 30.2, 49.7, 113.3, 119.6, 127.6, 128.3, 128.4, 128.5, 128.6, 134.5, 136.5, 137.2, 138.3, 138.7, 141.1, 143.5, 161.0, 163.1; ¹⁹F δ/ppm (54 MHz, Acetone-d₆) −162.9 to −162.7 (m, 2F), −155.6 (t, J=20.0 Hz, 1F), −143.9 to −143.8 (m, 2F); LRMS (ESI+) m/z calcd for [C₂₀H₁₆F₅N₂O3]⁺: 427.11, found: 427.16; HRMS (ESI+) m/z calcd for [C₂₀H₁₆F₅N₂O₃]⁺: 427.1087, found: 427.1076; HPLC (I) t_{R}=14.73 min (99.1%); HPLC (II) t_{R}=20.12 min (99.4%).

### Synthesis of (E)-N-hydroxy-3-(4-((2,3,4,5,6-pentafluoro-N-(1-(4-fluorophenyl)ethyl)phenylsulfonamido)methyl)phenyl)acrylamide (Compound I-60)

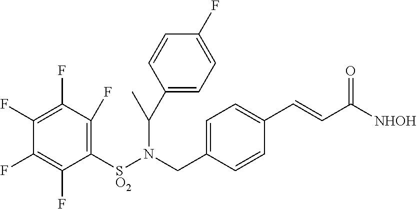

I-60 was synthesized in a similar manner to I-28. Using o-THP hydroxamate ester (111 mg, 0.177 mmol) and 4 M HCl in dioxane (3.60 mL). Preparative HPLC, eluting at 20 mL/min, using gradient mixtures of (A) Milli-Q water with 0.1% (v/v) formic acid, and (B) HPLC-grade MeCN (A:B, 95:5→0:1, 50 min→10 min), was used to isolate I-5 at 33.6-34.4 min, which was lyophilized to give a white solid (20.1 mg, 20%); 1H δ/ppm (400 MHz, DMSO-d6) 1.52 (d, J=7.1 Hz, 3H, CH3), 4.48 (d, J=16.2 Hz, 1H, CH), 4.59 (d, J=16.1 Hz, 1H, CH), 5.32 (q, J=7.1 Hz, 1H, CH), 6.42 (d, J=15.8 Hz, 1H, CH), 7.12 (t, J=8.8 Hz, 2H, 2 CH), 7.17 (d, J=8.1 Hz, 2H, 2CH), 7.35-7.38 (m, 3H, 3 CH), 7.41 (d, J=8.5 Hz, 2H, 2CH), 9.05 (s, 1H, NH), 10.77 (s, 1H, OH); 13C δ/ppm (100 MHz, DMSO-d6) 18.4, 48.4, 57.1, 115.4, 115.6, 117.1, 119.6, 127.6, 128.9, 130.2, 130.3, 134.4, 135.6, 138.2, 138.4, 160.9, 163.1, 163.3; 19F δ/ppm (54 MHz, Acetone-d6) −159.9 to −159.8 (m, 2F), −147.3 (tt, J=6.0 and 22.7 Hz, 1F), −136.4 to −136.2 (m, 2F), −114.33 to −114.26 (m, 1F); LRMS (ESI+) m/z calcd for [C24H19F6N2O4S]+: 545.10, found: 545.31; HRMS (ESI+) m/z calcd for [C24H19F6N2O4S]+: 545.0964, found: 545.0964; HPLC (1) tR=20.23 min (96.9%); HPLC (II) tR=30.94 min (97.1%).

### Synthesis of N-hydroxy-3-(6-((2,3,4,5,6-pentafluoro-N-(1-(4-fluorophenyl)ethyl)phenylsulfonamido)methyl)pyridin-3-yl)propanamide (Compound I-85)

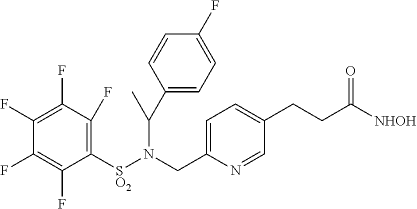

I-85 was synthesized in a similar manner to I-28. Using o-THP hydroxamate ester (49.0 mg, 0.0778 mmol) and 4 M HCl in dioxane (1.60 mL). Preparative high-performance liquid chromatography (HPLC), eluting at 20 mL/min, using gradient mixtures of (A) Milli-Q water with 0.1% (v/v) formic acid, and (B) HPLC-grade MeCN with 0.1% (v/v) formic acid (A:B, 95:5→35:65, 50 min 410 min), was used to isolate I-30 at 47.5-50.0 min, which was lyophilized to give a white solid (9.70 mg, 21%); ¹H δ/ppm (400 MHz, Acetone-d₆) 1.55 (d, J=7.1 Hz, 3H, 3 CH), 4.65 (d, J=17.0 Hz, 1H, CH), 4.72 (d, J=17.0 Hz, 1H, CH), 5.50 (q, J=7.0 Hz, 1H, CH), 6.62 (d, J=15.9 Hz, 1H, CH), 7.02 (t, J=8.8 Hz, 2H, 2CH), 7.25 (d, J=8.0 Hz, 1H, CH), 7.38-7.42 (m, 2H, 2CH), 7.53 (d, J=15.3 Hz, 1H, CH), 7.85 (d, J=7.5 Hz, 1H, CH), 8.50 (s, 1H, CH), 10.38 (br s, 1H, OH), NH not observed; ¹³C δ/ppm (100 MHz, Acetone-d₆) 17.4, 49.4, 56.8, 114.8, 115.0, 122.3, 129.7, 129.8, 132.1, 134.1, 135.58, 135.61, 148.4, 157.9, 161.0, 163.5, 168.6; ¹⁹F δ/ppm (54 MHz, Acetone-d₆) −162.2 to −162.1 (m, 2F), −149.6 (tt, J=6.3 and 20.7 Hz, 1F), −137.0 to −136.9 (m, 2F), −115.9 to −115.8 (m, 1F); LRMS (ESI+) m/z calcd for [C₂₃H₈F₆N₃O₄S]⁺: 546.09, found: 546.25; HRMS (ESI+) m/z calcd for [C₂₃H₁₈F₆N₃O₄S]⁺: 546.0905, found: 546.0917; HPLC (I) t_{R}=17.34 min (95.0%); HPLC (II) t_{R}=22.81 min (97.5%).

### Synthesis of N-hydroxy-3-(6-((2,3,4,5-tetrafluoro-N-(1-(3-fluorophenyl)ethyl)phenylsulfonamido)methyl)pyridin-3-yl)propanamide (Compound I-63)

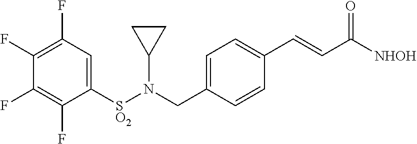

Compound I-63 was synthesized in a similar manner as I-28. Using o-THP hydroxamate ester (120 mg, 0.227 mmol) and 4 M HCl in dioxane (4.30 mL). Preparative high-performance liquid chromatography (HPLC), eluting at 20 mL/min, using gradient mixtures of (A) Milli-Q water with 0.1% (v/v) formic acid, and (B) HPLC-grade MeCN with 0.1% (v/v) formic acid (A:B, 1:0→1:1, 50 min 410 min), was used to isolate I-8 at 44.2-47.2 min, which was lyophilized to give an off-white solid (37.6 mg, 34%); ¹H δ/ppm (400 MHz, DMSO-d₆) 0.64 (m, 4H, 4CH), 2.58 (m, 1H, CH), 4.61 (s, 2H, CH₂), 6.55 (d, J=15.8 Hz, 1H, CH), 7.42-7.43 (m, 1H, CH), 7.48 (d, J=15.8 Hz, 1H, CH), 7.73-7.80 (m, 1H, CH), 7.98-8.00 (m, 1H, CH), 8.60 (s, 1H, CH), 9.11 (s, 1H, NH), 10.84 (s, 1H, OH); ¹³C δ/ppm (100 MHz, DMSO-d₆) 7.56, 31.1, 55.3, 113.0, 113.2, 115.7, 121.5, 122.9, 124.1, 131.9, 132.1, 135.0, 135.1, 149.0, 157.7, 162.7, 169.0, 171.3; ¹⁹F δ/ppm (54 MHz, DMSO-d₆) −152.3 (t, J=22.2 Hz, 1F), −147.7 to −147.5 (m, 1F), −137.2 to −137.1 (m, 1F), −133.6 to −133.4 (m, 1F); LRMS (ESI+) m/z calcd for [C₁₈H₁₆F₄N₃O₄S]⁺: 446.08, found: 446.19; HRMS (ESI+) m/z calcd for [C₁₈H₁₆F₄N₃O₄S]⁺: 446.0788, found: 446.0792; HPLC (I) t_{R}=14.18 min (97.6%); HPLC (II) t_{R}=19.45 min (99.9%).

### Synthesis of (E)-N-hydroxy-3-(4-((2,3,4,5-tetrafluoro-N-phenylphenylsulfonamido)methyl)phenyl)acrylamide (Compound I-66)

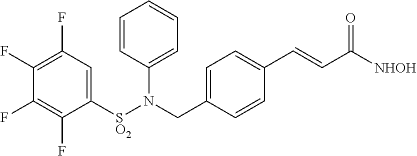

The hydroxamate ester precursor (120 mg) dissolved in 4.0 M HCl (in 1,4-dioxan, 4 mL) and reacted for 4 hours in air at RT. After this time period, the reaction was concentrated and purified on preparative-HPLC to isolate the desired product as a white solid. This was lyophilized in vacuo at −50° C. overnight to isolate the final product. ¹H NMR (400 MHz, Acetone-d₆) δ 7.58-6.96 (m, 11H), 6.57 (dd, J=22.8, 15.7 Hz, 1H), 5.05 (s, 1H) (rotamer #1), 5.01 (s, 1H) (rotamer #2). ¹⁹F NMR (376 MHz, Acetone-d₆) δ −133.20-−135.18 (m, 1F), −137.55-−139.44 (m, 1F), −148.47-−150.18 (m, 1F), −153.72 (q, J=22.2 Hz, 1F). HRMS (ESI+) m/z calcd for [C₂₂H₁₇F₄N₂O₄S]⁺: 481.08; found 481.08.

### Synthesis of (E)-2,3,4,5-tetrafluoro-N-(4-(3-(hydroxyamino)-3-oxoprop-1-en-1-yl)benzyl)benzamide (Compound I-82)

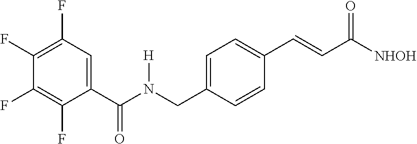

Compound I-27 was synthesized in a similar manner to I-26. ¹H NMR (400 MHz, Acetone-d₆) δ 8.25 (s, 1H), 7.75-7.15 (m, 7H), 6.75-6.47 (m, 1H), 4.66 (d, J=5.9 Hz, 2H). ¹⁹F NMR (376 MHz, Acetone-d₆) δ −140.12-−140.46 (m, 1F), −140.46-−140.66 (m, 1F), −153.14-−155.10 (m, 1F), −157.07 (ddd, J=21.7, 19.1, 2.6 Hz, 1F). HRMS (ESI+) m/z calcd for [C₁₇H₁₃F₄N₂O₃]⁺: 369.09; found 369.09.

### Synthesis of (E)-3-(4-((N-cyclopentyl-2,3,4,5-tetrafluorophenylsulfonamido)methyl)phenyl)-N-hydroxyacrylamide (Compound I-65)

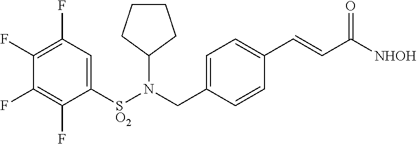

Compound I-65 was synthesized in a similar manner to compound I-26. ¹H NMR (400 MHz, Acetonitrile-d₃) δ 8.06 (s, 1H), 7.55 (m, 1H), 7.52 (d, J=7.9, 1.9 Hz, 2H), 7.41 (t, J=6.0 Hz, 1H), 7.34 (d, J=8.0 Hz, 2H), 7.27-7.03 (m, 2H), 4.52 (s, 2H), 4.33 (p, J=8.5 Hz, 1H), 1.82-1.20 (m, 8H). ¹⁹F NMR (376 MHz, Acetonitrile-d₃) δ −135.72 (dt, J=14.2, 6.6 Hz, 1F), −137.95-−139.82 (m, 1F), −149.83 (ddd, J=27.3, 18.4, 8.3 Hz, 1F), −154.02 (dd, J=21.9, 18.3 Hz, 1F). HRMS (ESI+) m/z calcd for [C₂₁H₂₁F₄N₂O₄S]⁺: 473.12; found 473.12.

### Synthesis of (E)-3-(4-((N-cyclopropyl-2,3,5,6-tetrafluorophenylsulfonamido)methyl)phenyl)-N-hydroxyacrylamide Compound (I-64)

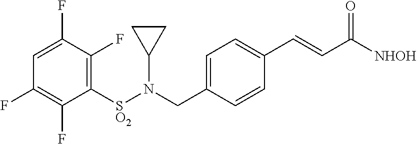

Compound I-9 was synthesized in a similar manner to compound I-26. ¹H NMR (400 MHz, Acetonitrile-d₃) δ 9.43 (s, 1H), 7.63 (d, J=10.1 Hz, 2H), 7.59 (d, J=8.0 Hz, 2H), 7.43 (d, J=8.0 Hz, 2H), 6.48 (d, J=13.2 Hz, 1H), 4.59 (s, 2H), 2.49 (p, 1H), 0.70 (s, 4H). ¹⁹F NMR (376 MHz, Acetonitrile-d₃) δ −137.71 (td, J=17.4, 15.5, 8.2 Hz, 2F), −137.89-−138.13 (m, 2F). HRMS (ESI+) m/z calcd for [C₁₉H₁₇F₄N₂O₄S]⁺: 445.08; found 445.0840.

### Synthesis of (E)-3-(4-((N-cyclopentyl-2,3,4,5,6-pentafluorophenylsulfonamido)methy)phenyl)-N-hydroxyacrylamide (Compound I-57)

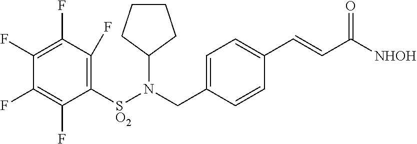

Compound I-57 was synthesized in a similar manner to I-26. ¹H NMR (400 MHz, Acetonitrile-d₃) δ 9.32 (s, 1H), 7.63-7.54 (m, 1H), 7.52 (d, J=8.1 Hz, 1H), 7.41 (d, J=8.0 Hz, 1H), 7.35 (d, J=8.1 Hz, 1H), 7.13 (d, J=16.1 Hz, 1H), 6.63 (d, J=16.1 Hz, 1H), 6.45 (d, J=16.2 Hz, 1H), 4.58 (s, 1H) (rotamer #1), 4.56 (s, 1H) (rotamer #2), 4.46-4.32 (m, 1H), 2.06-1.13 (m, 8H). ¹⁹F NMR (376 MHz, Chloroform-d) δ −135.22 (dt, J=20.9, 5.6 Hz, 2F), −146.11 (tt, J=21.1, 6.7 Hz, 1F), −158.01-−159.26 (m, 2F). HRMS (ESI+) m/z calcd for [C₂₁H₂₀F₅N₂O₄S]⁺: 491.11; found 491.1056.

### Synthesis of N-hydroxy-2-(4-((2,3,4,5-tetrafluoro-N-isopropylphenylsulfonamido)methyl)phenyl)acetamide (Compound I-70)

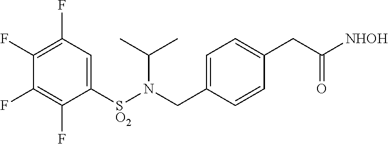

Compound I-70 was synthesized in a similar manner to I-26. HRMS (ESI+) m/z calcd for [C₁₈H₁₉F₄N₂O₄S]⁺: 435.10; found 435.0989.

### General Procedure for Preparation of Compounds of Formula B

Two drops of DMF and 2.5 equivalents of oxalyl chloride were added to 1 mmol of compound A. The mixture was stirred at room temperature for 1 hour before solvents were removed and dried under pressure. Solids obtained were redissolved in THF and added to a solution of 1.1 equivalent of tetrahydropyranyl O-hydroxylamine or 1.1 equivalent of O-benzylhydroxylamine and 1 equivalent of triethylamine. The mixture was stirred for 1 hour before diluting in water and extracting with ethyl acetate. The organic layer was washed with brine and dried over MgSO4. Flash chromatography was used to isolate the pure product.

C. Structure-Activity Relationship and In Vitro HDAC Inhibition and Selectivity

The exemplary compounds of Formula I and structurally related compounds were synthesized to assess their structure-activity relationship. The exemplary compounds were tested for potency against HDAC3, 6, 8 and 11 in an in vitro activity-based assay (see for example, Shouksmith, A. E. et al. J. Med Chem. 2019, 62(5), 2651-2665). The IC₅₀ values for each exemplary compound were used to calculate the selectivity of the exemplary compounds for Formula I to HDAC6. The selectivity of each exemplary compound for HDAC6 was determined from examining the potency of HDAC6 in relation to the highest binding affinity of the other HDAC isoforms tested.

Exemplary compounds I-1, I-4, I-8, I-11, I-15, I-29, I-31, and I-53 were found to be less than 5 times more selective towards HDAC6 compared the highest binding affinity of the other HDAC isoforms tested.

Exemplary compounds I-5, I-6, I-7, I-9, I-10, I-13, I-16, I-23 to I-28, I-38, I-39, I-41, I-42, I44, and I-51, were found to be between about 5 time to 30 times more selective towards HDAC6 compared the highest binding affinity of the other HDAC isoforms tested.

Exemplary compounds I-2, I-12, I-14, I-17, I-20, I-21, I-22, I-32 to I-37, I-43, I-46, I-49, I-50, I-52, I-54 and I-55 were found to be between about 30 time to 70 times more selective towards HDAC6 compared the highest binding affinity of the other HDAC isoforms tested.

Exemplary compound I-19, I-45 was found to be between about 70 time to 100 times more selective towards HDAC6 compared the highest binding affinity of the other HDAC isoforms tested.

Exemplary compounds I-3, I-18, I-30, and I-48 were found to be over 100 times more selective towards HDAC6 compared the highest binding affinity of the other HDAC isoforms tested.

In addition to testing against HDAC3, 6, 8 and 11, exemplary compounds I-13, I-18, I-34, and I-50 were tested against all HDAC isoforms which are shown in Table 1. These results demonstrated that exemplary compounds I-13, I-18, I-34 and I-50 have strong potency against HDAC6 with limited off-target effects against other human HDAC isoforms, suggesting that these exemplary compounds are not pan-HDAC inhibitors.

TABLE 1
IC₅₀ of exemplary compounds against
all human HDAC Isoforms (μM)

| HDAC Isoform | IC₅₀ (I-18) | IC₅₀ (I-13) | IC₅₀(I-34) | IC₅₀ (I-50) |
|---|---|---|---|---|
| HDAC1 | 0.908 | >1 | >1 | >1 |
| HDAC2 | >1 | >1 | >1 | >1 |
| HDAC3 | 0.373 | >1 | 0.215 | >1 |
| HDAC4 | >1 | >1 | >1 | >1 |
| HDAC5 | >1 | >1 | >1 | >1 |
| HDAC6 | 0.002 | 0.009 | >1 | 0.017 |
| HDAC7 | >1 | >1 | >1 | >1 |
| HDAC8 | 0.316 | 0.334 | 0.694 | >1 |
| HDAC9 | >1 | >1 | >1 | >1 |
| HDAC10 | 0.551 | >1 | >1 | >1 |
| HDAC11 | >1 | >1 | >1 | >1 |

Exemplary compounds I-57, I-58, I-59, I-60, I-63, I-64, I-66, I-67, I-68, I-74, I-81, I-82 and I-73 were found to have an IC₅₀ for HDAC6 of 0.010 μM-0.030 μM. Exemplary compounds I-56 and I-61 were found to have an IC₅₀ for HDAC6 of 0.030 μM-0.050 μM. Exemplary compound I-69 was found to have an IC₅₀ for HDAC6 of 0.050 μM-0.10 μM. Exemplary compounds I-70, I-71 and I-72 were found to have an IC₅₀ for HDAC6 of 0.100 μM-0.150 μM. Exemplary compounds I-65 and I-78, were found to have an IC₅₀ for HDAC6 of 0.150 μM-0.200 μM. Exemplary compounds I-73, I-75, I-76, I-77, I-79 and I-80 were found to have an IC₅₀ for HDAC6 of >0.200 μM.

D. Enzyme Kinetic Parameters

To determine the kinetic parameters for the formation and maintenance of the HDAC6:1-18 complex, several time-based experiments were performed, which are shown in Table 2. The exemplary compound I-18 showed a small, albeit noticeable change in the inhibition of the enzyme following a 3 h pre-incubation time period. Most notably, the residence time of I-18 was substantially longer than ricolinostat.

TABLE 2
Kinetic parameters of HDAC6:I-18 interaction.

| Parameter | I-18 | Riclinostat | Citarinostat |
|---|---|---|---|
| IC₅₀ (No Preincubation) | 4.0 nM | 6.7 nM | 6.1 nM |
| IC₅₀ (3 h Preincubation) | 2.0 nM | 5.3 nM | 4.0 nM |
| Residence Time | 69 min | 32 min | |
| kₒₙ | 9.6 × 10⁻⁵ nM⁻¹s⁻¹ | | |
| Kᵢ | 1.4 nM | | |

E. Kinetic Solubility

The kinetic solubility in PBS with 5% (v/v) DMSO for select compounds of the application was assessed quantitatively through analytical HPLC. The initial concentration of each exemplary compound was 300 μM. The exemplary compounds were compared to the benchmark compound Ricolinostat and all showed substantially increased solubility. Table 3 shows the results of kinetic solubility experiments.

TABLE 3
Kinetic Solubility of Select
HDAC6 Inhibitors

| | Saturation Concentration (μM) |
|---|---|
| Compounds | PBS + 5% DMSO |
| Ricolinostat | 26.1 ± 11.3 |
| I-18 | 142.4 ± 2.4 |
| I-30 | 46.0 ± 4.4 |
| I-12 | 74.5 ± 3.0 |
| I-13 | 88.1 ± 1.6 |

F. Efficacy Studies

The cytotoxicity of exemplary compounds of the application was tested in multiple cell lines including MV-4-11, MOLM-13, MCF-7, MRC9, K562 and MM.1S. The therapeutic index was evaluated by determining the toxicity of the exemplary compounds in healthy cell lines including fibroblasts and HUVEC. The data is shown in Tables 4 and 5.

TABLE 4
IC₅₀ values (μM) of HDAC targeting compounds in different cell lines

| | | | | | | | Normal | |
|---|---|---|---|---|---|---|---|---|
| | | | | | | | Human | |
| | | | | | | | Fibro | |
| Comp | MV-411 | MOLM-13 | MCF-7 | MRC9 | K562 | MM.1S | blast | HUVEC |
| Riclolinostat | 0.656 | 6.49 | | | 12.84 | | 5.7 | 4.9 |
| 1-1 | 11.07 | | | | | | | |
| 1-2 | 1.04 | | | | | | | |
| 1-7 | 0.3 | | | | | | | |
| 1-9 | | 5.2 | | | | | | |
| 1-10 | | 9.1 | | | | | | |
| 1-11 | | 3.5 | | | | | | |
| 1-13 | 0.465 | 2.0 | | >50 | 7.4 | | | |
| 1-14 | 5.7 | | | | | | | |
| 1-15 | 2.6 | | | | | | | |
| 1-16 | 0.19 | | | | | | | |
| 1-19 | | | | | 5.6 | | | |
| 1-20 | 0.48 | | | | | | | |
| 1-21 | 0.478 | 1.7 | | | 7.9 | | | |
| 1-22 | | 3 | | | 6.2 | | | |
| 1-24 | 2.02 | 9 | | | | | | |
| 1-25 | 0.23 | | | | | | | |
| 1-26 | 1.87 | | | | | | | |
| 1-27 | 0.843 | | | | | | | |
| 1-28 | 0.596 | 0.663 | | | | | | |
| 1-29 | 4.2 | 5.6 | 20.9 | | | | | |
| 1-30 | 0.607 | 2.9 | | | 9.3 | | 6.1 | 4.1 |
| 1-31 | 3.4 | 1.6 | 5 | | | | | |
| 1-32 | 0.389 | 1.98 | | | 8.6 | | 7.1 | 5.1 |
| 1-33 | 0.28 | | | | | | | |
| 1-34 | | | | >50 | | | | |
| 1-38 | 4.2 | 233.9 | | 13.8 | | | | |
| 1-42 | 0.741 | | | | | | | |
| 1-43 | 4.2 | 19.5 | | | 22.95 | | | |
| 1-44 | 4.1 | 5.6 | | | | | | |
| 1-45 | 1.4 | 4.9 | | | | 1.1 | | |
| 1-46 | 1.7 | | | | | 0.4 | | |
| 1-47 | 0.2 | 11.4 | | | | | | |
| 1-48 | 0.8 | 3.0 | | | | | | |
| 1-49 | 2.0 | 7.5 | | | | | | |
| 1-50 | 1.6 | | | >50 | | 1.2 | | |
| 1-51 | 2.8 | | | | | 2.7 | | |
| 1-52 | 1.0 | | | | | 0.4 | | |
| 1-54 | 0.5 | | | | | 0.7 | | |

TABLE 5
IC₅₀ values (μM) of HDAC targeting
compounds in different cell lines

| Compound | MV-411 | MOLM-13 | MRC9 | K562 |
|---|---|---|---|---|
| Riclolinostat | 0.656 | 6.49 | | 12.84 |
| I-56 | | | >50 | |
| I-57 | | | >50 | |
| I-59 | 0.59 | | | |
| I-60 | 0.47 | | | |
| I-65 | 2.43 | | | |
| I-65 | 1.94 | | | |
| I-66 | 0.84 | | | |
| I-67 | 1.14 | | | |
| I-68 | 0.80 | | | |
| I-69 | 1.6 | 1.8 | | |
| I-70 | 2.5 | | | |
| I-72 | 0.57 | | | |
| I-73 | 1.09 | | | |
| I76 | 0.589 | 1.2 | | |
| I-77 | 1.1 | 3.3 | | 72.8 |
| I-78 | 1.3 | 2.9 | | |
| I-79 | 0.329 | 7.9 | | 25.9 |
| I-80 | 0.338 | 7.6 | | 16.3 |
| I-82 | 0.59 | | | |
| I-83 | 0.75 | | | |

G. Potency of HDAC6 Inhibitors in Cellulo

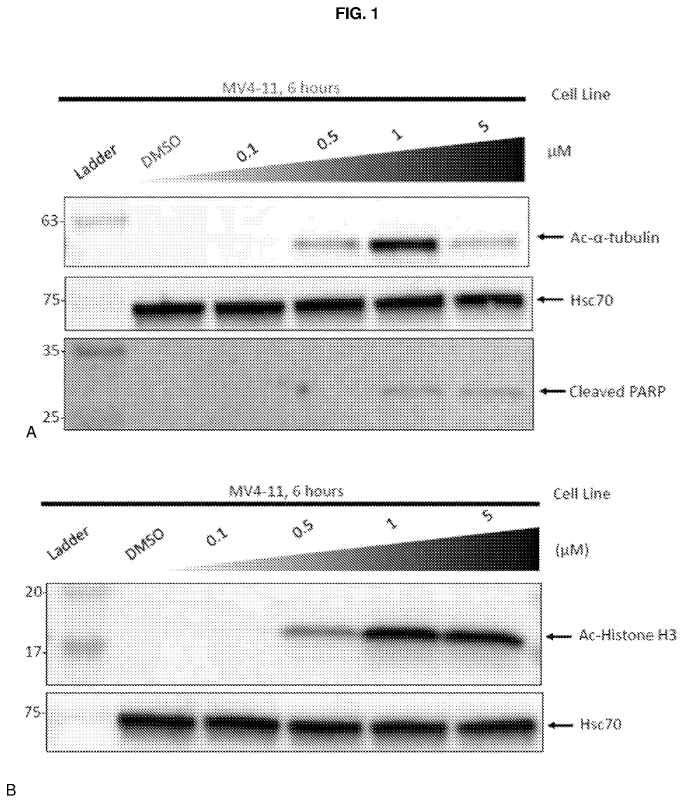

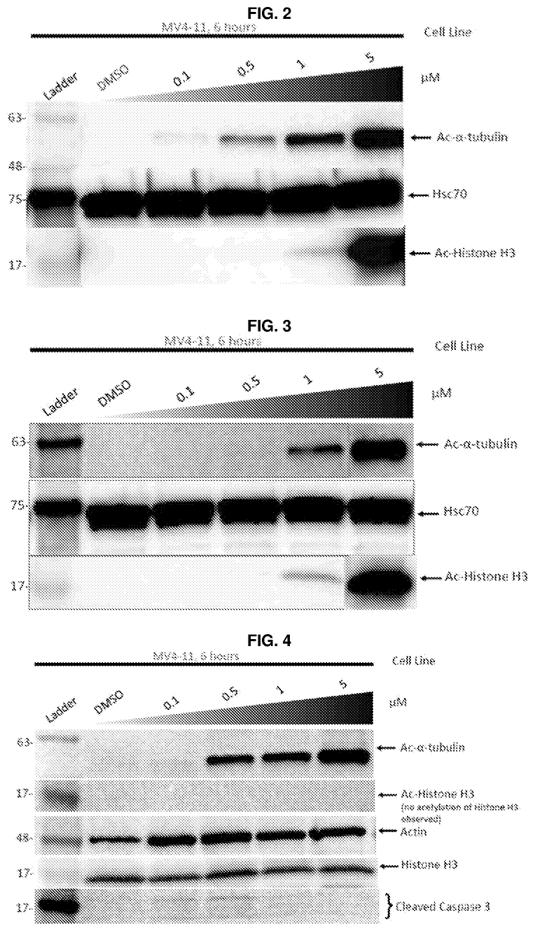

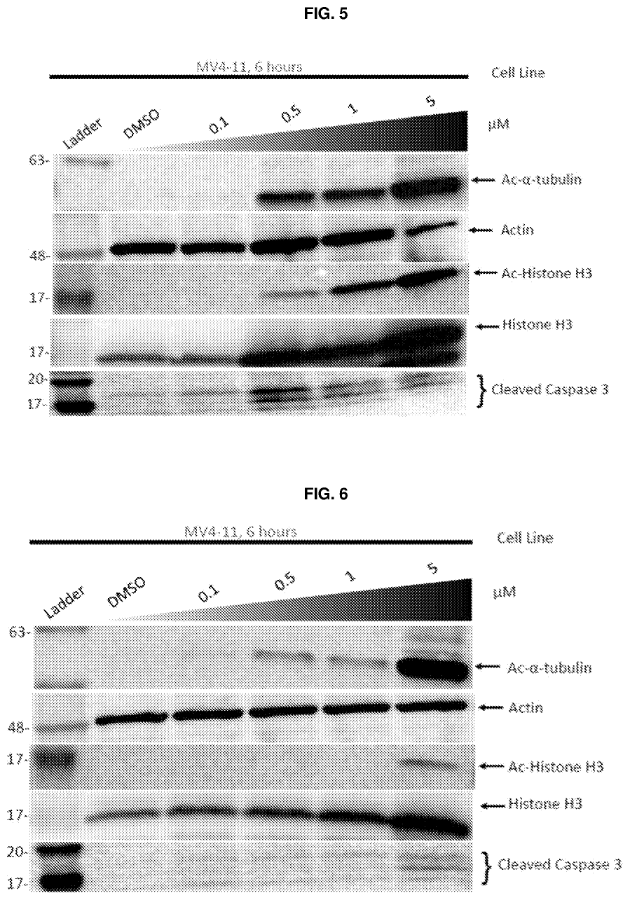

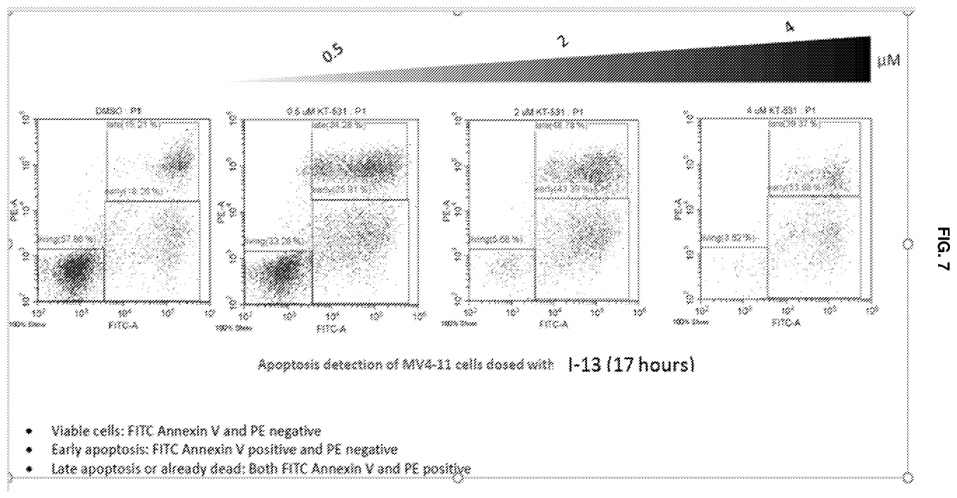

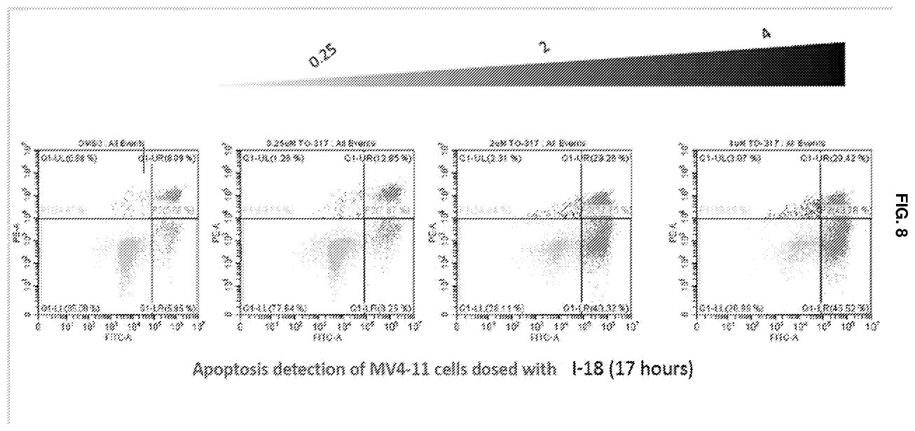

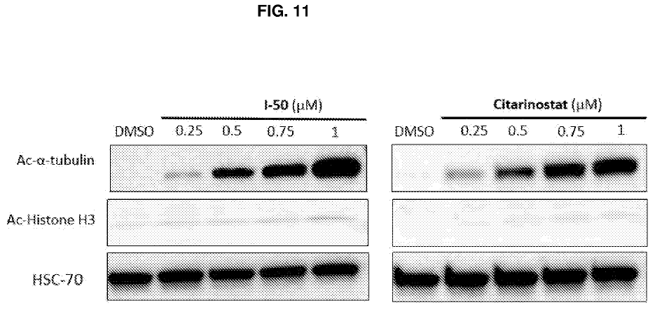

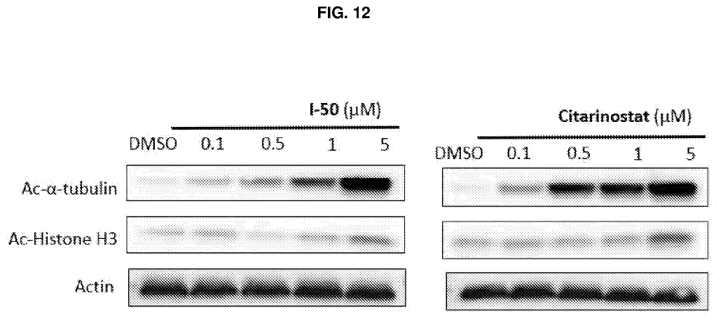

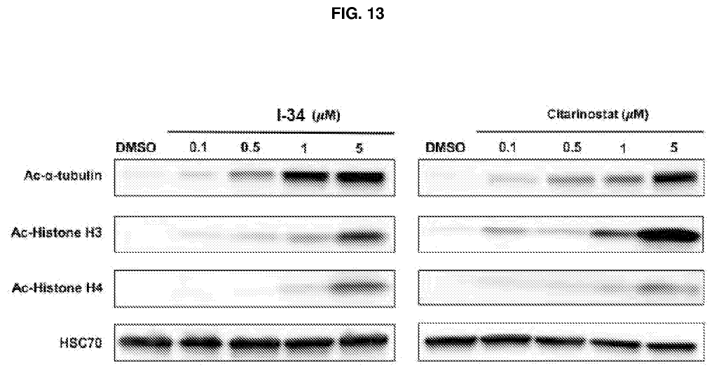

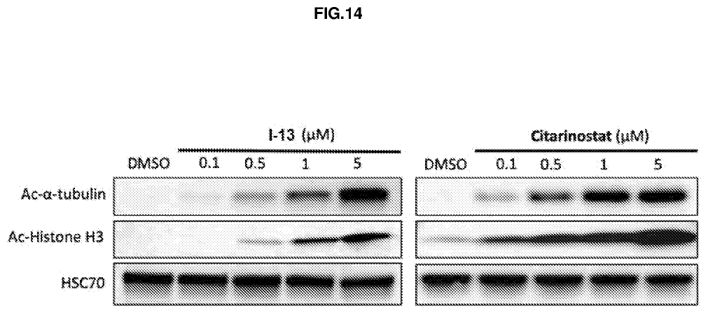

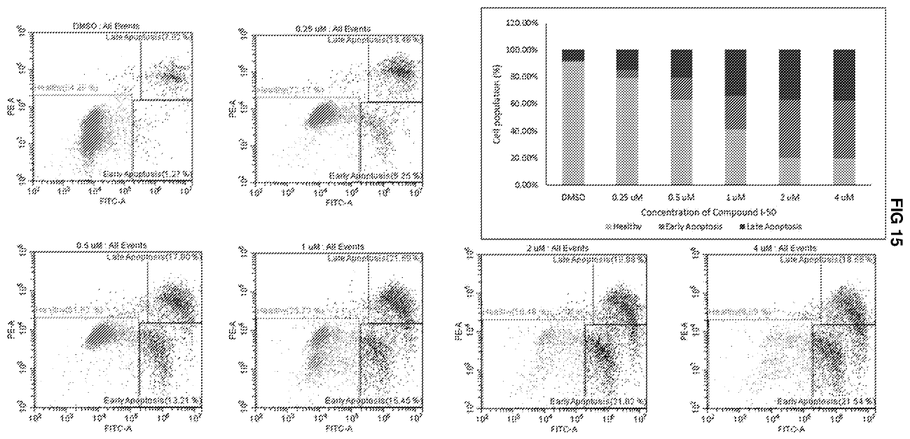

The mechanism of action for HDAC6 inhibitors in cellulo was interrogated through western blot analysis of MV-4-11 cells post 6 h treatment of increasing concentrations of either ricolinostat, I-4, I-3, I-18, I-13, and I-12. HDAC6 activity was probed through examining the presence of acetylated downstream targets of HDAC6, α-tubulin or histone H3. Increases in substrate acetylation at higher concentrations of drug suggest inhibition of HDAC6. FIGS. 1 to 6 shows the results from the Western blot analysis of cells treated with I-4, I-18, I-3, ricolinostat, I-13, or I-12 respectively. Similar Western blot analysis is shown for I-50 and citarinostat in MV-4-11 cells (FIG. 11) and MM.1S cells (FIG. 12), as well as for I-34 (FIG. 13) and I-13 (FIG. 14) in MV-4-11 cells.

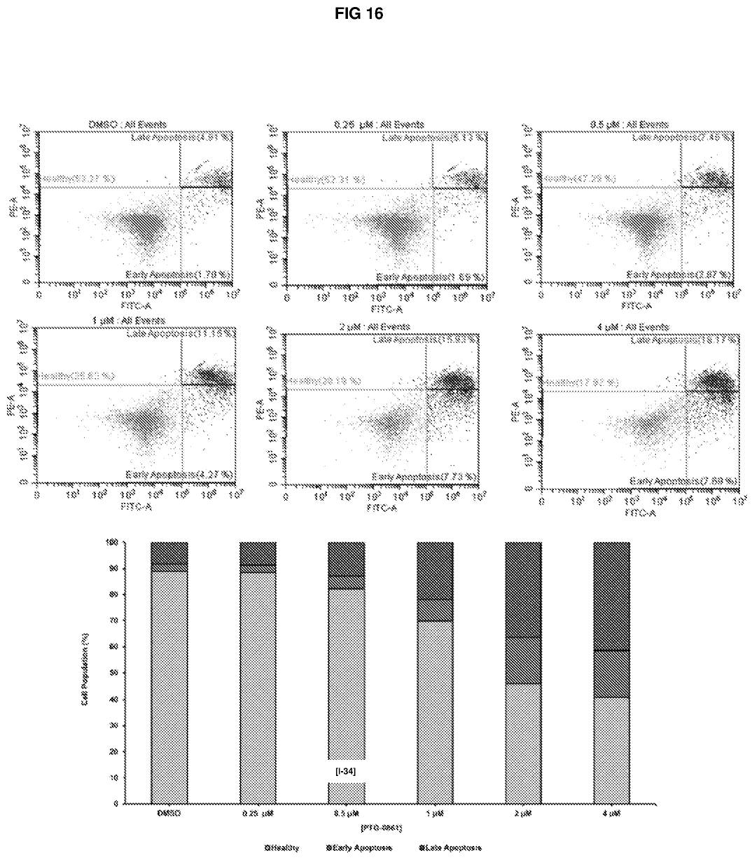

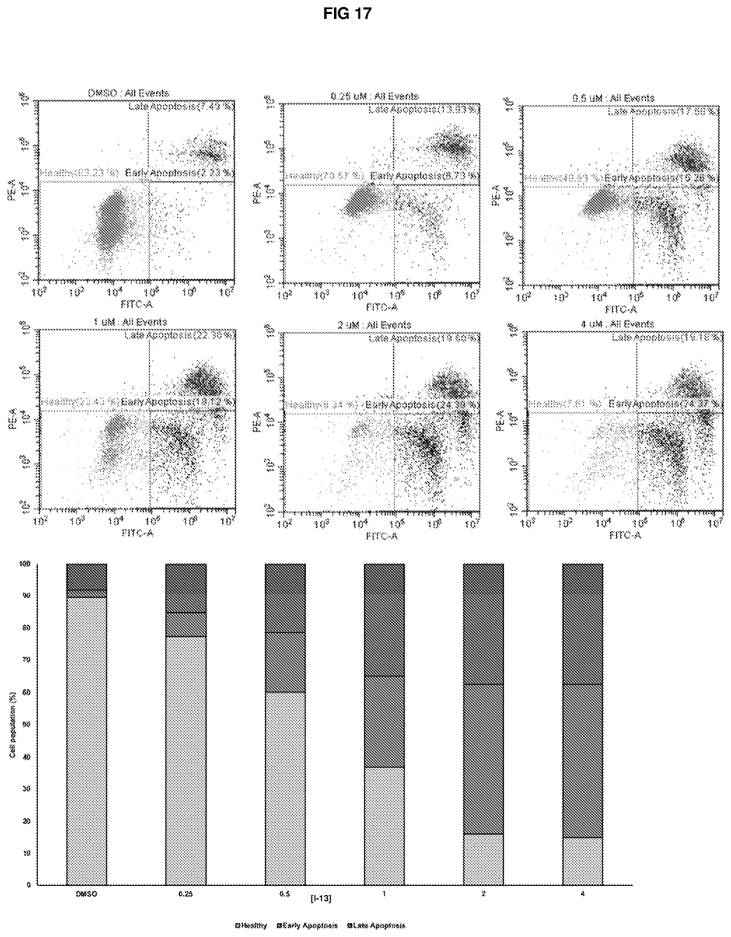

The apoptotic-inducting potential of I-13 and I-18 in MV-4-11 was assessed by FACs which is shown in FIG. 7 and FIG. 8 respectively. Both compounds showed larger populations of cells undergoing early apoptosis (lower right quadrant: FITC-Annexin V high and PI low) and late apoptosis (upper right quadrant: FITC-Annexin V high and PI low) with increasing concentration of inhibitor and decreasing populations of healthy cells (lower left quadrant: FITC-Annexin V low and PI low) with increasing concentration of inhibitor. Similar FACs analysis is shown for I-50, I-34 and I-13 in MV-4-11 cells in FIGS. 15 to 17 respectively.

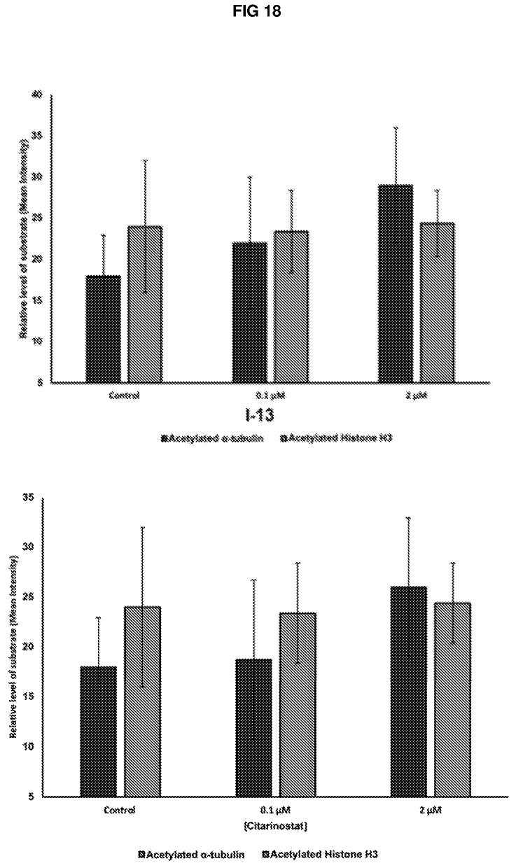

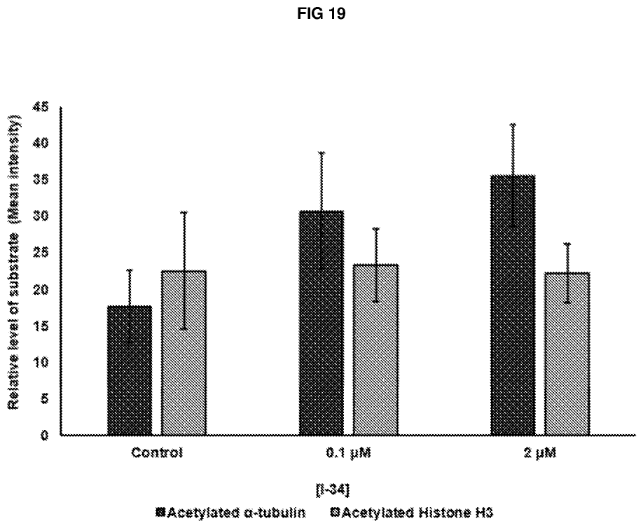

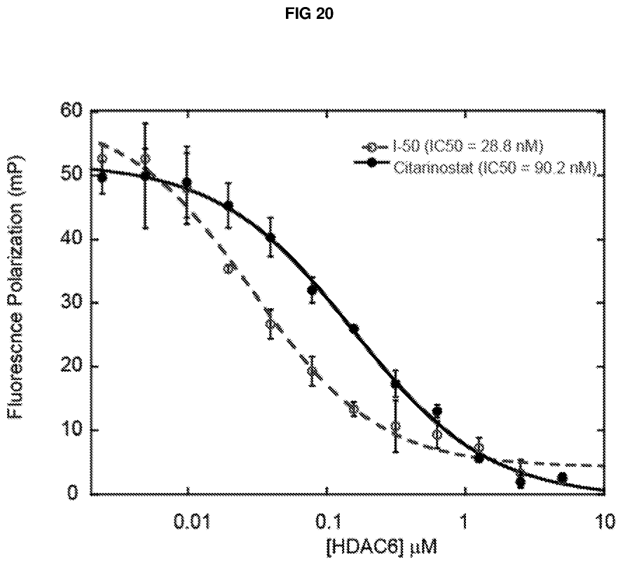

These in cellulo findings were also confirmed by immunofluorescence analysis where the exemplary compound I-13 exerted a substantially strong effect on tubulin acetylation than citarinostat which displayed no preferential acetylation on α-tubulin until high concentrations (2 μM) in FIG. 18. Similar analysis is shown for exemplary compound I-34 in FIG. 19.

H. Plasma Stability

The stability of select compounds of the application in plasma was assessed quantitatively through LC-MS and the results are shown in Table 6.

TABLE 6
Stability results of the compounds in mouse Plasma

| | | Remaining Percentage (%) | | | | | | Half-Life |
|---|---|---|---|---|---|---|---|---|
| Compound | Species | 0 min | 15 min | 30 min | 45 min | 60 min | 120 min | (min) |
| 1-18 | Mouse | 100 | 96.3 | 63.0 | 54.3 | 51.3 | 50.1 | 109 |
| 1-38 | Mouse | 100 | 89.3 | 85.0 | 84.9 | 83.3 | 66.0 | 210 |
| Belinostat | Mouse | 100 | 88.6 | 78.8 | 75.0 | 72.1 | 53.8 | 141 |
| Vorinostat | Mouse | 100 | 98.6 | 89.0 | 74.9 | 68.4 | 52.1 | 119 |
| (SAHA) | | | | | | | | |

I. Pharmacokinetic Studies

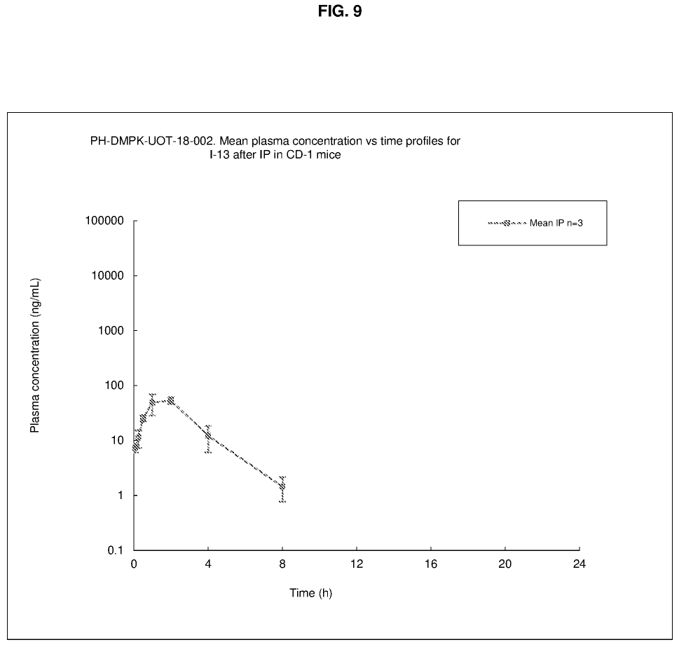

The pharmacokinetic stability of exemplary compound I-13 in male CD-1 mice (n=3) following intraperitoneal administration. The plasma concentration in mice treated with I-13 is shown in FIG. 9.

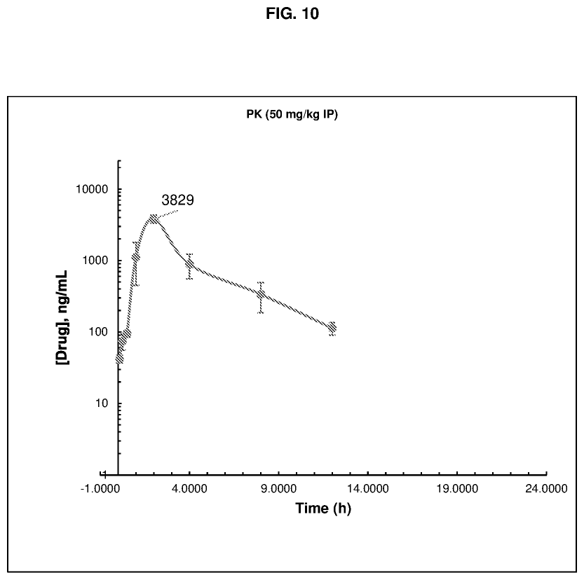

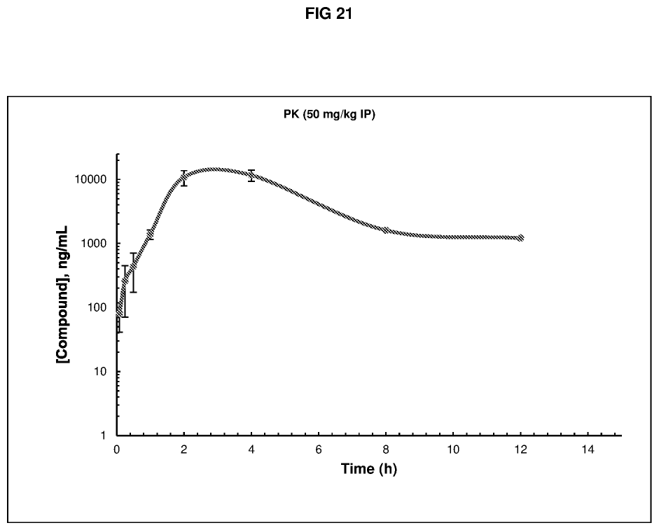

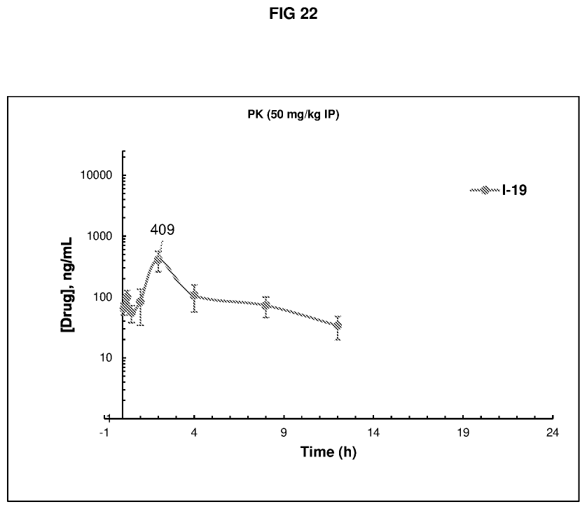

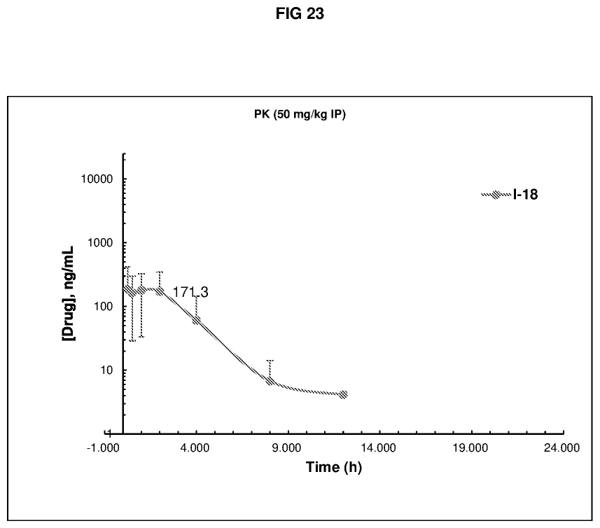

The pharmacokinetic stability of exemplary compound I-50 in male Balb/c nude mice (n=3) following intraperitoneal administration. The plasma concentration in mice treated with I-50 is shown in FIG. 10. Similar pharmacokinetic stability analysis is shown for exemplary compounds I-25, I-19 and I-18 FIGS. 21 to 23.

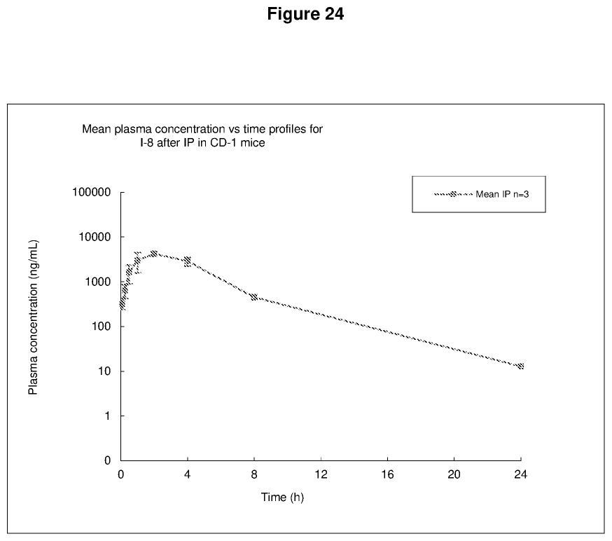

The pharmacokinetic stability of exemplary compound I-63 in male CD-1 mice (n=3) following intraperitoneal administration is shown in FIG. 24.

J. Kinome Screen

To assess the selectivity of the compounds for HDAC proteins, the exemplary compounds I-12 and I-18 were assessed in a kinase screen to determine the effect (if any) on a panel of 97 kinases. Both exemplary compounds demonstrated limited to no inhibitory activity all the kinases tested.

K. Fluorescence Polarization

Exemplary compound I-50 was also assessed for binding potency against HDAC6 through an in vitro fluorescence probe displacement assay (see for example, Shouksmith, A. E. et al. J. Med Chem. 2019, 62(5), 2651-2665). The IC₅₀ values for I-50 was used to calculate the binding potency to HDAC6 in comparison to Citarinostat. The IC50 curves are shown in FIG. 14.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A compound of Formula I or a pharmaceutically acceptable salt, solvate, and/or prodrug thereof: 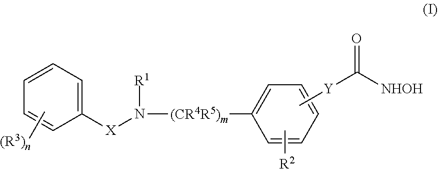
wherein
n is 4 or 5;
m is 0, 1, 2, 3, or 4;
X is selected from C(O) and SO₂;
R¹ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆ heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R² is selected from H, halo, C₁₋₄alkyl, and OC₁₋₄alkyl;
each R³ is the same or different and is selected from halo;
R⁴ and R⁵ are independently selected from H and C₁₋₄alkyl;
Y is absent or selected from C₁₋₆alkyene, C₂₋₆alkenylene and C₂₋₆alkynylene;
the —Y—C(O)NHOH group is bonded to a meta or para position of the phenyl ring;
all alkyl and alkylene are optionally fluoro substituted; and
all available hydrogen atoms are optionally replaced with deuterium,
provided when m is 0, and Y is absent, then R¹ is not H or C₁₋₁₀alkyl.

2. The compound of claim 1, wherein R¹ is selected from H, C₁₋₅alkyl, C₁₋₃alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo.

3. The compound of claim 1 or claim 2, wherein R¹ is selected from H, C₁₋₃alkyl, C₃₋₆ cycloalkyl, C₁₋₂alkyleneheteroaryl, and C₁₋₂alkylenearyl, the latter 4 groups optionally substituted with one or more groups independently selected from halo, C₁₋₄alkyl, N(C₁₋₄alkyl)(C₁₋₄alkyl), and OC₁₋₄alkyl.

4. The compound of any one of claims 1 to 3, wherein R¹ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups optionally substituted with one or more groups independently selected from F, C₁₋₄alkyl, N(CH₃)₂, and OCH₃.

5. The compound of claim 4, wherein R¹ is pyridinylmethyl, pyridazinylmethyl, pyrimidinylemethyl or pyrazinylmethyl.

6. The compound of claim 4, wherein R¹ is C₃₋₆cycloalkyl.

7. The compound of claim 4, wherein R¹ is benzyl.

8. The compound of claim 4, wherein R¹ is pyridinylmethyl.

9. The compound of claim 4, wherein R¹ is isopropyl or cyclopentyl.

10. The compound of any one of claims 1 to 9, wherein R² is selected from H, halo and OC₁₋₃alkyl.

11. The compound of claim 10, wherein R² is selected from H, fluorine and OCH₃.

12. The compound of any one of claims 1 to 11, wherein X is SO₂.

13. The compound of any one of claims 1 to 12, wherein each R³ is selected from F and Cl. I

14. The compound of any one of claims 1 to 13, wherein each R³ is F.

15. The compound of any one of claims 1 to 14, wherein R⁴ and R⁵ are independently selected from H and CH₃.

16. The compound of any one of claims 1 to 15, wherein m is 0 or 1.

17. The compound of any one of claims 1 to 16, wherein Y is absent.

18. The compound of any one of claims 1 to 16, wherein Y is selected from —CH₂—, —CH₂CH₂— and —CH═CH—.

19. The compound of any one of claims 1 to 18, wherein the —Y—C(O)NHOH group is bonded to the para position of the phenyl ring.

20. The compound of claim 1, wherein the compound of Formula I is a compound of Formula I-A or a pharmaceutically acceptable salt, solvate, and/or prodrug thereof: 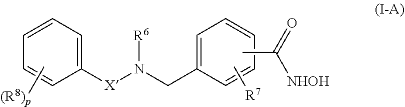
wherein
p is 4 or 5;
X′ is selected from C(O) and SO₂;
R⁶ is selected from H, C₁₋₁₀alkyl, C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, the latter 6 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆ heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R⁷ is selected from H, halo, C₁₋₄alkyl, and OC₁₋₄alkyl;
each R⁸ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

21. The compound of claim 20, wherein R⁶ is selected from H, C₁₋₅alkyl, C₁₋₃alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo.

22. The compound of claim 20 or claim 21, wherein R⁶ is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, C₁₋₄alkyl, N(C₁₋₄alkyl)(C₁₋₄alkyl), and OC₁₋₄alkyl.

23. The compound of any one of claims 20 to 22, wherein R⁶ is selected from H, methyl, ethyl, isopropyl, C₃₋₆cycloalkyl, benzyl, pyridinylmethyl, pyridazinylmethyl, pyrimidinylemethyl and pyrazinylmethyl, the latter 9 groups optionally substituted with one or more groups independently selected from F, C₁₋₄alkyl, N(CH₃)₂, and OCH₃.

24. The compound of claim 23, wherein R⁶ is pyridinylmethyl, pyridazinylmethyl, pyrimidinylemethyl or pyrazinylmethyl.

25. The compound of claim 23, wherein R⁶ is C₃₋₆cycloalkyl.

26. The compound of claim 23, wherein R⁶ is benzyl.

27. The compound of claim 23, wherein R⁶ is pyridinylmethyl.

28. The compound of claim 23, wherein R⁶ is isopropyl or cyclopentyl.

29. The compound of any one of claims 21 to 28, wherein R⁷ is selected from H, halo and OC₁₋₃alkyl.

30. The compound of any one of claims 21 to 29, wherein R⁷ is selected from H, fluorine and OCH₃.

31. The compound of any one of claims 21 to 30, wherein X′ is SO₂.

32. The compound of any one of claims 21 to 30, wherein X′ is C(O).

33. The compound of any one of claims 21 to 32, wherein each R⁸ is selected from F and Cl.

34. The compound of any one of claims 21 to 32, wherein each R⁸ is F.

35. The compound of any one of claims 21 to 32, wherein one R⁸ is Cl and the remaining R⁸ are F.

36. The compound of any one of claims 21 to 32, wherein two R⁸ are Cl and the remaining R³ are F.

37. The compound of any one of claims 21 to 36, wherein the C(O)NHOH group is bonded to the para position of the phenyl ring.

38. The compound of claim 20 selected from: 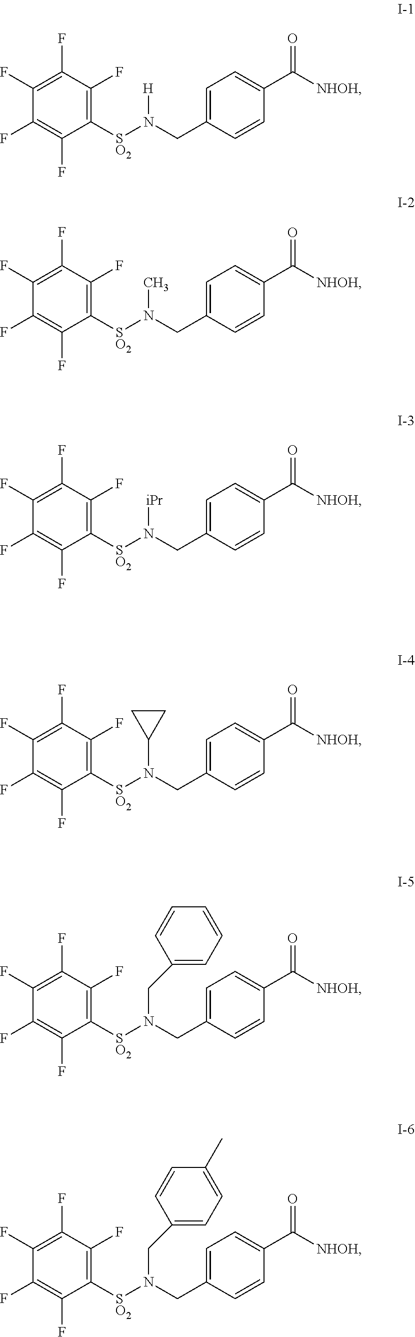 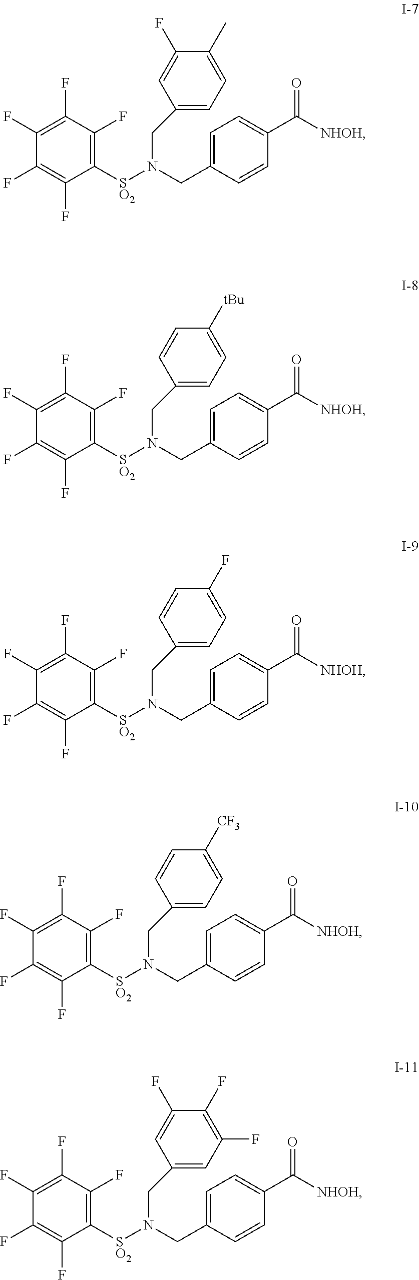 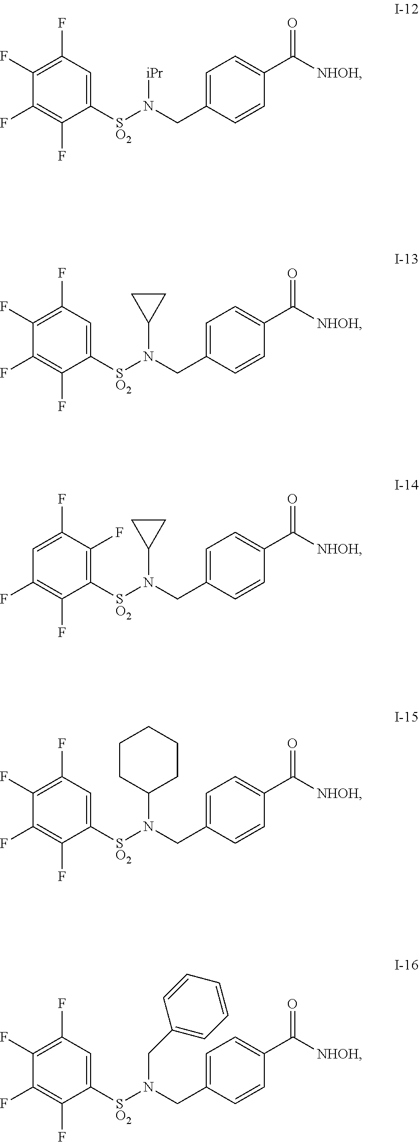 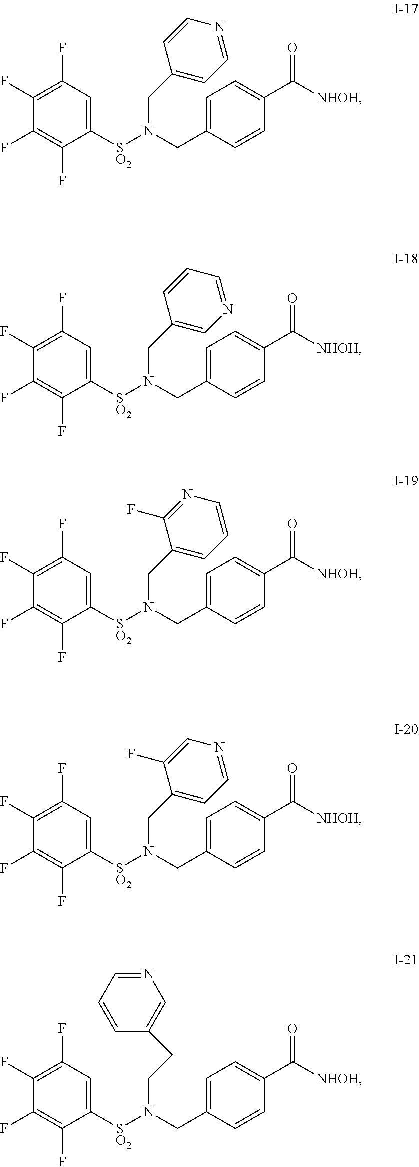 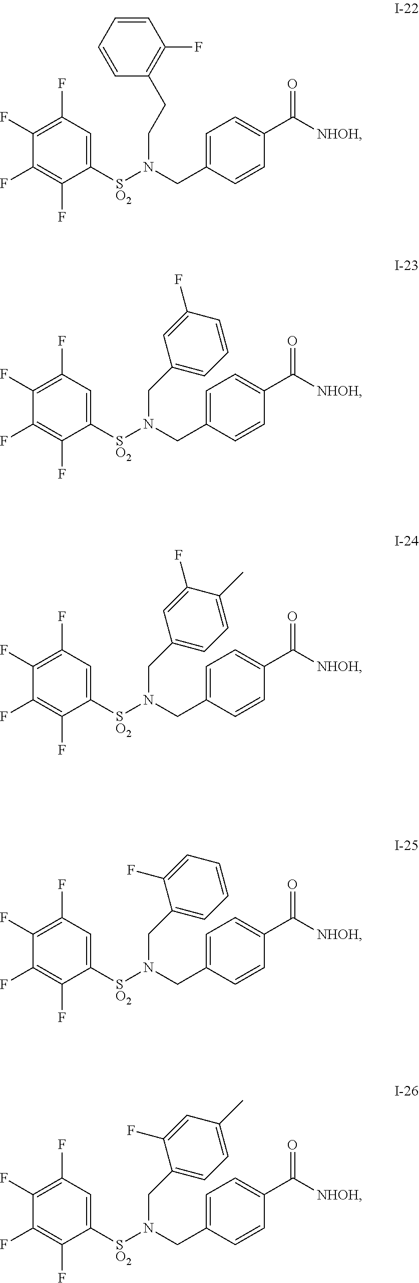 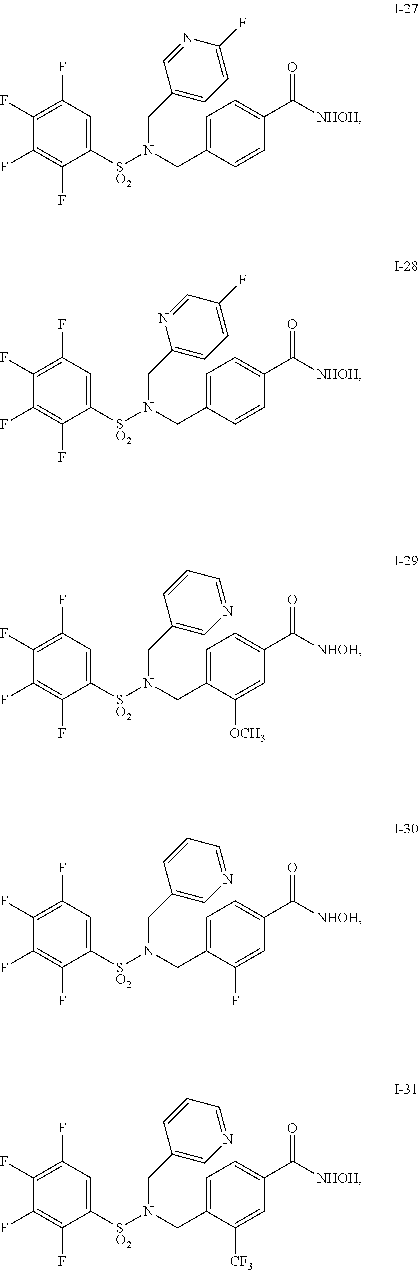 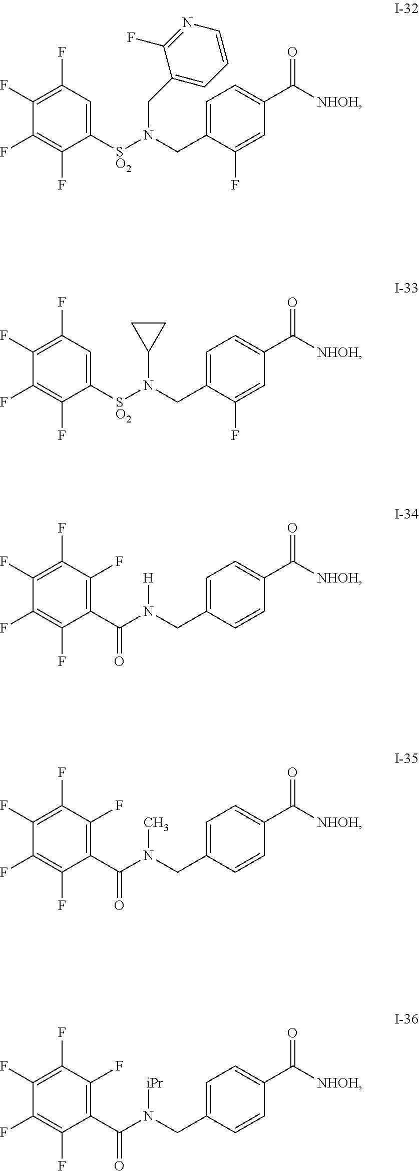 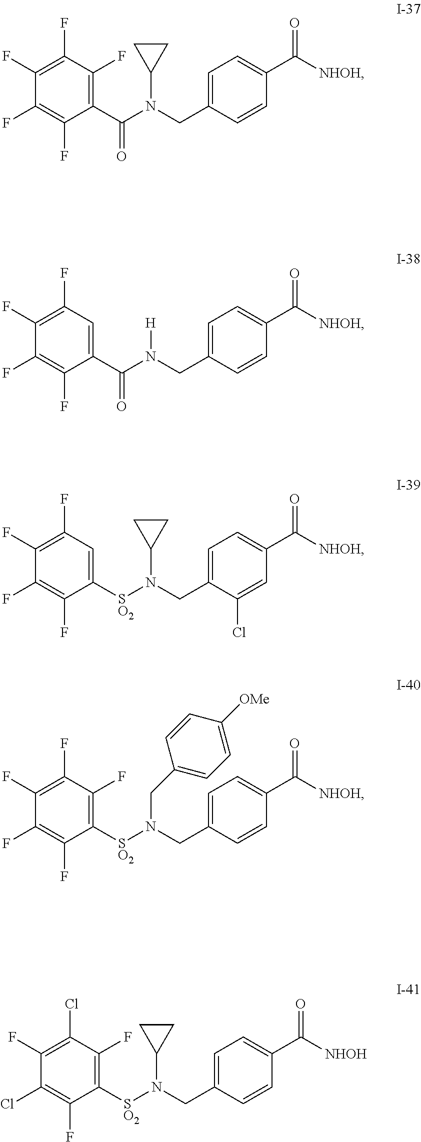 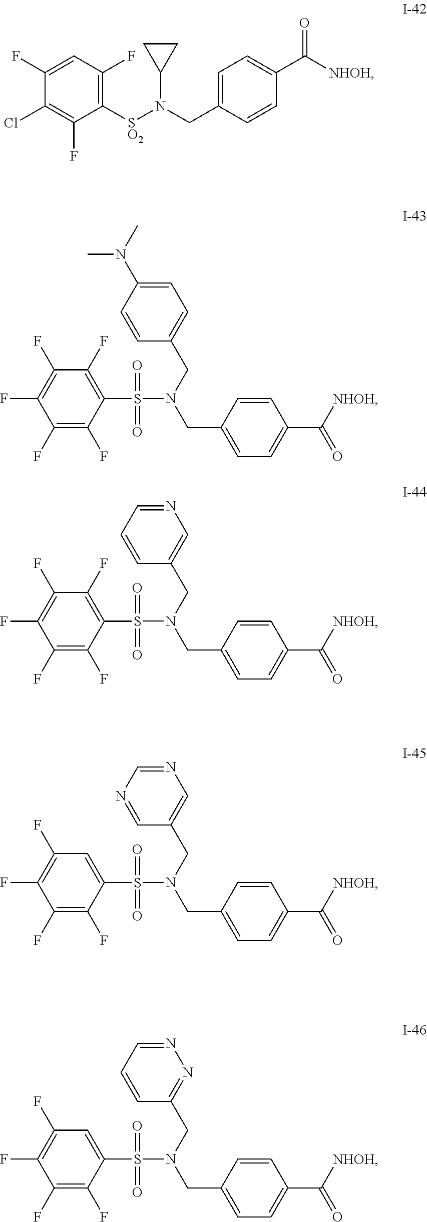 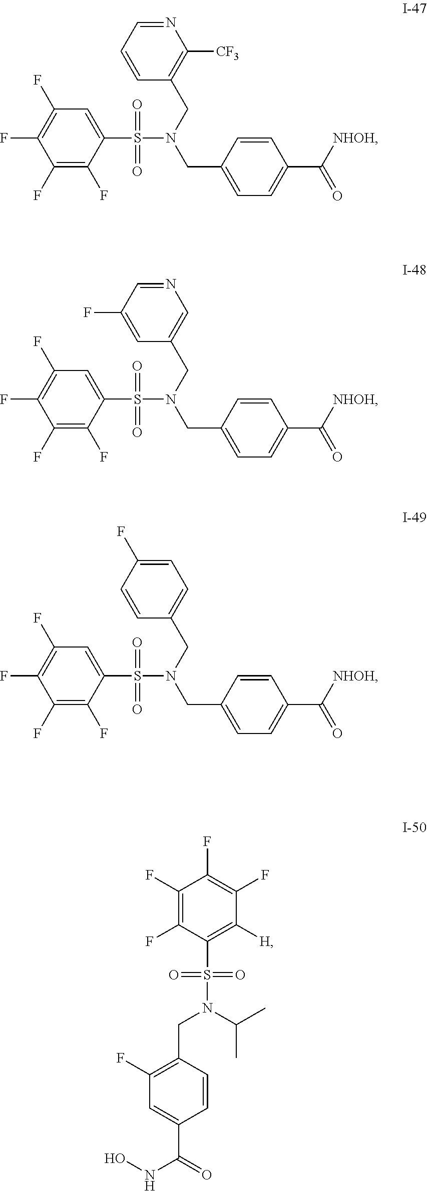 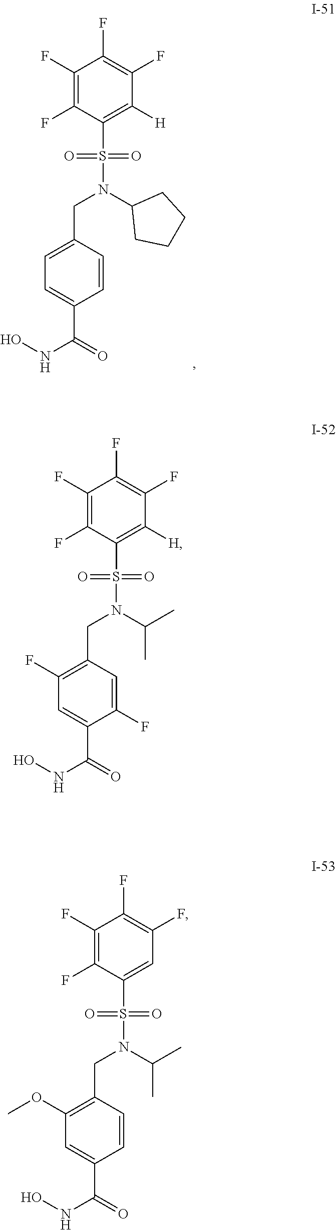 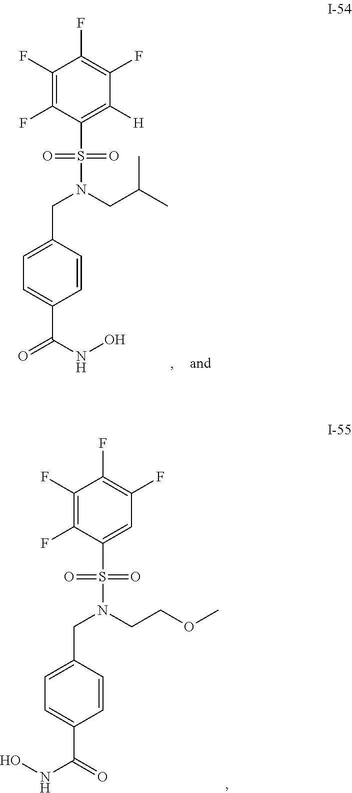 [SMILES: CC(C)CN(Cc1ccc(C(=O)NO)cc1)S(=O)(=O)c1cc(F)c(F)c(F)c1F ; COCCN(Cc1ccc(C(=O)NO)cc1)S(=O)(=O)c1cc(F)c(F)c(F)c1F]
or a pharmaceutically acceptable salt and/or solvate thereof.

39. The compound of claim 20 selected from: 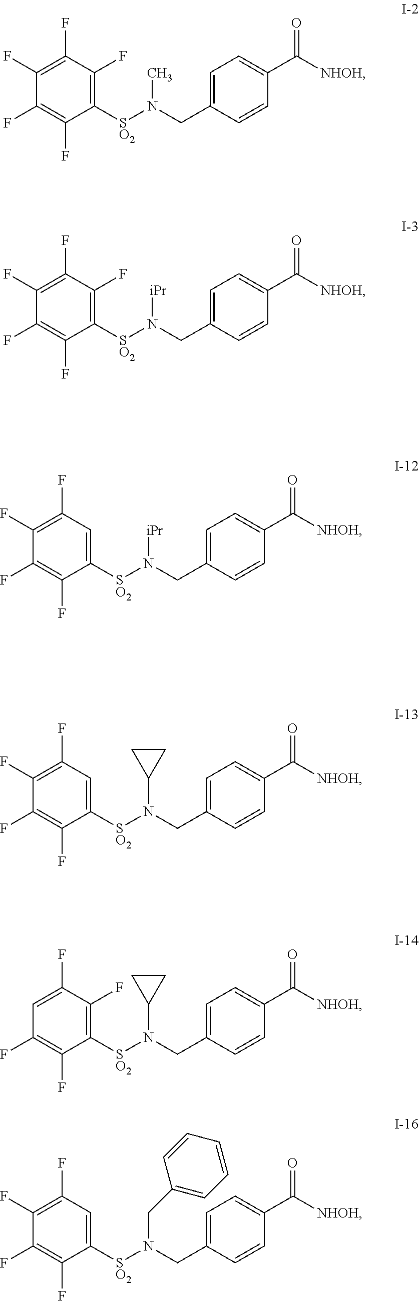 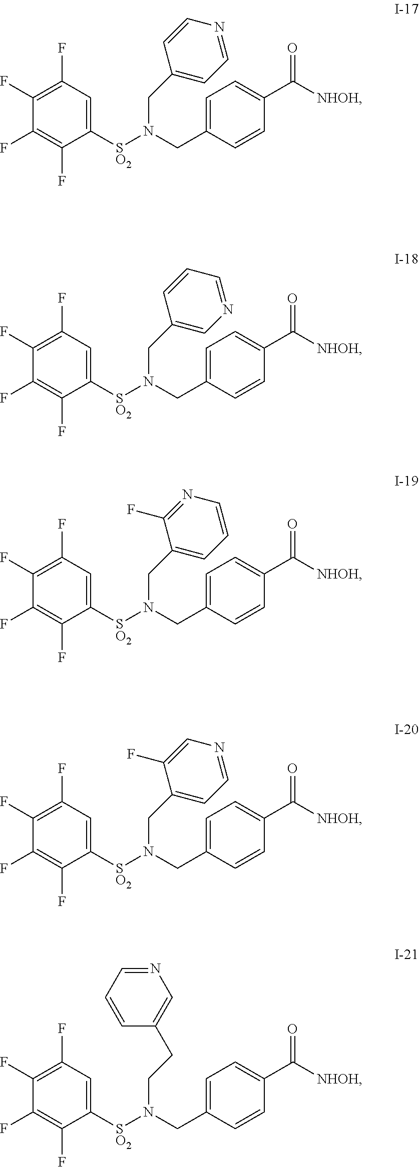 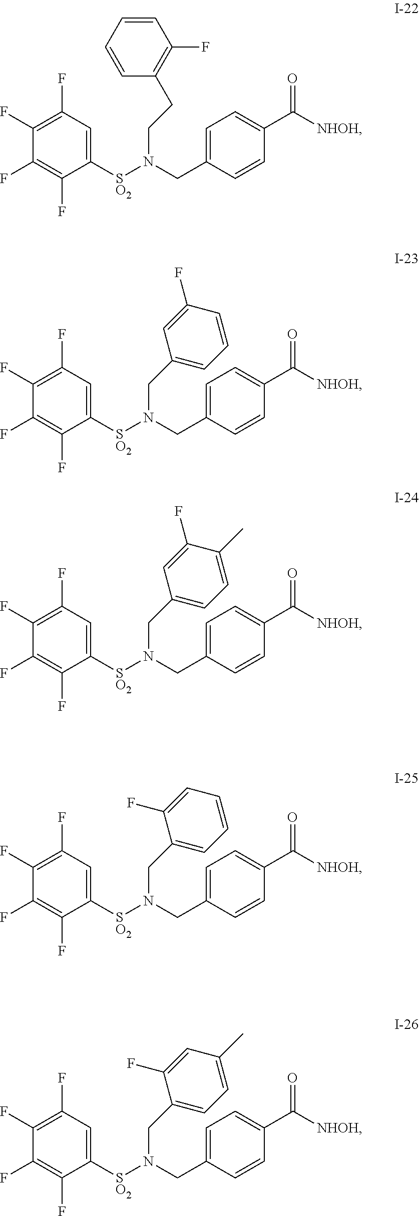 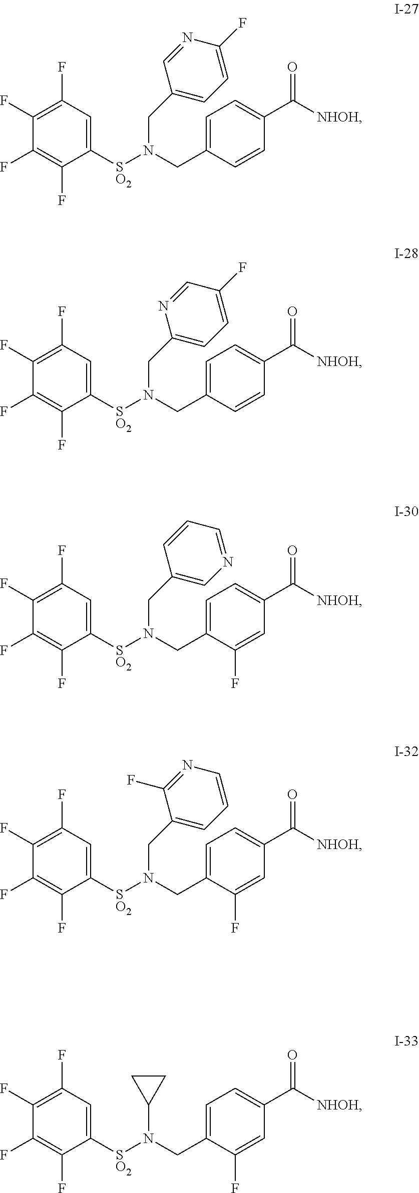 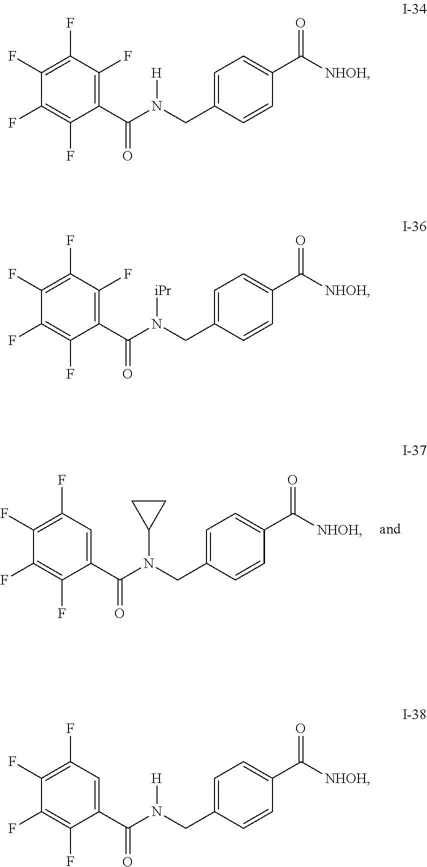
or a pharmaceutically acceptable salt and/or solvate thereof.

40. The compound of claim 1, wherein the compound of Formula I is a compound of Formula I-B, or a pharmaceutically acceptable salt and/or solvate thereof: 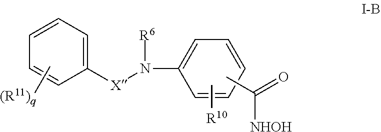
wherein
q is 4 or 5;
X″ is selected from C(O) and SO₂;
R⁹ is selected from C₃₋₁₀cycloalkyl, C₁₋₆alkyleneC₃₋₁₀cycloalkyl, C₁₋₆alkyleneheteroaryl, C₁₋₆alkylenearyl, and C₁₋₆alkyleneheterocycloalkyl, each of which is optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R¹⁰ is selected from H, halo, C₁₋₄alkyl, and OC₁₋₄alkyl;
each R¹¹ is the same or different and is selected from halo;
the C(O)NHOH group is bonded to a meta or para position of the phenyl ring;
all alkyl and alkylene are optionally fluoro substituted; and
all available hydrogen atoms are optionally replaced with deuterium.

41. The compound of claim 1, wherein the compound of Formula I is a compound of Formula I-C or a pharmaceutically acceptable salt, solvate, and/or prodrug thereof: 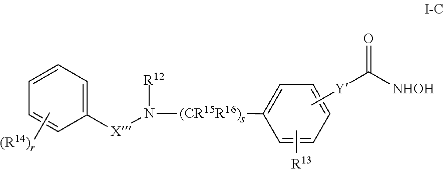
wherein
r is 4 or 5;
s is 0, 1, 2, 3, or 4;
X′″ is selected from C(O) and SO₂;
R¹² is selected from H, C₁₋₁₀alkyl, C₃₋₁₀cycloalkyl, C₁₋₆alkyleneC₃₋₁₀cycloalkyl, C₁₋₆ alkyleneheteroaryl, C₁₋₆alkylenearyl, and C₁₋₆alkyleneheterocycloalkyl, the latter 6 groups being optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, and C₅₋₆ heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo;
R¹³ is selected from H, halo, C₁₋₄alkyl, and OC₁₋₄alkyl;
each R¹⁴ is the same or different and is selected from halo;
R¹⁵ and R¹⁶ are independently selected from H and C₁₋₄alkyl;
Y′ is selected from C₁₋₆alkyene, C₂₋₆alkenylene and C₂₋₆alkynylene;
the —Y′—C(O)NHOH group is bonded to a meta or para position of the phenyl ring;
all alkyl and alkylene are optionally fluoro substituted; and
all available hydrogen atoms are optionally replaced with deuterium.

42. The compound of claim 41, wherein R¹² is selected from H, C₁₋₅alkyl, C₁₋₃ alkyleneheteroaryl, C₃₋₆cycloalkyl and C₁₋₃alkylenearyl, the latter 4 groups optionally substituted with one or more groups independently selected from halo, C₁₋₄alkyl, N(C₁₋₄alkyl)(C₁₋₄alkyl), OC₁₋₄alkyl, C₃₋₆cycloalkyl, C₃₋₆heterocycloalkyl, phenyl, C₅₋₆heteroayl, in which groups C₃₋₆cycloalkyl, C₃₋₆heterocycloalkyl, phenyl, and C₅₋₆heteroayl are each unsubstituted or substituted with one or more C₁₋₄alkyl or halo.

43. The compound of claim 41, wherein R¹² is selected from H, C₁₋₃alkyl, C₃₋₆cycloalkyl, C₁₋₂alkyleneheteroaryl, and C₁₋₂alkylenearyl, the latter 4 groups optionally substituted with one or more groups independently selected from halo, and C₁₋₃alkyl.

44. The compound of claim 41, wherein R¹² is selected from H, methyl, isopropyl, C₃₋₆ cycloalkyl, benzyl, and pyridinylmethyl, the latter 2 groups are optionally substituted with one or more groups selected from fluorine and C₁₋₂alkyl.

45. The compound of any one of claims 41 to 44, wherein R¹³ is selected from H, and halo.

46. The compound of any one of claims 41 to 44, wherein R¹³ is selected from H and fluorine.

47. The compound of any one of claims 41 to 46, wherein X′″ is SO₂.

48. The compound of any one of claims 41 to 46, wherein X′″ is C(O).

49. The compound of any one of claims 41 to 48, wherein each R¹⁴ is selected from F and Cl.

50. The compound of any one of claims 41 to 48, wherein each R¹⁴ is F.

51. The compound of any one of claims 41 to 48, wherein one R¹⁴ is Cl and the remaining R³ are F.

52. The compound of any one of claims 41 to 48, wherein two R¹⁴ are Cl and the remaining R³ are F.

53. The compound of any one of claims 41 to 52, wherein R¹⁵ and R¹⁶ are independently selected from H and CH₃.

54. The compound of claim 53, wherein both R¹⁴ and R¹⁵ are H.

55. The compound of any one of claims 41 to 54, wherein s is 0, 1 or 2.

56. The compounds of claim 55, wherein s is 0 or 1.

57. The compound of claim 55, wherein s is 1.

58. The compound of any one of claims 41 to 57, wherein Y is selected from C₁₋₄alkyene, C₂₋₄alkenylene and C₂₋₄alkynylene.

59. The compound of claim 58, wherein Y is selected from C₁₋₄alkyene and C₂₋₄alkenylene.

60. The compound of claim 58, wherein Y is selected from —CH₂—, —CH₂CH₂— and —CH═CH—.

61. The compound of claim 60, wherein Y is —CH═CH—.

62. The compound of claim 61, wherein the double bond is in the trans configuration.

63. The compound of any one of claims 41 to 62, wherein the —Y—C(O)NHOH group is bonded to the para position of the phenyl ring.

64. The compound of claim 41 selected from 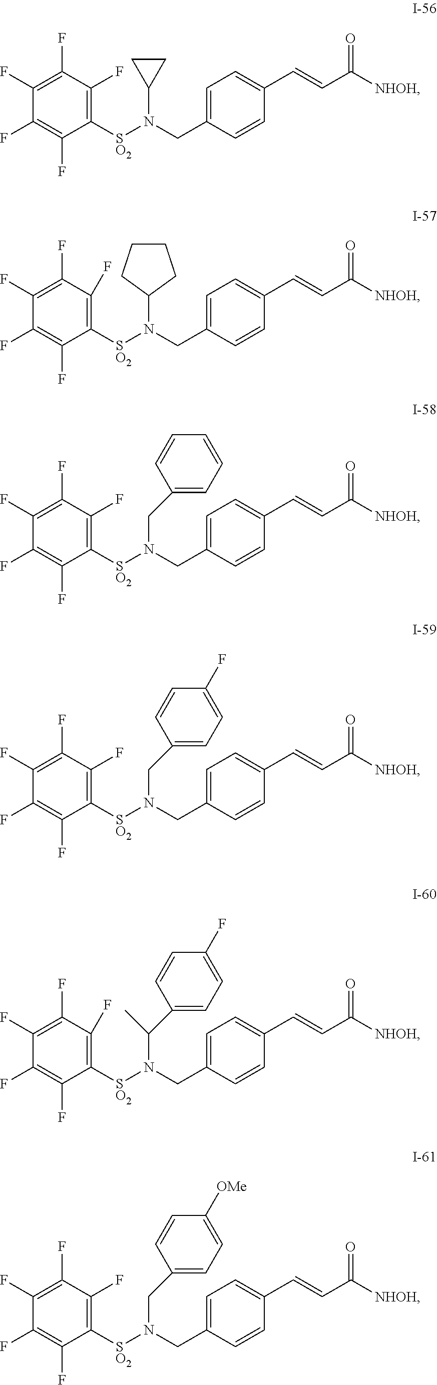 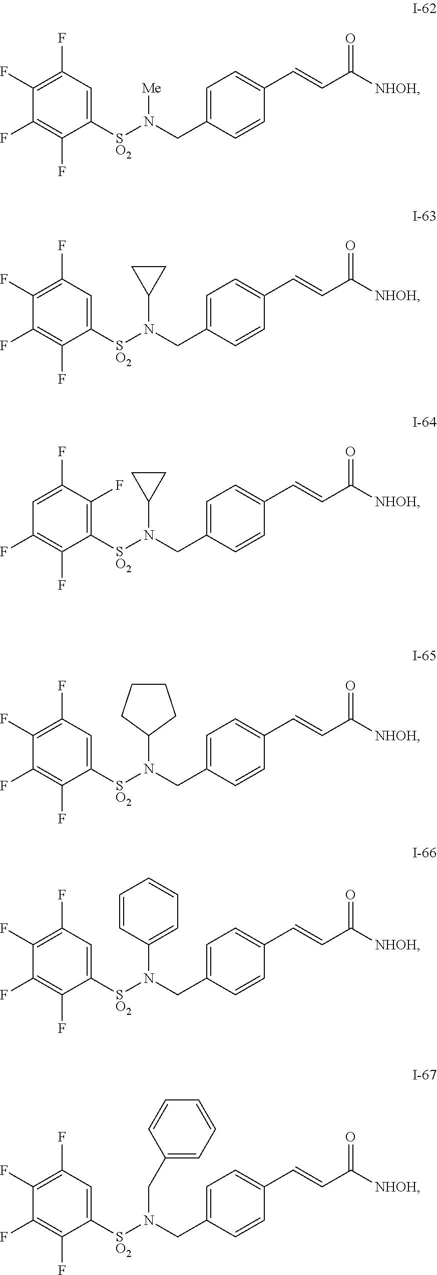 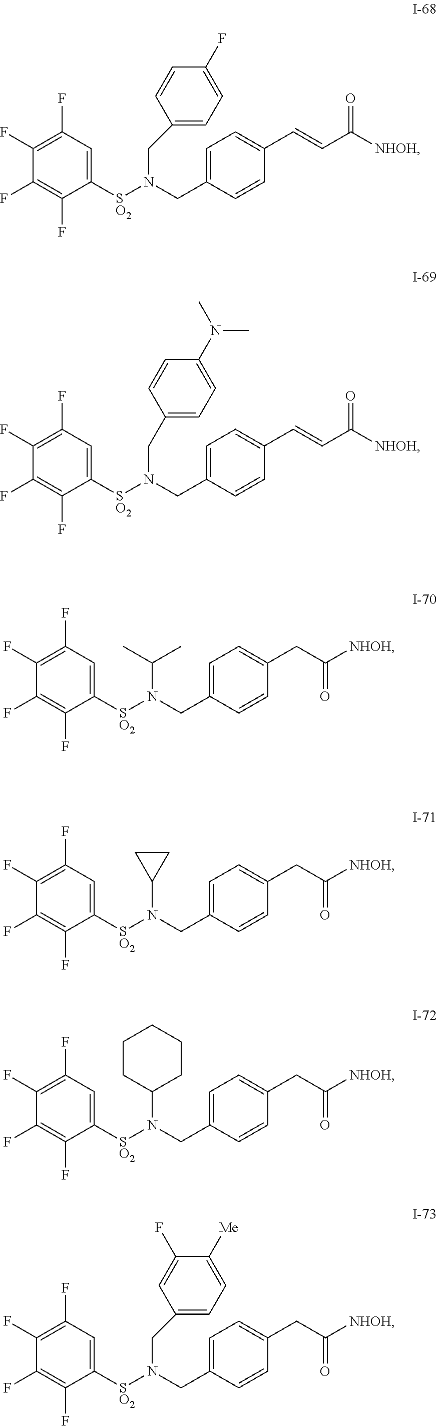 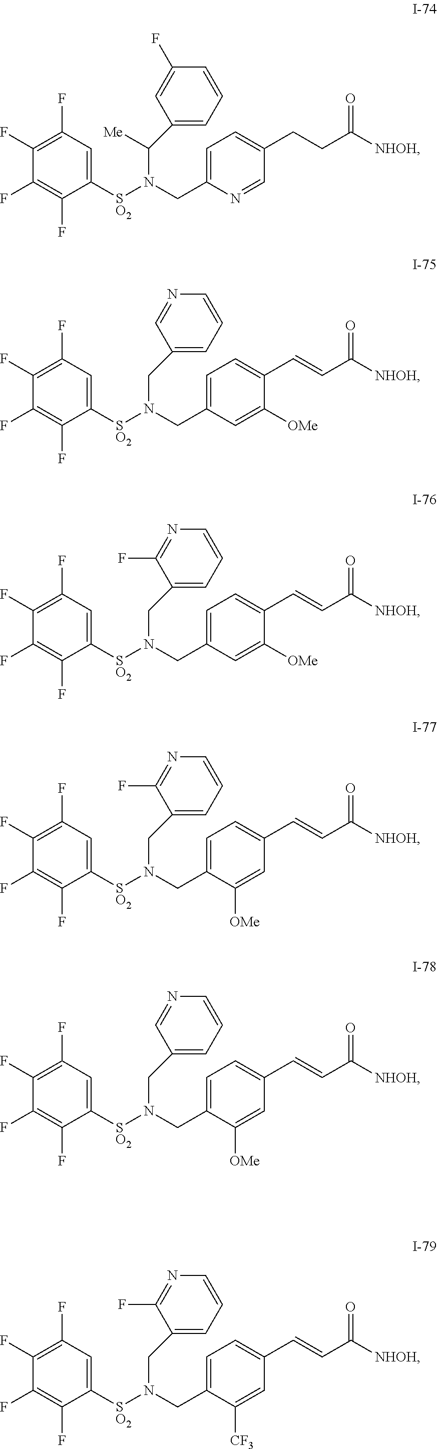 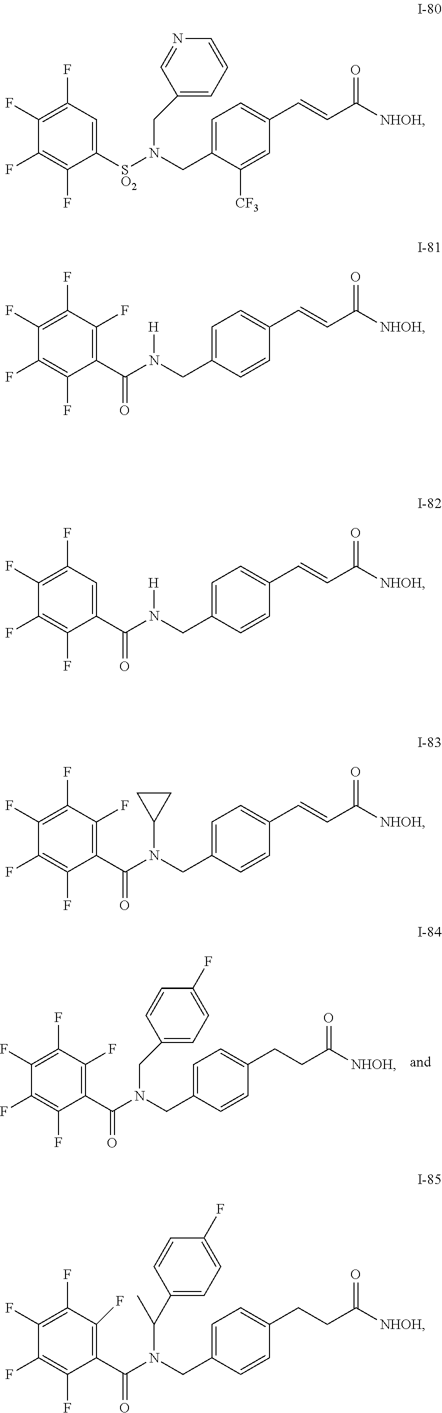
or a pharmaceutically acceptable salt, solvate and/or prodrug thereof.

65. The compound of claim 41, selected from 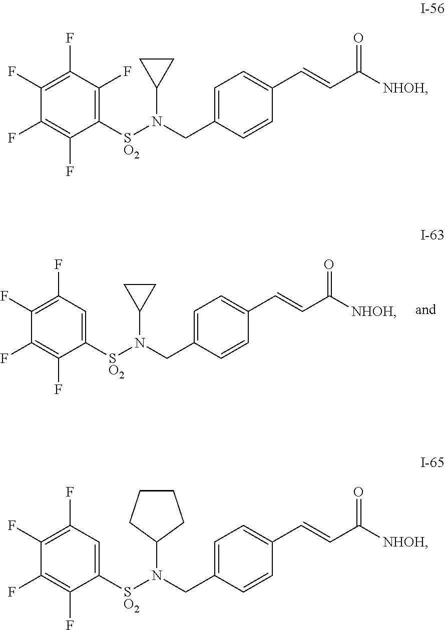
or a pharmaceutically acceptable salt, solvate, and/or prodrug thereof.

66. A pharmaceutical composition comprising one or more compounds of any one of claims 1 to 65, and/or a pharmaceutically acceptable salt, solvate and/or prodrug thereof, and a pharmaceutically acceptable carrier.

67. A method of treating a disease, disorder or condition that benefits from inhibiting HDAC6 comprising administering an effective amount of one or more compounds of any one of claims 1 to 65, and/or a pharmaceutically acceptable salt, solvate and/or prodrug thereof, or one or more compositions of claim 11, to a subject in need thereof.

68. The method of claim 67, wherein the disease, disorder or condition is cancer.

69. The method of claim 68, wherein the cancer is hematological cancer or brain cancer. In some embodiments, the cancer is leukemia (such as acute myeloid leukemia, acute lymphoblastic leukemia (ALL), or chronic myeloid leukemia (CML)), adenosarcoma, bile duct, fibroblast, kidney, mesothelioma, multiple myeloma, liver, central nervous system, soft tissue, pancreas, thyroid, gastric, ovary, upper aerodigestive tract, urinary tract, lung, skin, colorectal, esophagus, breast, uterus, cervix, bone, peripheral nervous system or lymphoma.

70. The method of claim 68, wherein the cancer is breast cancer, multiple myeloma, pancreatic cancer, lung cancer, prostate cancer, renal cancer, ovarian cancer and leukemias such as acute myeloid leukemia (AML) and acute lymphoblastic leukemia (ALL).

71. The method of claim 67, wherein the disease, disorder or condition that benefits from selectively inhibiting HDAC6 is selected from a cardiovascular disease, a bacterial infection, a neurological disease, inflammation and immunological disorders such as rheumatoid arthritis, psoriasis, multiple sclerosis, lupus and organ transplant rejection.

72. The method of any one of claims 67 to 71, wherein the one or more compounds or one or more compositions are administered in combination with other active agents selected from one or more of chemotherapeutics, microtubule destabilizing agents, Hsp90 inhibitors, inhibitors of Hsp90 downstream proteins, tyrosine kinase inhibitors, HER-2 inhibitors, BCR-ABL inhibitors, Akt inhibitors, c-Raf and MEK inhibitors, Aurora A and B inhibitors, EGFR inhibitors, proteasome inhibitors, ubiquitin proteasome system inhibitors, modulators of autophagy and protein homeostasis agents.
